(19) United States
(12) Patent Application Publication
(10) Pub. No.: US 2018/0354934 A1
(43) Pub. Date: Dec. 13, 2018

(54) TERBENZOCYCLOPENTADIENE COMPOUND, HIGH POLYMER, MIXTURE, COMPOSITION AND ORGANIC ELECTRONIC DEVICE

(71) Applicant: GUANGZHOU CHINARAY OPTOELECTRONIC MATERIALS LTD., Guangzhou (CN)

(72) Inventors: Ruifeng HE, Guangzhou (CN); Peng SHU, Guangzhou (CN); Jun WANG, Guangzhou (CN); Junyou PAN, Guangzhou (CN)

(21) Appl. No.: 15/780,521

(22) PCT Filed: Nov. 25, 2016
(86) PCT No.: PCT/CN2016/107304

§ 371 Date: May 31, 2018

(30) Foreign Application Priority Data
Dec. 4, 2015 (CN) 201510888896.6

(51) Int. Cl. C07D 403/14; C07D 401/14; C07D 407/14; C07D 409/14; C07D 519/00; H01L 51/00; H01L 51/50

(52) U.S. Cl. CPC C07D 403/14; C07D 401/14; C07D 407/14; C07D 409/14; C07D 519/00; H01L 51/0038; H01L 51/5072; H01L 51/006; H01L 51/0067; H01L 51/0072; H01L 51/5012; H01L 51/5056; H01L 51/0058

(57) ABSTRACT

Disclosed are a terbenzocyclopentadiene compound of better solubility and film-forming property, and a high polymer, mixture, composition and organic electronic device comprising same. This terbenzocyclopentadiene compound contains a benzocyclopentadiene structure. The matching of energy level and the symmetry of the structure provide a possibility for increasing the chemical/environmental stability of terbenzocyclopentadiene compounds and photoelectric devices. This terbenzocyclopentadiene compound has a better solubility in an organic solvent, and also facilitates forming a high-quality film by a printing method due to a high molecular weight. After this terbenzocyclopentadiene compound is used in OLED, particularly as a luminescent layer material, a higher quantum efficiency, luminescence stability and device life time can be provided.

### TECHNICAL FIELD

The present disclosure relates to the field of organic photoelectric material, particularly to a terbenzocyclopentadiene compound, and a polymer, a mixture, a formulation and an organic electronic device comprising the same.

### BACKGROUND

Organic semiconductor materials have the characteristics of structural diversity, relatively low manufacturing cost, excellent photoelectric property, and the like, and have great potential for application in photoelectric devices e.g. organic light-emitting diodes (OLEDs), such as flat panel displays and lighting.

In order to improve the luminous performance of organic light-emitting diodes and promote the large-scale industrialization process of organic light-emitting diodes, organic photoelectric material systems having various new structures have been widely developed. Among them, benzocyclopentadiene structural compounds, such as fluorene, spirofluorene, indenofuorene and the like, have been widely used in optoelectronic devices due to their excellent photoelectric response and carrier transmission performance. However, the currently reported benzocyclopentadiene structural compounds has certain limitation in the stability. A new-type of benzocyclopentadiene structure should be developed for further exploring the photoelectric property of such material.

In addition, in order to reduce the production costs and realize a large-scale OLED device, printed OLED is becoming one of the most promising technical options. In this regard, materials are the key for printing OLED. However, the current small-molecule OLED materials developed based on evaporation technology have relatively poor solubility and film-forming property due to their lower molecular weight and rigid aromatic molecular structure, and particularly, it is difficult to form a non-cavity amorphous film with a regular morphology. Therefore, currently, it is lack of corresponding solution to the printed OLED material, and small molecule organic light-emitting diodes of high performance are still prepared by evaporation in vacuo. Therefore, it's especially important to design and synthesize organic small-molecule functional compounds having good solubility and film-forming property for realizing high-performance solution-processed organic light-emitting diodes.

### SUMMARY

Based on this, it is necessary to provide a terbenzocyclopentadiene compound having good solubility and film-forming property, and a polymer, a mixture, a formulation and an organic electronic device comprising the same.

A terbenzocyclopentadiene compound has the following general formula (1):

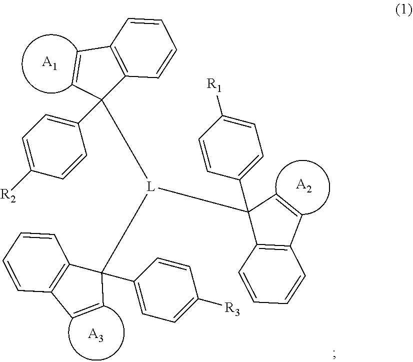

wherein L is a linking unit, and selected from an aryl group containing 6 to 40 carbon atoms or a heteroaryl group containing 3 to 40 carbon atoms;

A₁, A₂, or A₃ is selected from an aryl group containing 6 to 30 carbon atoms or a heteroaryl group containing 3 to 30 carbon atoms;

R₁, R₂, or R₃ is selected from H, D, F, CN, an alkyl group containing 1 to 30 carbon atoms, a cycloalkyl group containing 3 to 30 carbon atoms, an aromatic hydrocarbon group containing 6 to 60 carbon atoms, and an aromatic heterocyclic group containing 3 to 60 atoms, and one or more positions of R₁, R₂, or R₃ may be substituted by H, D, F, CN, alkyl, aralkyl, alkenyl, alkynyl, nitrile group, amino, nitro, acyl, alkoxy, carbonyl, sulfonyl group, cycloalkyl, or hydroxy.

A polymer comprises at least one repeating unit represented by general formula (1) included in the terbenzocyclopentadiene compound described above.

A mixture comprises the above terbenzocyclopentadiene compound or the above polymer:

The mixture further includes an organic functional material.

A formulation comprises the above terbenzocyclopentadiene compound, the above polymer or the above mixture:

The mixture further comprises an organic solvent.

An organic electronic device comprises the above terbenzocyclopentadiene compound or the above polymer.

Such terbenzocyclopentadiene compound comprises a benzocyclopentadiene structure. The matching of energy level and the symmetry of the structure provide the possibility for increasing the chemical/environmental stability of the terbenzocyclopentadiene compound and the photoelectric device. Such a terbenzocyclopentadiene compound has a better solubility in an organic solvent; meanwhile it has a high molecular weight and facilitates to form a high-quality film by a printing method. The terbenzocyclopentadiene compound, when is applied in the OLED, particularly as a luminous layer material, may provide a higher quantum efficiency, luminous stability and life time of devices.

### DETAILED DESCRIPTION OF EMBODIMENTS

In order to make the above objects, features, and advantages of the present disclosure to be understood more clearly, the specific embodiments of the present disclosure will be described in detail below with reference to the accompanying drawings and specific examples. In the following description, numerous specific details are set forth in order to provide a thorough understanding of the present disclosure. Rather, the present disclosure can be implemented in many other different ways from those described herein, and similar improvements may be made by those skilled in the art without departing from the spirit of the present disclosure. Therefore, the present disclosure is not limited by the specific embodiments disclosed below.

In the present disclosure, the formulation and the printing ink, or the ink, have the same meaning and they are interchangeable.

In the present disclosure, the host material or the matrix material have the same meaning and they are interchangeable.

In the present disclosure, the metal organic clathrate, the metal organic complexes, and organometallic complexes have the same meaning and are interchangeable.

In the present disclosure, the polymer, the high polymer, and the polymer material have the same meaning and they are interchangeable.

The disclosure discloses a terbenzocyclopcntadiene compound having the following general formula (1):

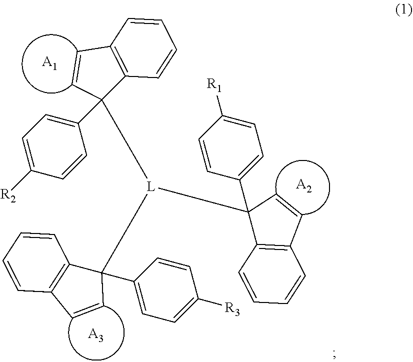

wherein

L is a linking unit, and selected from an aryl group containing 6 to 40 carbon atoms or a heteroaryl group containing 3 to 40 carbon atoms;

A₁, A₂, or A₃ is selected from an aryl group containing 6 to 30 carbon atoms or a heteroaryl group containing 3 to 30 carbon atoms;

R₁, R₂, or R₃ is selected from H, D, F, CN, an alkyl group containing 1 to 30 carbon atoms, a cycloalkyl group containing 3 to 30 carbon atoms, an aromatic hydrocarbon group containing 6 to 60 carbon atoms, and an aromatic heterocyclic group containing 3 to 60 atoms, and one or more positions of R₁, R₂, or R₃ may be substituted by H, D, F, CN, alkyl, aralkyl, alkenyl, alkynyl, nitrile group, amino, nitro, acyl, alkoxy, carbonyl, sulfonyl group, cycloalkyl, or hydroxy.

Preferably. L is an aryl group containing 6 to 30 carbon atoms or a heteroaryl group having 3 to 30 carbon atoms.

More preferably, L is an aryl group containing 6 to 25 carbon atoms or a heteroaryl group having 3 to 25 carbon atoms:

Particularly preferably. L is an aryl group containing 6 to 20 carbon atoms or a heteroaryl group having 3 to 20 carbon atoms;

An aryl group refers to a hydrocarbyl containing at least one aromatic ring, including monocyclic groups and polycyclic ring systems. Heteroaryl groups refer to hydrocarbyl groups (containing heteroatoms) that contain at least one heteroaryl ring, including monocyclic groups and polycyclic ring systems. These polycyclic rings may have two or more rings in which two carbon atoms are shared by two adjacent rings, i.e., a fused ring. At least one of these polycyclic rings is heteroaryl.

In the present disclosure, the aromatic or the heteroaromatic ring systems includes not only aryl or heteroaryl systems, but also a plurality of aryl or heteroaryl may also be interrupted by short non-aromatic units (<10% non-H atoms, preferably less than 5% of non-H atoms, such as C, N, or O atoms). Therefore, systems such as 9, 9′-spirobifluorene, 9, 9-diarylfluorene, triarylamine, diaryl ether, and the like are also considered as aromatic ring systems.

Specifically, the aryl group includes benzene, naphthalene, anthracene, phenanthrene, perylene, tetracene, pyrene, benzopyrene, triphenylene, acenaphthene, fluorene, and derivatives thereof.

Specifically, heteroaryl group includes: furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazonaphthalene, quinoxaline, phenanthridine, primidine, quinazoline, quinazolinone, and derivatives thereof.

Preferably, L is selected from the group consisting of benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, spirosilafluorene and the like.

More preferably, L is selected from the group consisting of benzene, pyridine, pyrimidine, triazine, carbazole, and the like.

Preferably, R₁, R₂ or R₃ is selected from the group consisting of methyl, benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, spirosilafluorene and the like.

More preferably, R₁, R₂ or R₃ is selected from the group consisting of benzene, pyridine, pyrimidine, triazine, carbazole, and the like.

Particularly preferably, L is selected from the following structural units or the units formed by substituting any one of the following structural units:

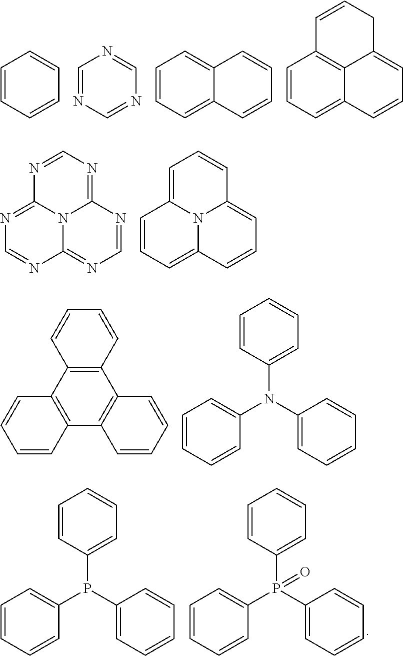

Preferably, A₁, A₂, or A₃ is selected from an aryl group containing 6 to 25 carbon atoms or a heteroaryl group containing 3 to 25 carbon atoms;

More preferably, A₁, A₂, or A₃ is selected from an aryl group containing 6 to 22 carbon atoms or a heteroaryl group containing 3 to 22 carbon atoms;

Particularly preferably, A₁, A₂ or A₃ is one selected from the following structural groups:

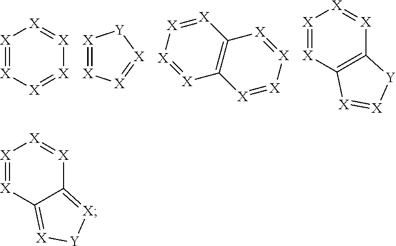

wherein X is selected from CR¹ or N:

Y is selected from CR²R³, SiR²R³, NR², C(═O), S or O;

R¹, R², or R³ is one or a combination of more than one selected from H, D, a linear alkyl group containing 1 to 20 C atoms, an alkoxy group containing 1 to 20 C atoms, a thioalkoxy group containing 1 to 20 C atoms, a branched alkyl group containing 3 to 20 C atoms, a cyclic alkyl group containing 3 to 20 C atoms, an alkoxy or thioalkoxy group containing 3 to 20 C atoms, a silyl group containing 3 to 20 C atoms, a substituted keto group containing 1 to 20 C atoms, a alkoxycarbonyl group containing 2 to 20 C atoms, an aryloxycarbonyl group containing 7 to 20 C atom, a cyano group (—CN), a carbamoyl group (—C(═O)NH₂), a haloformyl group (—C(═O)-A, wherein A represents a halogen atom), a formyl group (—C(═O)—H), an isocyano group, an isocyanate group, a thiocyanate group, an isothiocyanate group, a hydroxyl group, a nitryl group, a CF₃ group, Cl, Br, F, an crosslinkable group, an aromatic ring system containing 5 to 40 ring atoms, a heteroaromatic ring system containing 5 to 40 ring atoms, a substituted aromatic ring system containing 5 to 40 ring atoms, a substituted heteroaromatic ring system containing 5 to 40 ring atoms, an aryloxy group containing 5 to 40 ring atoms, and a heteroaryloxy group containing 5 to 40 ring atoms, wherein one or more of the groups R₁, R₂, and R₃ may form a monocyclic or polycyclic aliphatic or aromatic ring system with each other and/or with a ring bonded to said groups.

In a specific embodiment, A₁, A₂, or A₃ is one selected from the following structural groups or substituted groups formed by one of the following structural groups being further substituted:

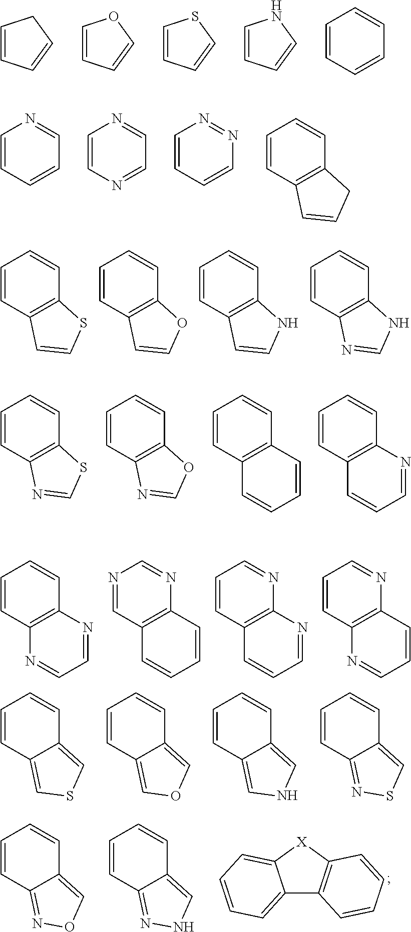

wherein X is selected from the group consisting of N(R), B(R), C(R)₂, Si(R)₂, O, S, C═N(R), C═C(R)₂, P(R), P(═O)R, S═O, SO₂, or X is absent; X is preferably N(R), C(R)₂, O, or S;

R is selected from an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 carbon atoms, and one or more positions of R may be substituted by H, D, F, CN, alkyl, aralkyl, alkenyl, alkynyl, nitrile group, amino, nitro, acyl, alkoxy, carbonyl, sulfonyl group, cycloalkyl, or hydroxy.

In some particularly preferred embodiments, A₁, A₂, or A₃ is one selected from the following structural groups or substituted groups formed by one of the following structural groups being further substituted:

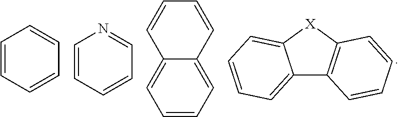

Preferably. R₁, R₂, R₃ or R is selected from the group consisting of methyl, benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, spirosilafluorene and the like.

More preferably, R₁, R₂, R₃ or R is selected from the group consisting of benzene, pyridine, pyrimidine, triazine, carbazole, and the like.

Preferably, the terbenzocyclopentadiene compound disclosed by the present disclosure is one selected from compounds having the following structural formula:

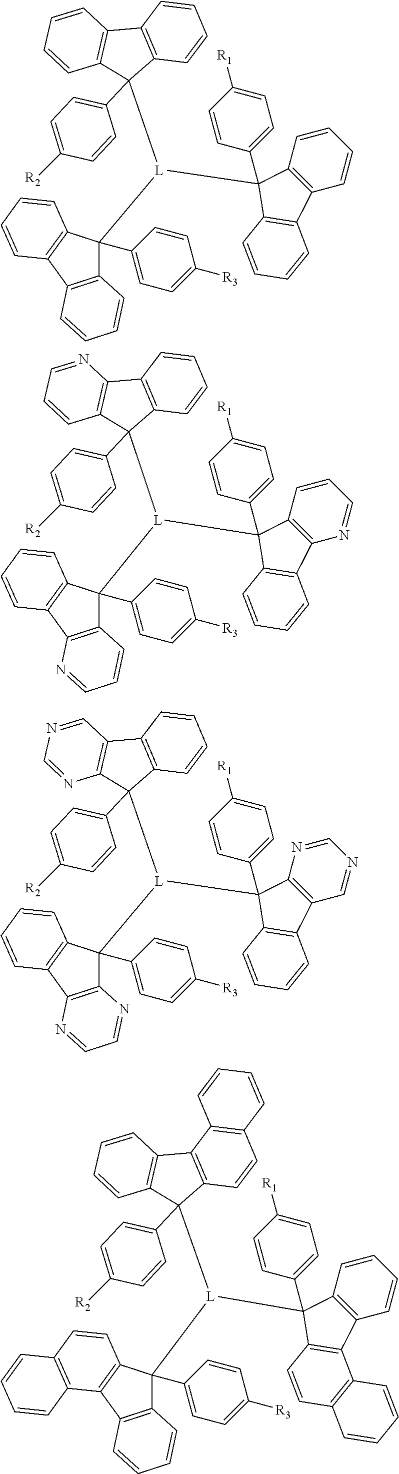

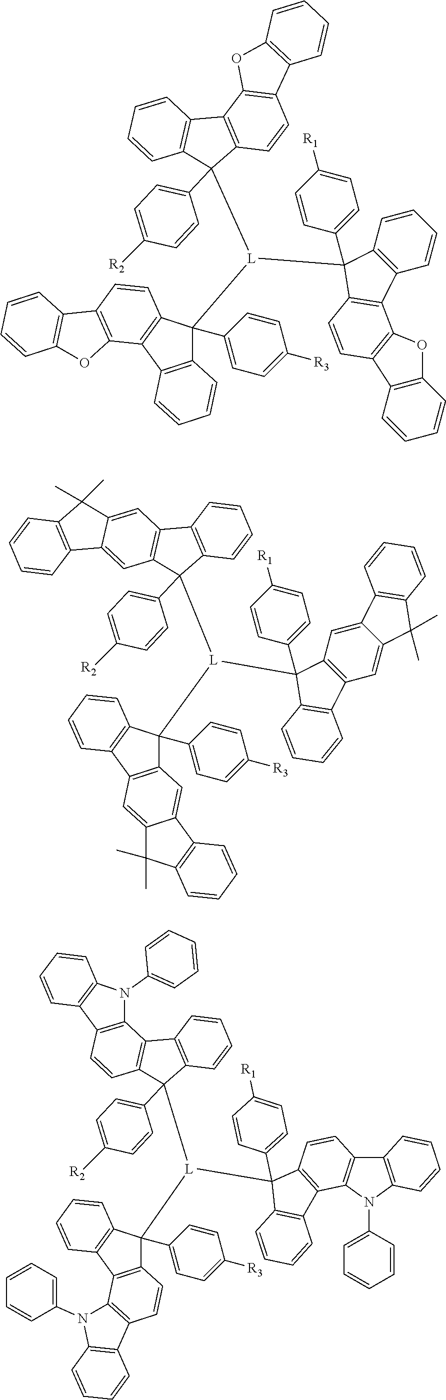

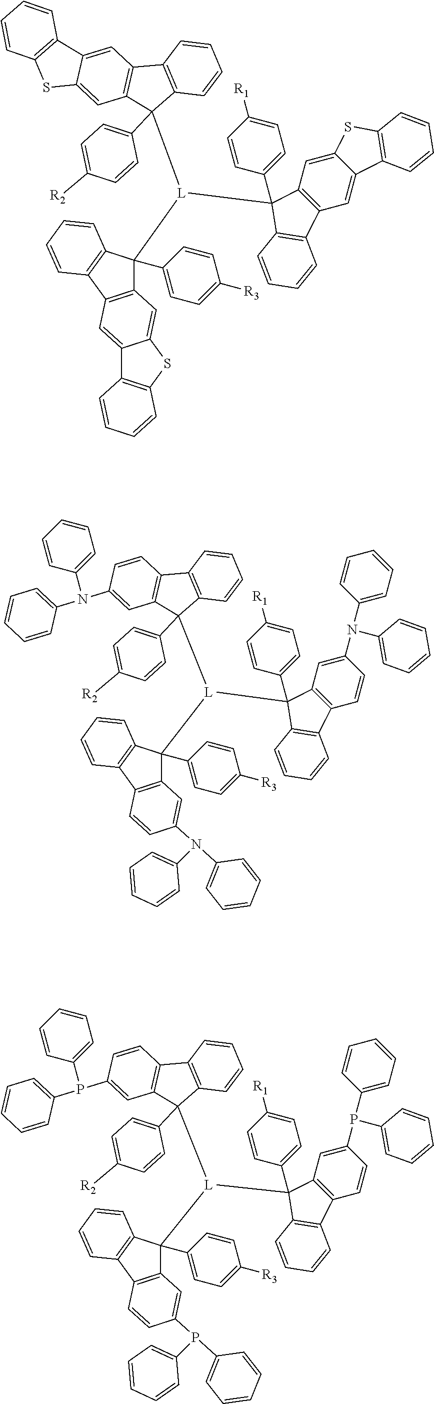

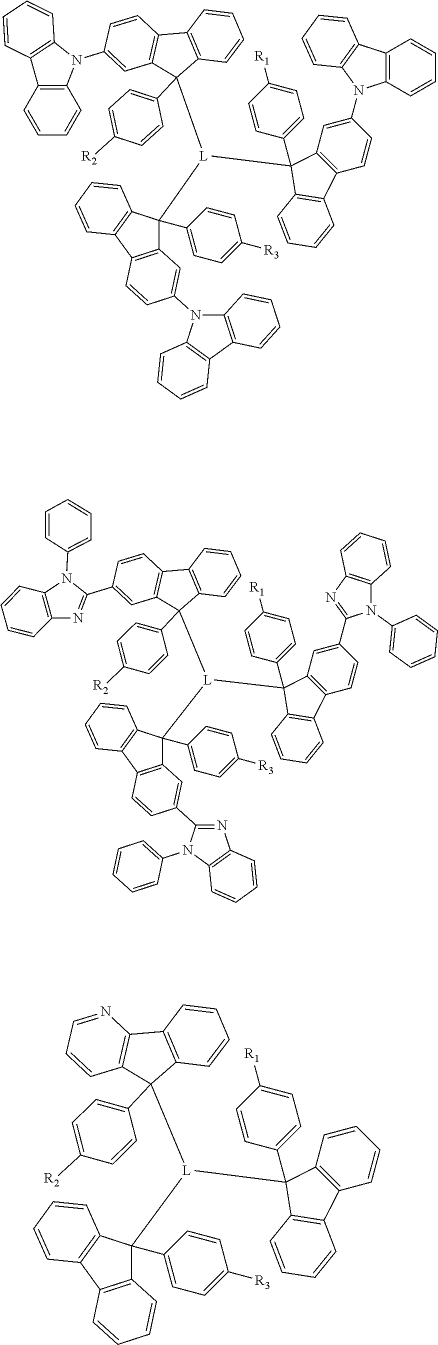

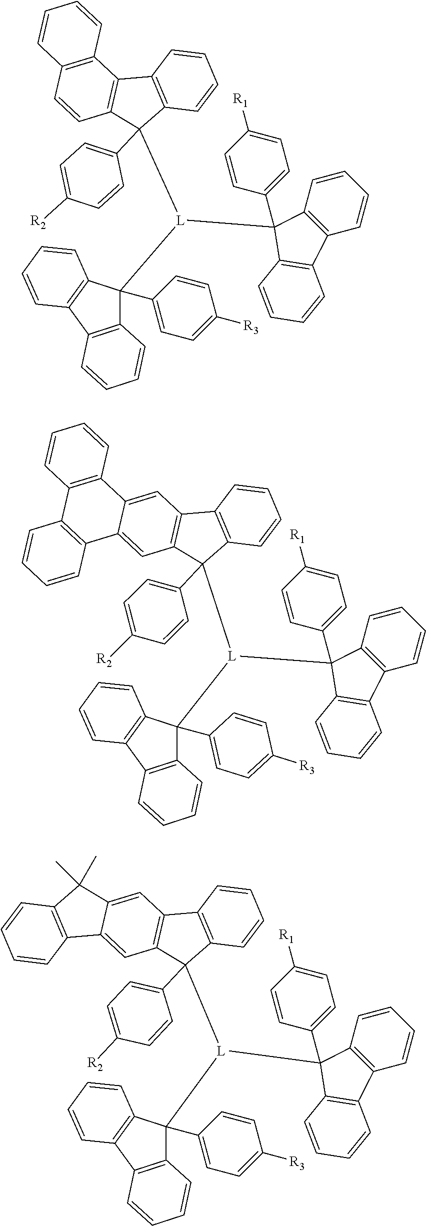

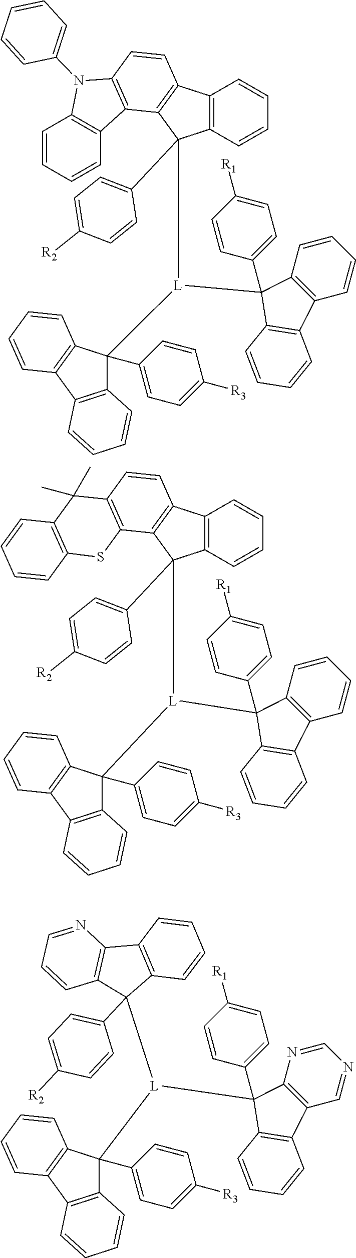

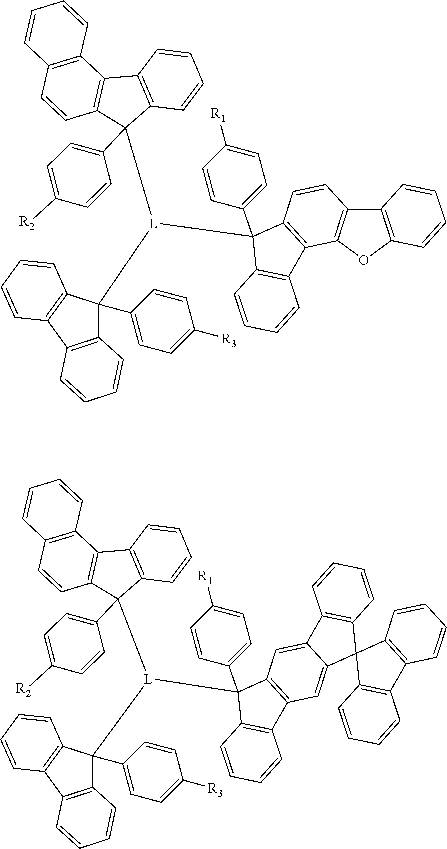

wherein L, R₁, R₂ and R₃ are defined as above.

More preferably, the terbenzocyclopentadiene compound disclosed in the present disclosure is one selected from compounds having the following structural formula:

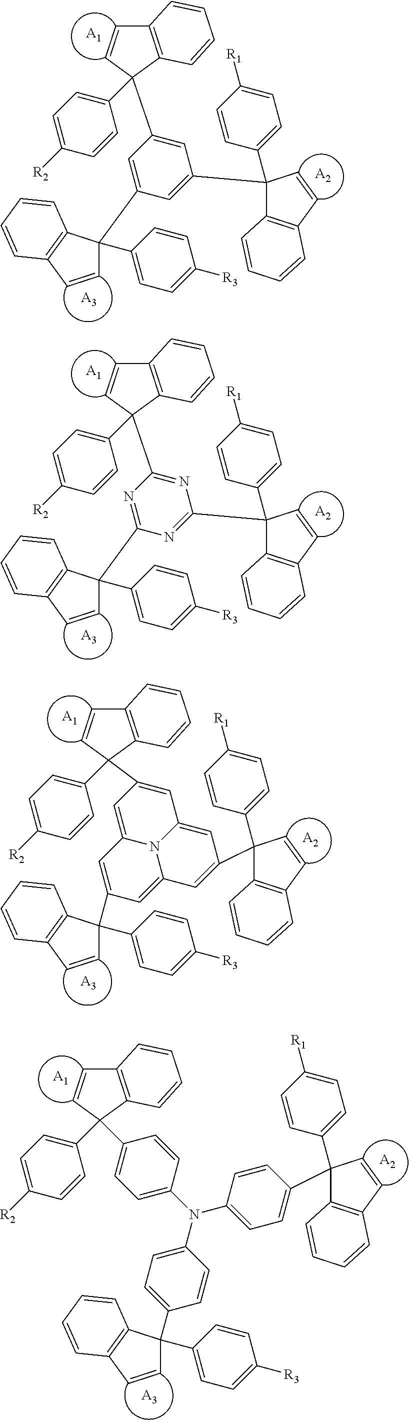

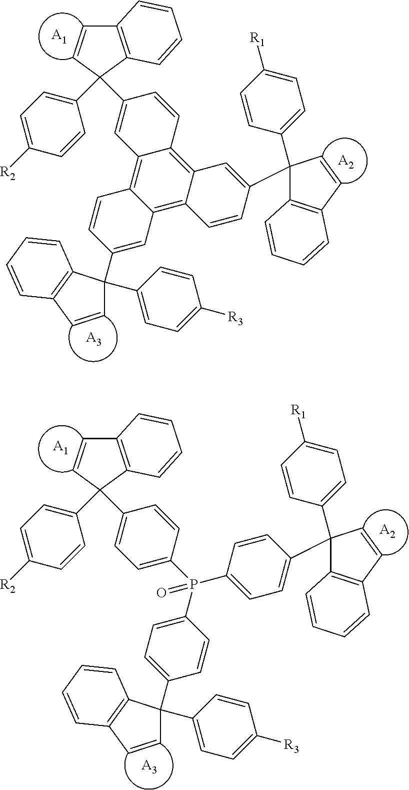

wherein A₁, A₂, A₃, R₁, R₂ and R are defined as above.

The terbenzocyclopentadiene compound disclosed by the present disclosure can be used in electronic devices as a functional material. Organic functional materials include a hole injection material (HIM), a hole transport material (HTM), an electron transport material (ETM), an electron injection material (EIM), an electron blocking material (EBM), a hole blocking material (HBM), the emitters, a host material, and an organic dye.

Preferably, the terbenzocyclopentadiene compound disclosed in the present disclosure can be used as a host material, an electron transport material or a hole transport material.

More preferably, the terbenzocyclopentadiene compound disclosed in the present disclosure can be used as a phosphorescent host material.

The terbenzocyclopentadiene compound, as a phosphorescent host material, must have a proper triplet energy level, i.e., T₁. Preferably, the terbenzocyclopentadiene compound disclosed in the present disclosure has a T₁ greater than or equal to 2.2 eV, preferably greater than or equal to 2.4 eV, more preferably greater than or equal to 2.6 eV, still more preferably greater than or equal to 2.65 eV, and most preferably greater than or equal to 2.7 eV.

In general, the triplet energy level T₁ of the organic compound depends on the substructure having the largest conjugated system in the compound. In general, T₁ decreases as the conjugate system increases.

Preferably, in the general formula (1), one of the substructures represented by the general formula (1a) has the largest conjugated system:

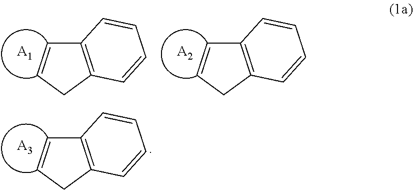

Preferably, in the case where the substituent is removed, ring atoms of the general formula (1a) are no more than 36, preferably no more than 30, more preferably no more than 26, and most preferably no more than 20.

Preferably, according to the substructure of the general formula (1a), T₁ is greater than or equal to 2.3 eV, preferably greater than or equal to 2.5 eV, more preferably greater than or equal to 2.7 eV, and most preferably greater than or equal to 2.75 eV.

A phosphorescent host material is expected to have good thermal stability.

In general, the terbenzocyclopentadiene compound disclosed in the present disclosure has a glass transition temperature Tg greater than or equal to 100° C.

Preferably, the terbenzocyclopentadiene compound disclosed in the present disclosure has a glass transition temperature Tg greater than or equal to 120° C.

More preferably, the terbenzocyclopentadiene compound disclosed in the present disclosure has a glass transition temperature Tg greater than or equal to 140° C.

Still more preferably, the terbenzocyclopentadiene compound disclosed in the present disclosure has a glass transition temperature Tg greater than or equal to 160° C.

Most preferably, the terbenzocyclopentadiene compound disclosed in the present disclosure has a glass transition temperature Tg greater than or equal to 180° C.

In the synthesis of such compounds, an intermediate containing three acyl chloride groups is generally made by a central group L. and then a side group benzene-Rₓ is contained through the Friedel-Crafts reaction; when the central group is prepared, a lithium salt or a Grignard reagent is made from the upper group of the Sp³ carbon atom to attack the carbonyl group of the central group; then a ring-closing reaction is performed so as to yield the target compound.

Specifically, the terbenzocyclopentadiene compound disclosed in the present disclosure is one selected from the following structural formula:

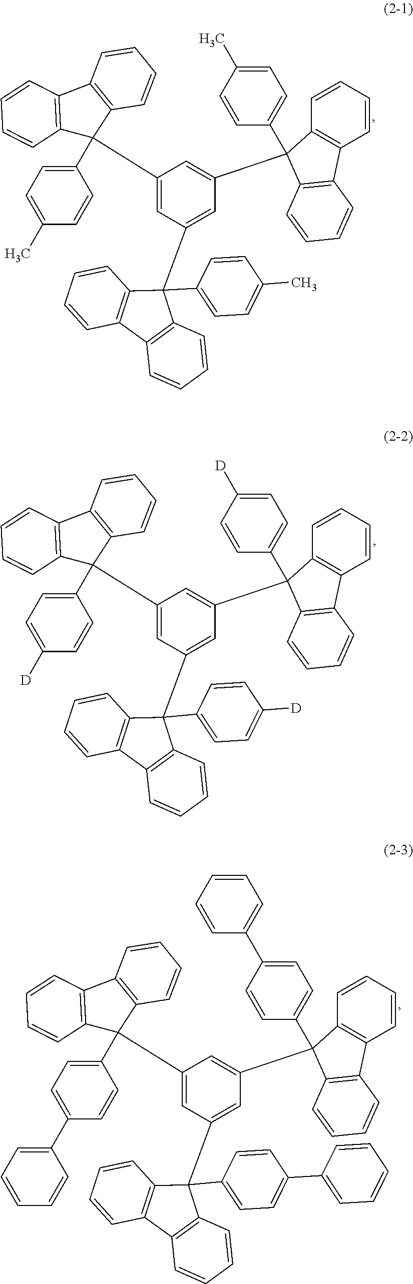

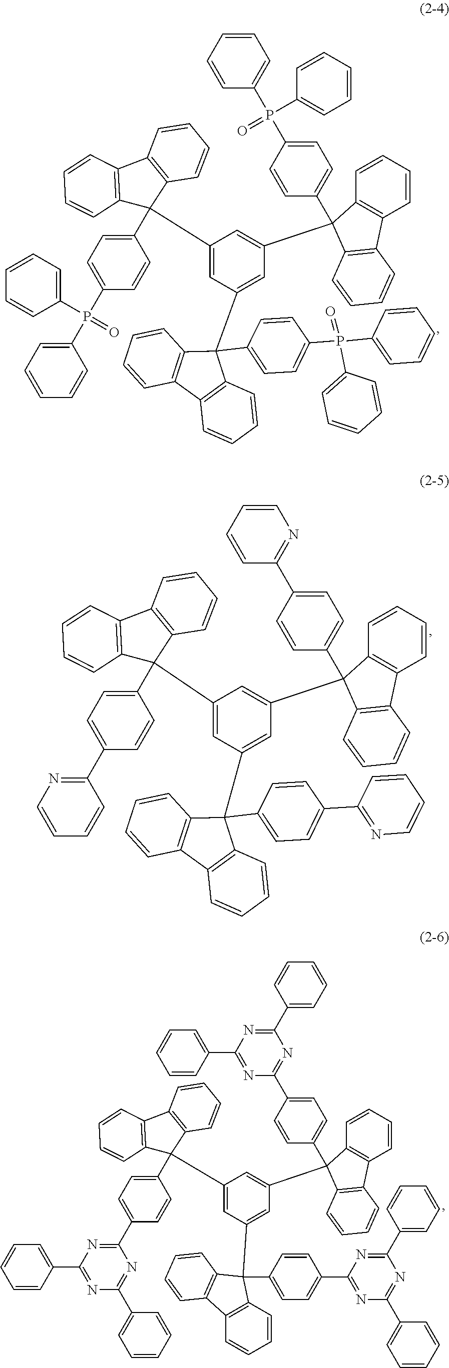

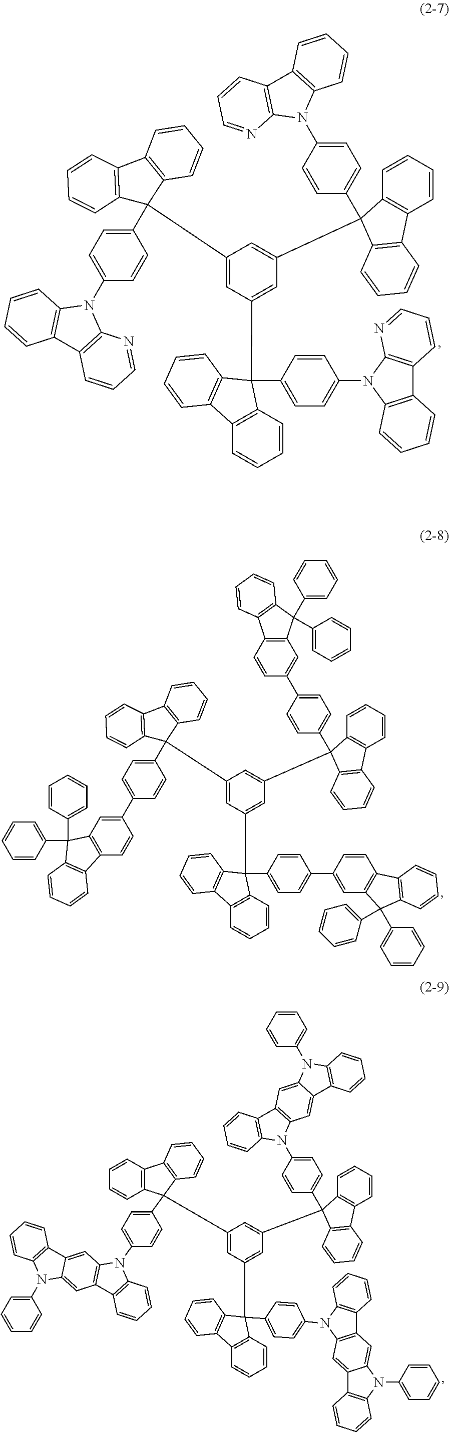

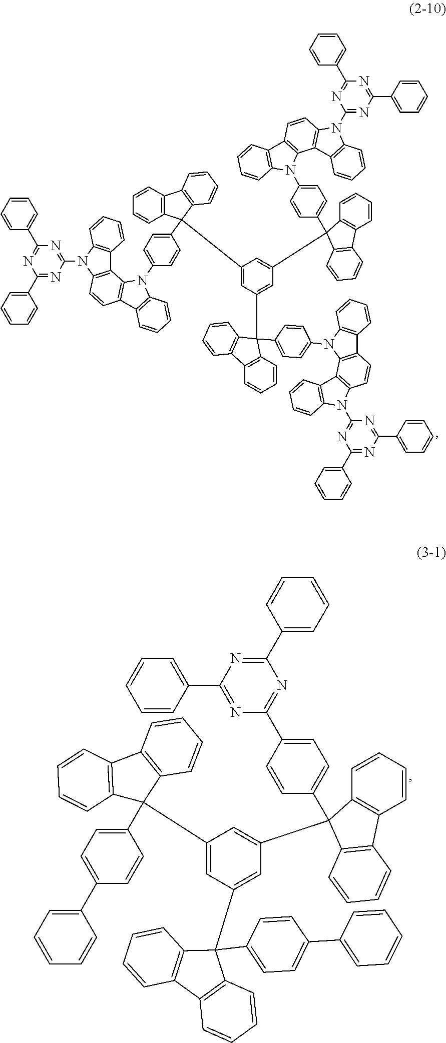

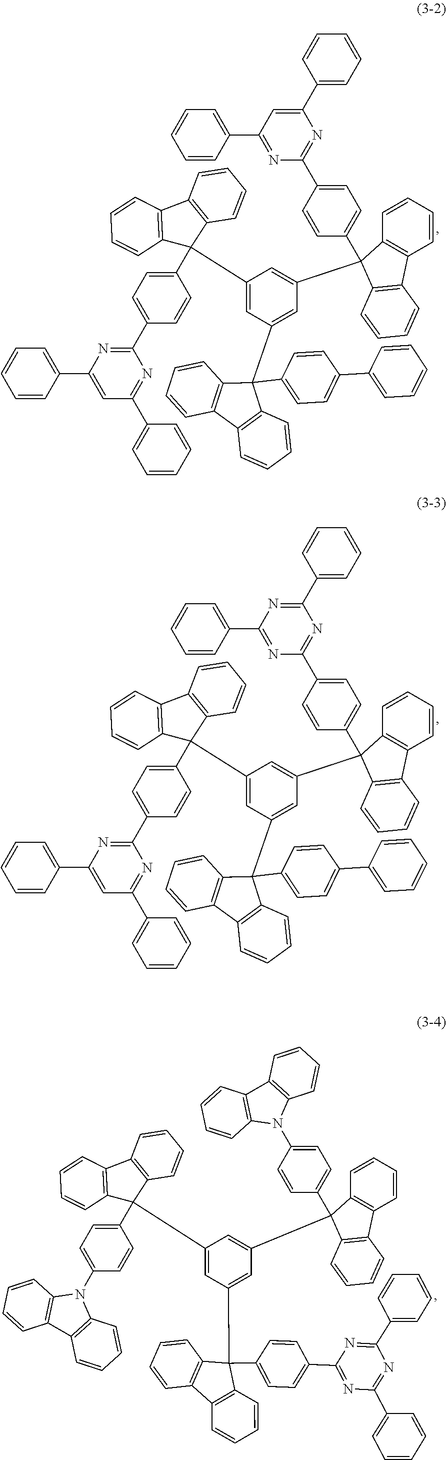

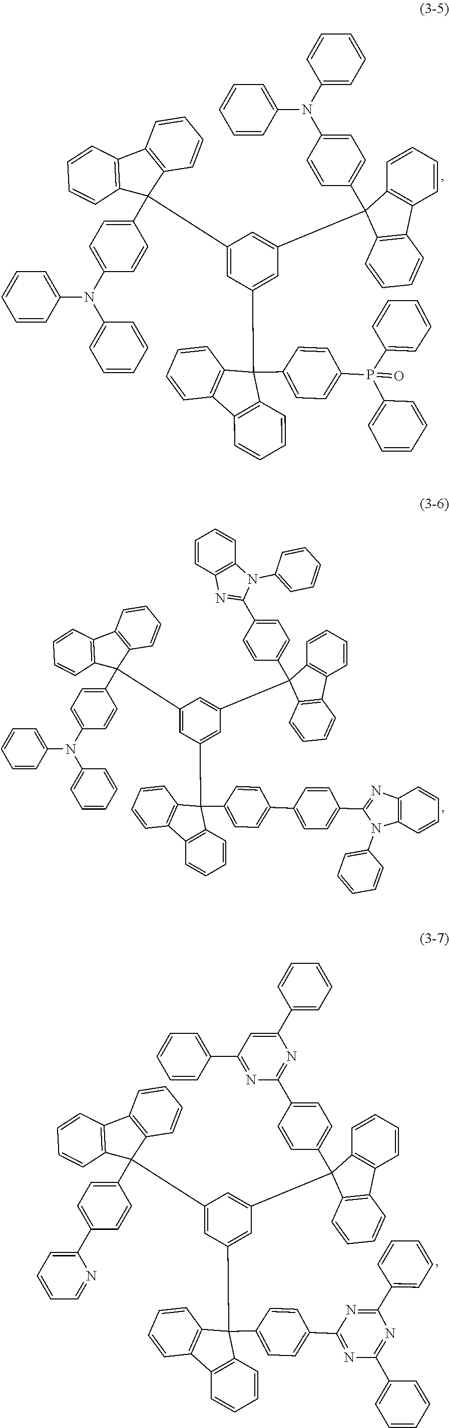

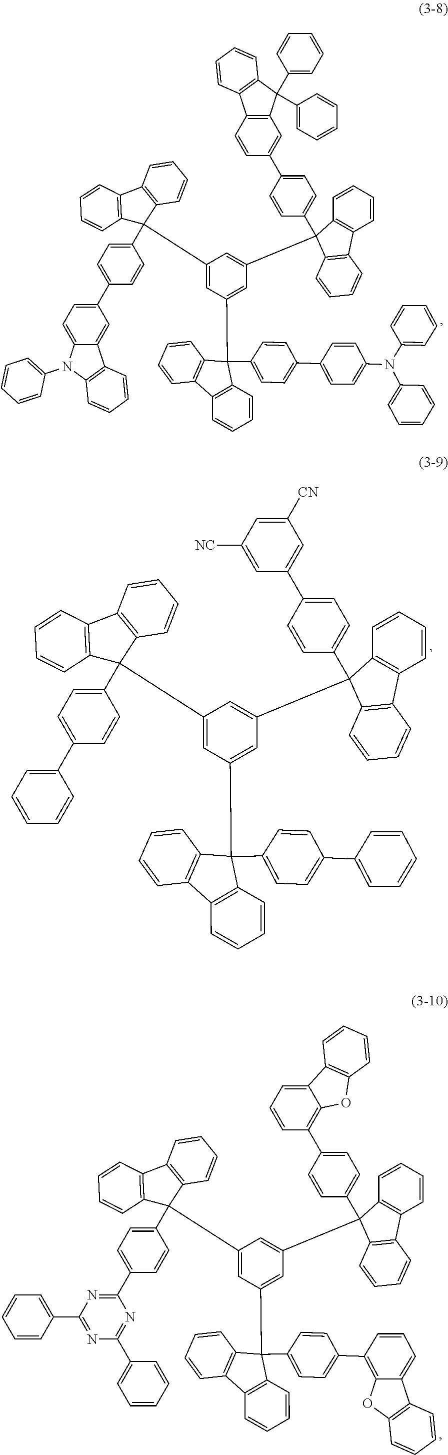

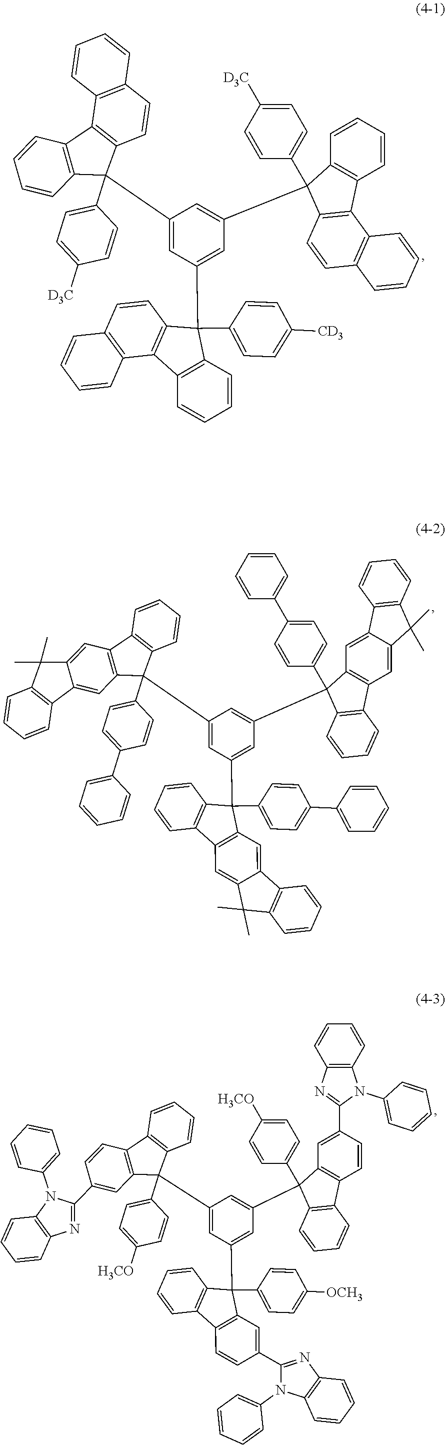

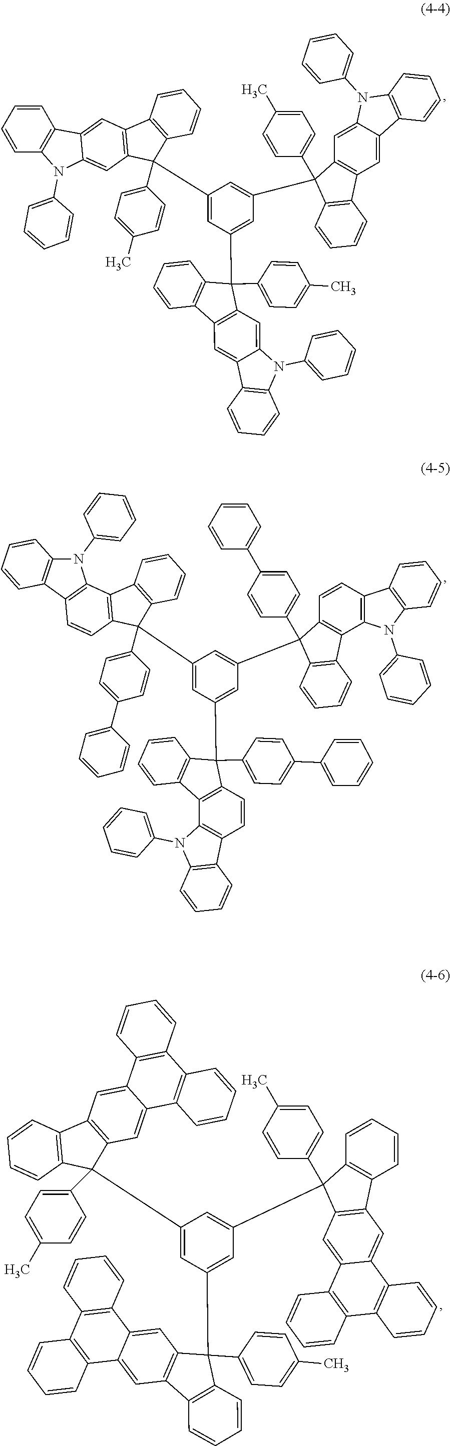

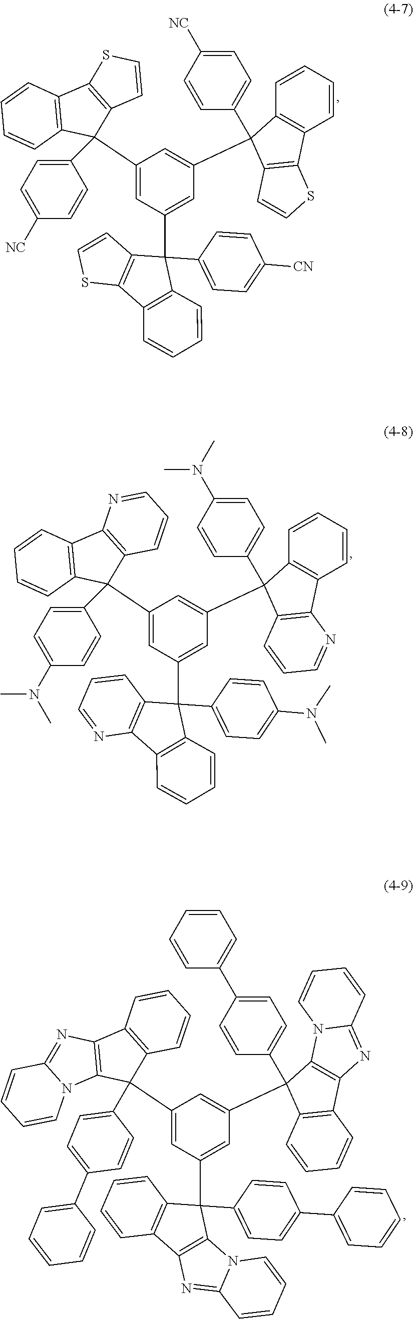 [SMILES: N#Cc1ccc(C2(c3cc(C4(c5ccc(C#N)cc5)c5ccccc5-c5sccc54)cc(C4(c5ccc(C#N)cc5)c5ccccc5-c5sccc54)c3)c3ccccc3-c3sccc32)cc1 ; CN(C)c1ccc(C2(c3cc(C4(c5ccc(N(C)C)cc5)c5ccccc5-c5ncccc54)cc(C4(c5ccc(N(C)C)cc5)c5ccccc5-c5ncccc54)c3)c3ccccc3-c3ncccc32)cc1 ; c1ccc(-c2ccc(C3(c4cc(C5(c6ccc(-c7ccccc7)cc6)c6ccccc6-c6nc7ccccn7c65)cc(C5(c6ccc(-c7ccccc7)cc6)c6ccccc6-c6nc7ccccn7c65)c4)c4ccccc4-c4nc5ccccn5c43)cc2)cc1]

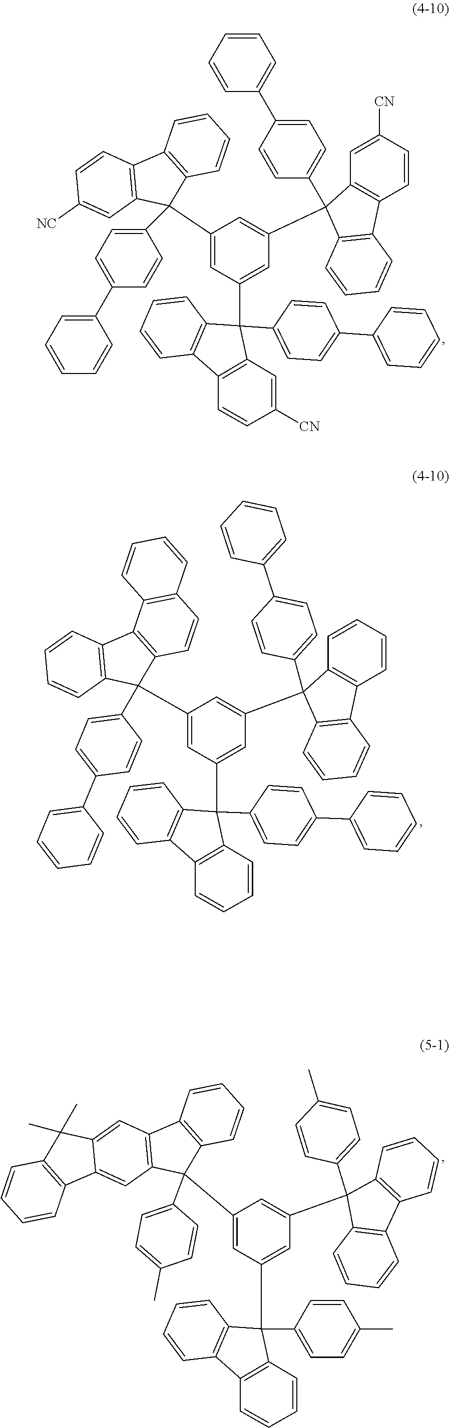

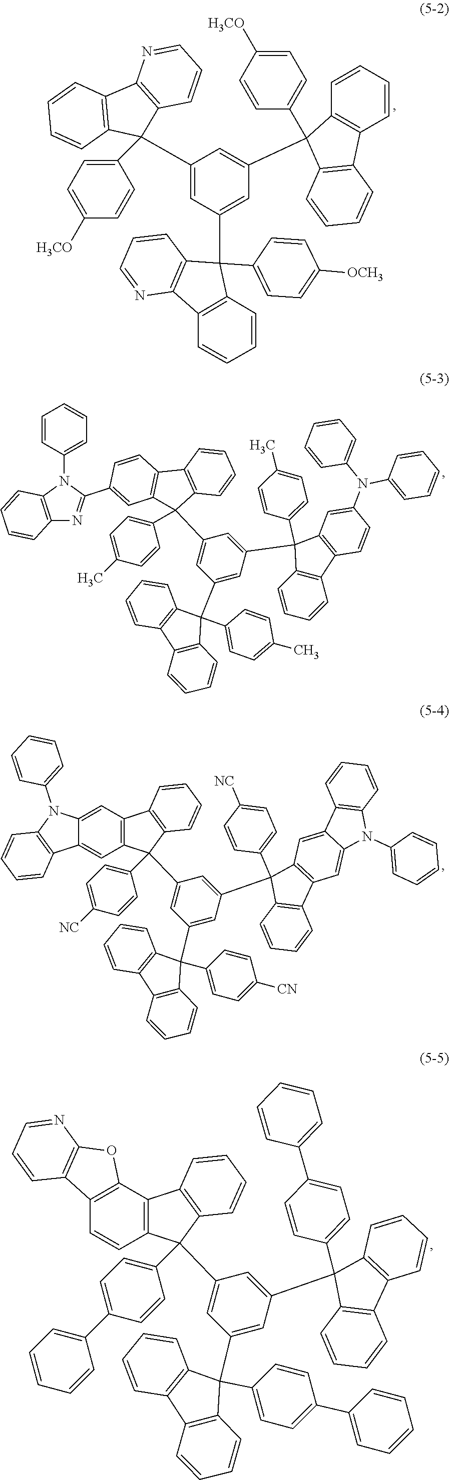

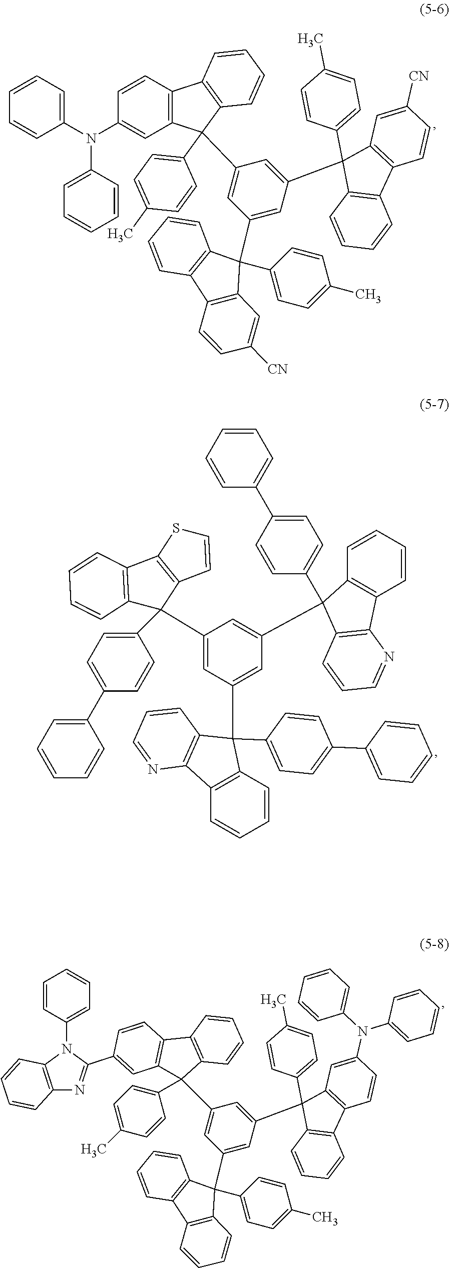 [SMILES: Cc1ccc(C2(c3cc(C4(c5ccc(C)cc5)c5ccccc5-c5ccc(C#N)cc54)cc(C4(c5ccc(C)cc5)c5ccccc5-c5ccc(N(c6ccccc6)c6ccccc6)cc54)c3)c3ccccc3-c3ccc(C#N)cc32)cc1 ; c1ccc(-c2ccc(C3(c4cc(C5(c6ccc(-c7ccccc7)cc6)c6ccccc6-c6ncccc65)cc(C5(c6ccc(-c7ccccc7)cc6)c6ccccc6-c6sccc65)c4)c4ccccc4-c4ncccc43)cc2)cc1 ; Cc1ccc(C2(c3cc(C4(c5ccc(C)cc5)c5ccccc5-c5ccc(-c6nc7ccccc7n6-c6ccccc6)cc54)cc(C4(c5ccc(C)cc5)c5ccccc5-c5ccc(N(c6ccccc6)c6ccccc6)cc54)c3)c3ccccc3-c3ccccc32)cc1]

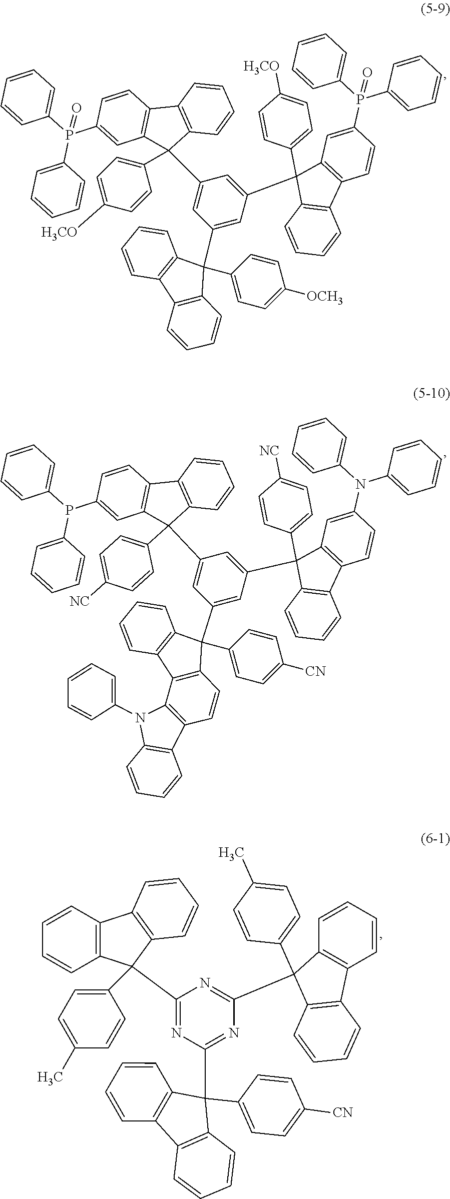 [SMILES: COc1ccc(C2(c3cc(C4(c5ccc(OC)cc5)c5ccccc5-c5ccc(P(=O)(c6ccccc6)c6ccccc6)cc54)cc(C4(c5ccc(OC)cc5)c5ccccc5-c5ccc(P(=O)(c6ccccc6)c6ccccc6)cc54)c3)c3ccccc3-c3ccccc32)cc1 ; N#Cc1ccc(C2(c3cc(C4(c5ccc(C#N)cc5)c5ccccc5-c5ccc(P(c6ccccc6)c6ccccc6)cc54)cc(C4(c5ccc(C#N)cc5)c5ccccc5-c5c4ccc4c6ccccc6n(-c6ccccc6)c54)c3)c3ccccc3-c3ccc(N(c4ccccc4)c4ccccc4)cc32)cc1 ; Cc1ccc(C2(c3nc(C4(c5ccc(C)cc5)c5ccccc5-c5ccccc54)nc(C4(c5ccc(C#N)cc5)c5ccccc5-c5ccccc54)n3)c3ccccc3-c3ccccc32)cc1]

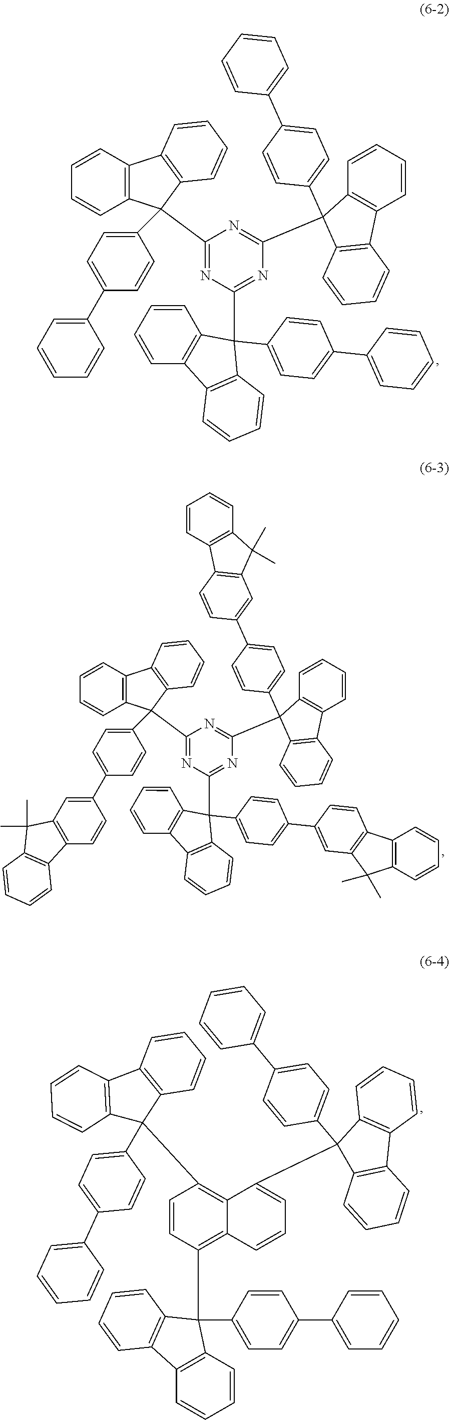

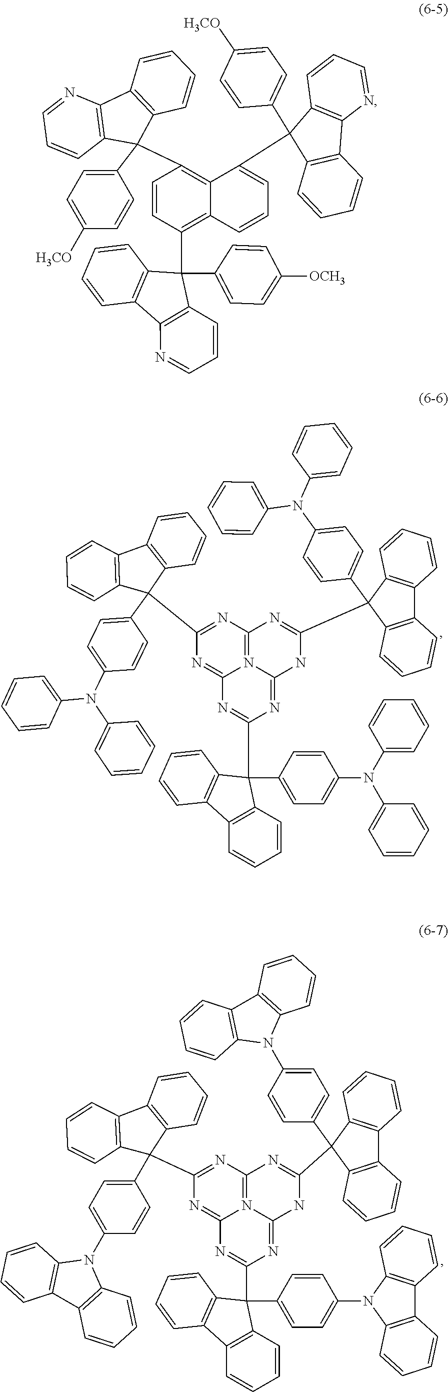

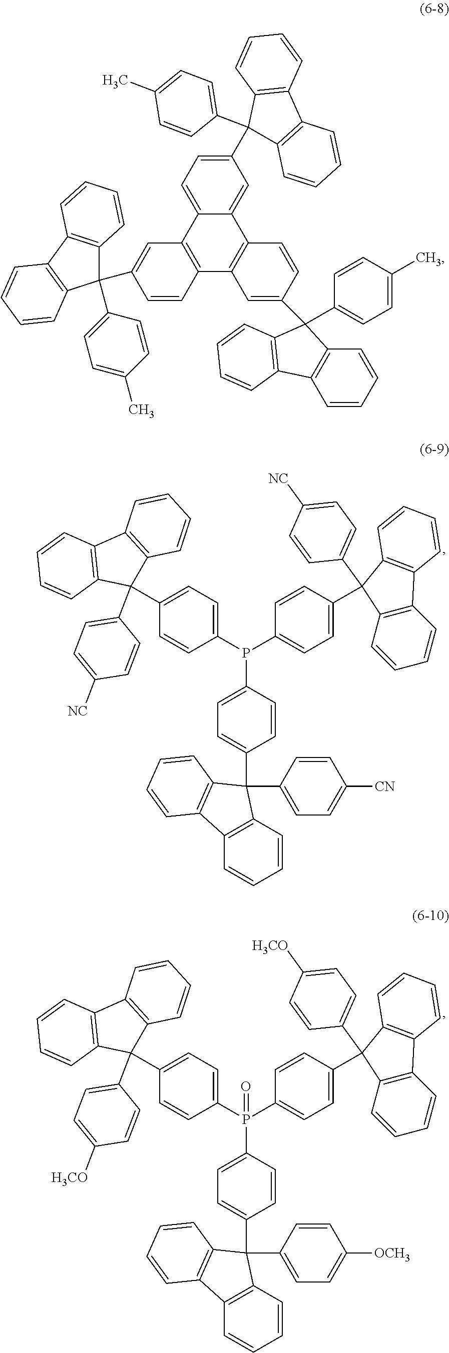

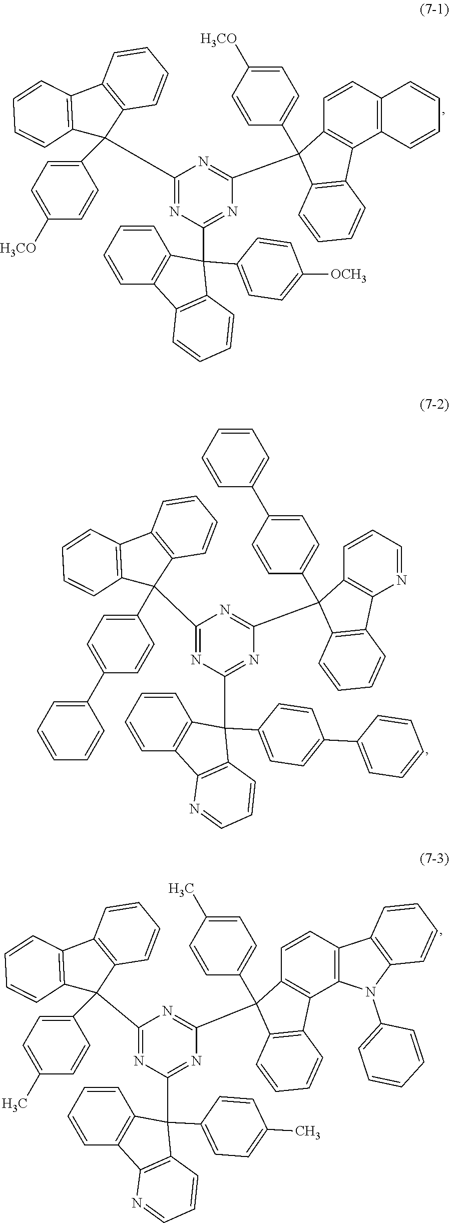 [SMILES: COc1ccc(C2(c3nc(C4(c5ccc(OC)cc5)c5ccccc5-c5ccccc54)nc(C4(c5ccc(OC)cc5)c5ccccc5-c5c4ccc4ccccc54)n3)c3ccccc3-c3ccccc32)cc1 ; c1ccc(-c2ccc(C3(c4nc(C5(c6ccc(-c7ccccc7)cc6)c6ccccc6-c6ncccc65)nc(C5(c6ccc(-c7ccccc7)cc6)c6ccccc6-c6ncccc65)n4)c4ccccc4-c4ccccc43)cc2)cc1 ; Cc1ccc(C2(c3nc(C4(c5ccc(C)cc5)c5ccccc5-c5ncccc54)nc(C4(c5ccc(C)cc5)c5ccccc5-c5c4ccc4c6ccccc6n(-c6ccccc6)c54)n3)c3ccccc3-c3ccccc32)cc1]

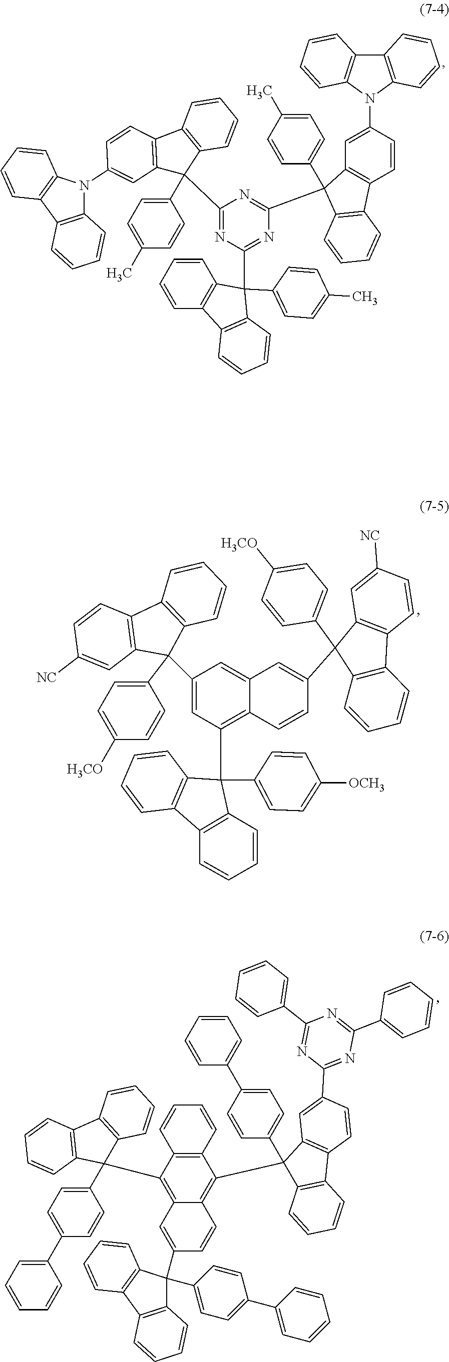 [SMILES: Cc1ccc(C2(c3nc(C4(c5ccc(C)cc5)c5ccccc5-c5ccc(-n6c7ccccc7c7ccccc76)cc54)nc(C4(c5ccc(C)cc5)c5ccccc5-c5ccc(-n6c7ccccc7c7ccccc76)cc54)n3)c3ccccc3-c3ccccc32)cc1 ; COc1ccc(C2(c3ccc4c(C5(c6ccc(OC)cc6)c6ccccc6-c6ccccc65)cc(C5(c6ccc(OC)cc6)c6ccccc6-c6ccc(C#N)cc65)cc4c3)c3ccccc3-c3ccc(C#N)cc32)cc1 ; c1ccc(-c2ccc(C3(c4ccc5c(C6(c7ccc(-c8ccccc8)cc7)c7ccccc7-c7ccc(-c8nc(-c9ccccc9)nc(-c9ccccc9)n8)cc76)c6ccccc6c(C6(c7ccc(-c8ccccc8)cc7)c7ccccc7-c7ccccc76)c5c4)c4ccccc4-c4ccccc43)cc2)cc1]

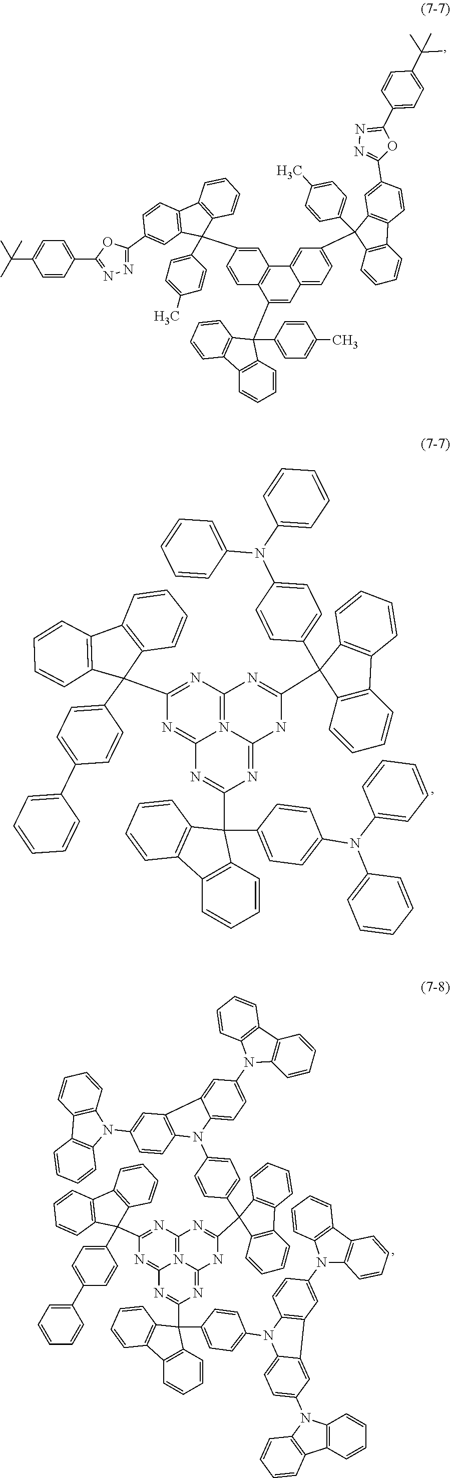

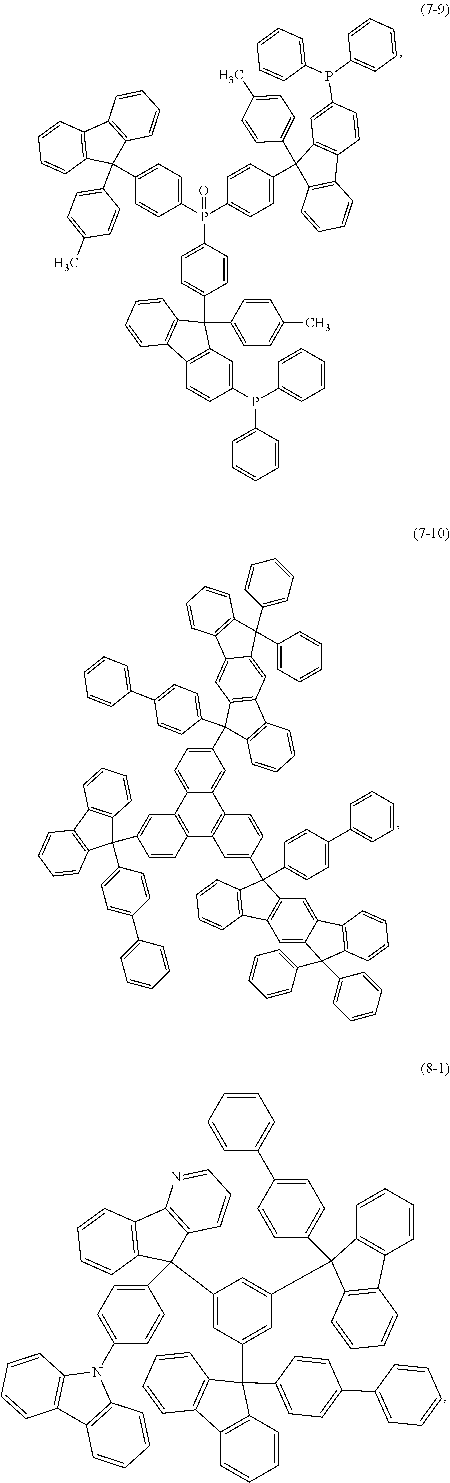

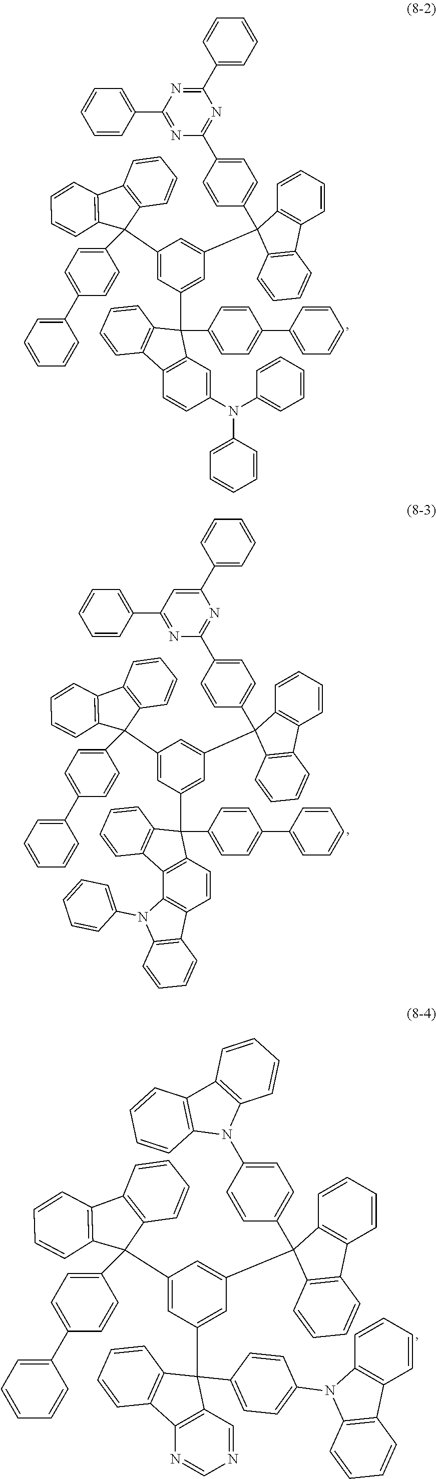

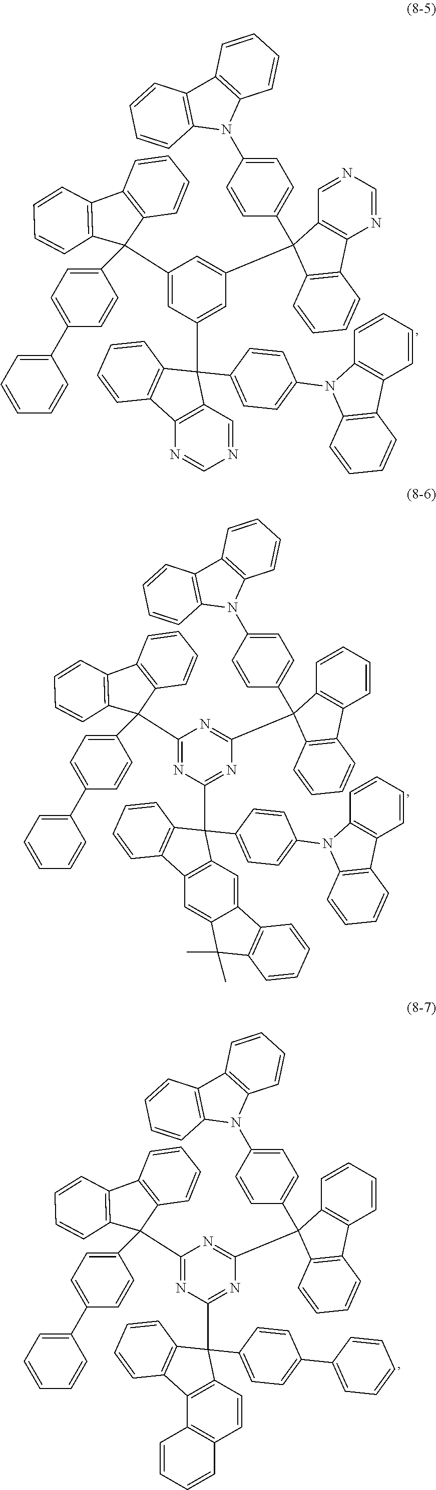 [SMILES: c1ccc(-c2ccc(C3(c4cc(C5(c6ccc(-n7c8ccccc8c8ccccc87)cc6)c6ccccc6-c6ncncc65)cc(C5(c6ccc(-n7c8ccccc8c8ccccc87)cc6)c6ccccc6-c6ncncc65)c4)c4ccccc4-c4ccccc43)cc2)cc1 ; CC1(C)c2ccccc2-c2cc3c(cc21)-c1ccccc1C3(c1ccc(-n2c3ccccc3c3ccccc32)cc1)c1nc(C2(c3ccc(-c4ccccc4)cc3)c3ccccc3-c3ccccc32)nc(C2(c3ccc(-n4c5ccccc5c5ccccc54)cc3)c3ccccc3-c3ccccc32)n1 ; c1ccc(-c2ccc(C3(c4nc(C5(c6ccc(-n7c8ccccc8c8ccccc87)cc6)c6ccccc6-c6ccccc65)nc(C5(c6ccc(-c7ccccc7)cc6)c6ccccc6-c6c5ccc5ccccc65)n4)c4ccccc4-c4ccccc43)cc2)cc1]

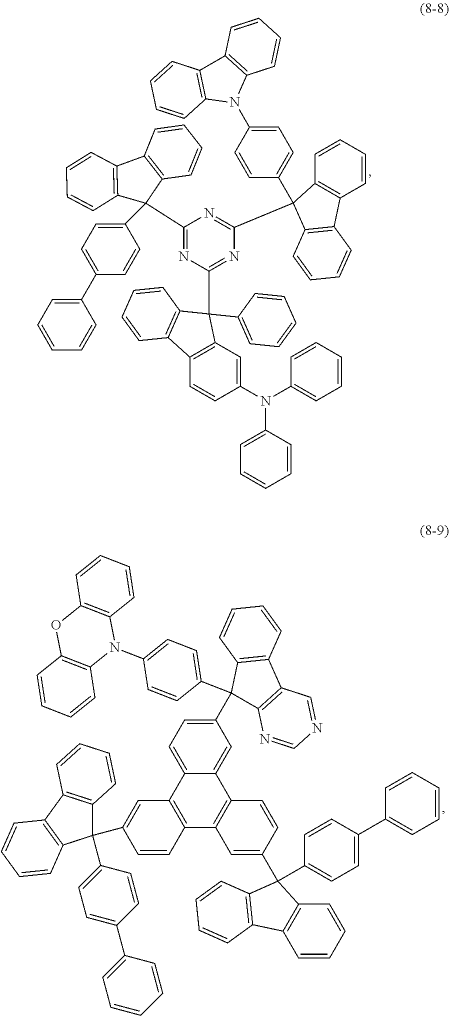 [SMILES: c1ccc(-c2ccc(C3(c4nc(C5(c6ccc(-n7c8ccccc8c8ccccc87)cc6)c6ccccc6-c6ccccc65)nc(C5(c6ccccc6)c6ccccc6-c6ccc(N(c7ccccc7)c7ccccc7)cc65)n4)c4ccccc4-c4ccccc43)cc2)cc1 ; c1ccc(-c2ccc(C3(c4ccc5c(c4)c4ccc(C6(c7ccc(-c8ccccc8)cc7)c7ccccc7-c7ccccc76)cc4c4ccc(C6(c7ccc(N8c9ccccc9Oc9ccccc98)cc7)c7ccccc7-c7cncnc76)cc54)c4ccccc4-c4ccccc43)cc2)cc1]

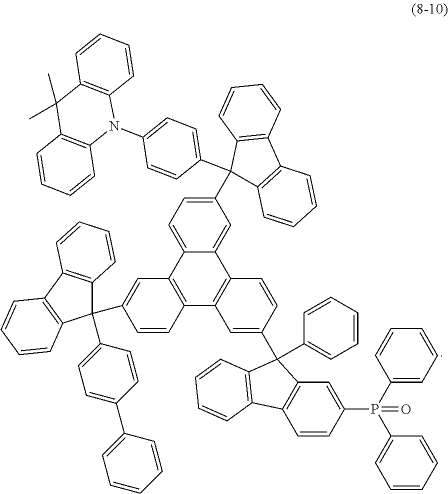 [SMILES: CC1(C)c2ccccc2N(c2ccc(C3(c4ccc5c(c4)c4ccc(C6(c7ccccc7)c7ccccc7-c7ccc(P(=O)(c8ccccc8)c8ccccc8)cc76)cc4c4ccc(C6(c7ccc(-c8ccccc8)cc7)c7ccccc7-c7ccccc76)cc54)c4ccccc4-c4ccccc43)cc2)c2ccccc21]

Preferably, the terbenzocyclopentadiene compound disclosed in the present disclosure is a small molecule material.

As used herein, the term “small molecule” refers to a molecule that is not a polymer, oligomer, dendrimer, or blend. In particular, there is no repetitive structure in small molecules. The molecular weight of the small molecule is no greater than 3000 g/mole, more preferably no greater than 2000 g/mole, and most preferably no greater than 1500 g/mole.

Polymer includes homopolymer, copolymer, and block copolymer. In addition, in the present disclosure, the polymer also includes dendrimer. The synthesis and application of dendrimers are described in Dendrimers and Dendrons, Wiley-VCH Verlag GmbH & Co. KGaA, 2002, Ed. George R. Newkome, Charles N. Moorefield, Fritz Vogtle.

Conjugated polymer is a polymer whose backbone is primarily consisted of the sp2 hybrid orbital of carbon (C) atom. Some known examples are polyacetylene and poly (phenylene vinylene), on the backbone of which the C atom can also be optionally substituted by other non-C atoms, and which is still considered to be a conjugated polymer when the sp2 hybridization on the backbone is interrupted by some natural defects. In addition, the conjugated polymer in the present disclosure may also comprise aryl amine, aryl phosphine and other heteroarmotics, organometallic complexes, and the like on the backbone.

The present disclosure also relates to a polymer comprising at least the above repeating unit having the general formula (1).

Preferably, the polymer is a non-conjugated polymer, and a terbenzocyclopentadiene structural unit having the general formula (1) is situated at a side chain of the polymer.

Preferably, the polymer is a conjugated polymer.

The disclosure also relates to a mixture comprising the above terbenzocyclopentadiene compound or the above polymer, and an organic functional material.

The organic functional material includes: a hole (also called an electron hole) injection or transport material (HIM/HTM), a hole blocking material (HBM), an electron injection or transport material (EIM/ETM), an electron blocking material (EBM), an organic host material (Host), a singlet emitter (fluorescent emitter), a triplet emitter (phosphorescent emitter), in particular, organic emitting metal complexes, and organic dyes. Various organic functional materials are described in detail in, for example, WO2010135519A1, US20090134784A1, and WO2011110277A1, the entire contents of which are hereby incorporated by reference.

The organic functional material may be selected from a small molecule and a polymer material.

The mixture has the terbenzocyclopentadiene compound in an amount of 50 wt % to 99.9 wt %, preferably 60 wt % to 97 wt %, more preferably 70 wt % to 95 wt %, and most preferably 70 wt % to 90 wt %.

Preferably, the mixture comprises the above terbenzocyclopentadiene compound or the above polymer, and a phosphorescent emitting material.

Preferably, the mixture comprises the above terbenzocyclopentadiene compound or the above polymer, and a TADF material.

Preferably, the mixture comprises the above terbenzocyclopentadiene compound or the above polymer, a phosphorescent emitting material and a TADF material.

Preferably, the mixture comprises the above terbenzocyclopentadiene compound or the above polymer, and a fluorescent emitting material.

Preferably, the mixture comprises the above terbenzocyclopentadiene compound or the above polymer, and a light-emitting quantum dot.

The fluorescent emitting material or singlet emitter, phosphorescent emitting material or triplet emitter, TADF material, and light-emitting quantum dot are described in more detail below (but not limited thereto).

1. Singlet Emitter

The singlet emitter tends to have a longer conjugate i-electron system. To date, there have been many examples, such as, but not limited to, styrylamine and derivatives thereof disclosed in JP2913116B and WO2001021729A1, and indenofluorene and derivatives thereof disclosed in WO2008/006449 and WO2007/140847.

Preferably, the singlet emitter may be selected from the group consisting of monostyrylamines, distyrylamines, tristyrylamines, tetrastyrylamines, styrylphosphines, styryl ethers, and arylamines.

Mono styrylamine refers to a compound which comprises an unsubstituted or optionally substituted styryl group and at least one amine, most preferably an aromatic amine.

Distyrylamine refers to a compound comprising two unsubstituted or optionally substituted styryl groups and at least one amine, most preferably an aromatic amine.

Ternarystyrylamine refers to a compound which comprises three unsubstituted or optionally substituted styryl groups and at least one amine, most preferably an aromatic amine.

Quatemarystyrylamine refers to a compound comprising four unsubstituted or optionally substituted styryl groups and at least one amine, most preferably an aromatic amine.

Preferred styrene is stilbene, which may be further optionally substituted.

The corresponding phosphines and ethers are defined similarly to amines.

Aryl amine or aromatic amine refers to a compound comprising three unsubstituted or optionally substituted aromatic cyclic or heterocyclic systems directly attached to nitrogen. At least one of these aromatic cyclic or heterocyclic systems is preferably selected from fused ring systems and most preferably has at least 14 aromatic ring atoms. Among the preferred examples are selected from aromatic anthramine, aromatic anthradiamine, aromatic pyrene amines, aromatic pyrene diamines, aromatic chrysene amines and aromatic chrysene diamine.

Aromatic anthramine refers to a compound in which a diarylamino group is directly attached to anthracene, particularly at position 9.

Aromatic anthradiamine refers to a compound in which two diarylamino groups are directly attached to anthracene, particularly at positions 9, 10.

Aromatic pyrene amines, aromatic pyrene diamines, aromatic chrysene amines and aromatic chrysene diamine are similarly defined, wherein the diarylarylamino group is particularly attached to position 1 or 1 and 6 of pyrene.

Examples of singlet emitter based on vinylamine and arylamine are also preferred examples which may be found in the following patent documents: WO 2006/000388, WO 2006/058737, WO 2006/000389, WO 2007/065549, WO 2007/115610, U.S. Pat. No. 7,250,532 B2. DE 102005058557 A1, CN 1583691 A, JP 08053397 A, U.S. Pat. No. 6,251,531 B1, US 2006/210830 A, EP 1957606 A1, and US 2008/0113101 A1, the whole contents of which are incorporated herein by reference.

Examples of singlet light emitters based on distyrylbenzene and its derivatives may be found in, for example, U.S. Pat. No. 5,121,029.

Further preferred singlet emitters may be selected from the group consisting of: indenofluorene-amine and indenofluorene-diamine such as disclosed in WO 2006/122630, benzoindenofluorene-amine and benzoindenofluorene-diamine such as disclosed in WO 2008/006449, dibenzoindenofluorene-amine and dibenzoindenofluorene-diamine such as disclosed in WO2007/140847.

Other materials useful as singlet emitters include, but not limited to, polycyclic aromatic compounds, especially any one selected from the derivatives of the following compounds: anthracenes such as 9,10-di-naphthylanthracene, naphthalene, tetraphenyl, oxyanthene, phenanthrene, perylene (such as 2,5,8,11-tetra-t-butylatedylene), indenoperylene, phenylenes (such as 4,4′-(bis (9-ethyl-3-carbazovinylene)-1,1′-biphenyl), periflanthene, decacyclene, coronene, fluorene, spirobifluorene, arylpyren (e.g., US20060222886), arylenevinylene (e.g., U.S. Pat. No. 5,121,029, U.S. Pat. No. 5,130,603), cyclopentadiene such as tetraphenylcyclopentadiene, rubrene, coumarine, rhodamine, quinacridone, pyrane such as 4 (dicyanocthylene)-6-(4-dimethylaminostyryl-2-methyl)-4H-pyrane (DCM), thiapyran, bis (azinyl) imine-boron compounds (US 2007/0092753 A1), bis (azinyl) methene compounds, carbostyryl compounds, oxazone, benzoxazole, benzothiazole, benzimidazole, and diketopyrrolopyrrole. Examples of some singlet emitter materials may be found in the following patent documents: US 20070252517 A1, U.S. Pat. No. 4,769,292, U.S. Pat. No. 6,020,078. US 2007/0252517 A1. US 2007/0252517 A1, the whole contents of which are incorporated herein by reference.

Examples of suitable singlet emitters are listed below:

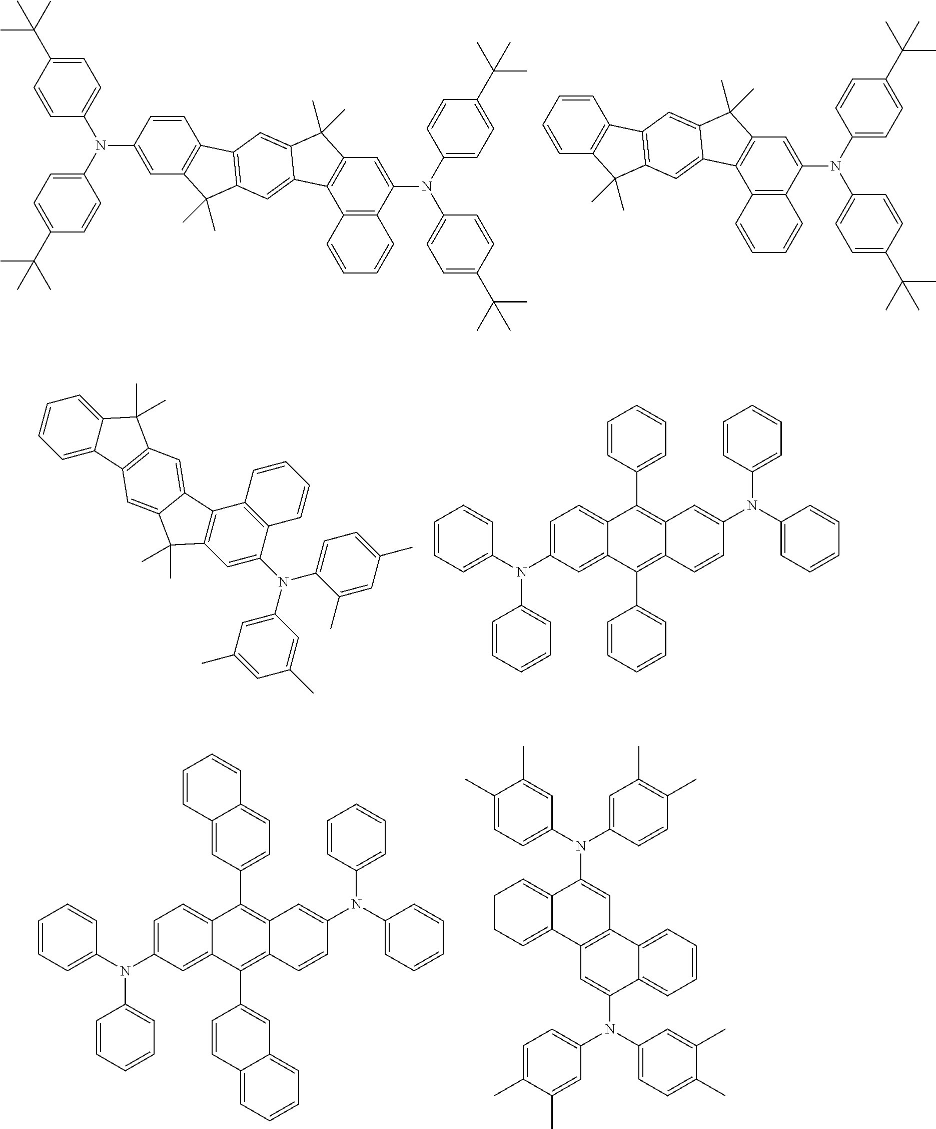

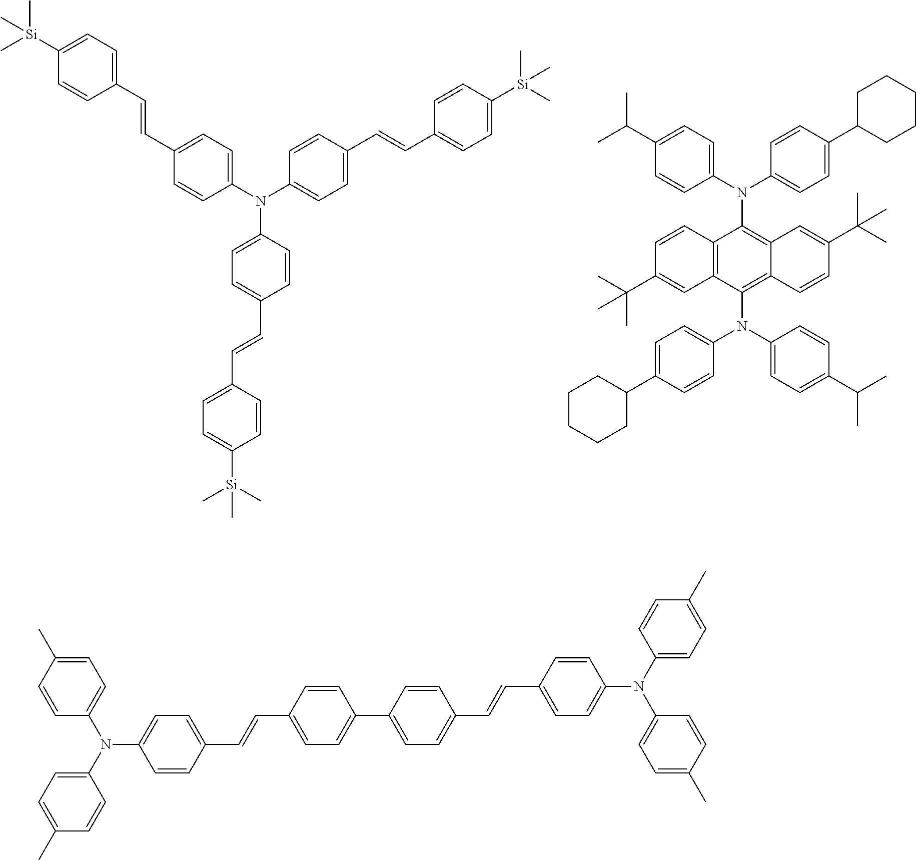

2. Thermally Activated Delayed Fluorescent Material (TADF):

Traditional organic fluorescent materials can only emit light using 25% singlet excitonic luminescence formed by electrical excitation, and the devices have relatively low internal quantum efficiency (up to 25%). The phosphorescent material enhances the intersystem crossing due to the strong spin-orbit coupling of the heavy atom center, the singlet exciton and the triplet exciton luminescence formed by the electric excitation can be effectively utilized, so that the internal quantum efficiency of the device can reach 100%. However, the phosphor materials are expensive, the material stability is poor, and the device efficiency roll-off is a serious problem, which limit its application in OLED. Thermally-activated delayed fluorescent materials are the third generation of organic light-emitting materials developed after organic fluorescent materials and organic phosphorescent materials. This type of material generally has a small singlet-triplet energy level difference (ΔEst), and triplet excitons can be converted to singlet excitons by intersystem crossing. This can make full use of the singlet excitons and triplet excitons formed under electric excitation. The device can achieve 100% quantum efficiency.

The TADF material needs to have a small singlet-triplet energy level difference, typically ΔEst<0.3 eV, preferably ΔEst<0.2 eV, more preferably ΔEst<0.1 eV, and most preferably ΔEst<0.05 eV. In a preferred embodiment, TADF has good fluorescence quantum efficiency. Some TADF emitting materials can be found in the following patent documents: CN103483332(A). TW201309696(A), TW201309778(A). TW201343874(A). TW201350558(A), US20120217869(A1), WO2013133359(A1), WO2013154064 (A1), Adachi, et. al. Adv. Mater., 21, 2009, 4802, Adachi, et. al. Appl. Phys. Lett., 98, 2011, 083302. Adachi, et. al. Appl. Phys. Lett., 101, 2012, 093306, Adachi, et. al. Chem. Commun., 48, 2012, 11392, Adachi, et. al. Nature Photonics, 6, 2012, 253, Adachi, et. al. Nature, 492, 2012, 234, Adachi, et. al. J. Am. Chem. Soc, 134, 2012, 14706, Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013, 3766, Adachi, et. Al. J. Mater. Chem. C., 1, 2013, 4599, Adachi, et. al. J. Phys. Chem. A., 117, 2013, 5607. The entire contents of the above listed patent or literature documents are hereby incorporated by reference.

Some examples of suitable TADF light-emitting materials are listed in the following table:

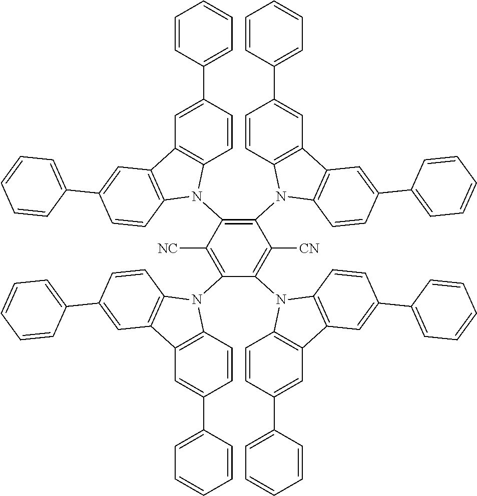 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21]

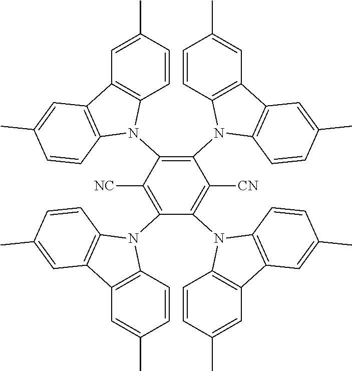

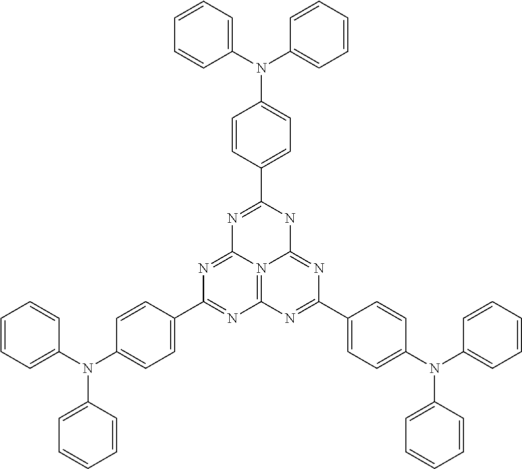

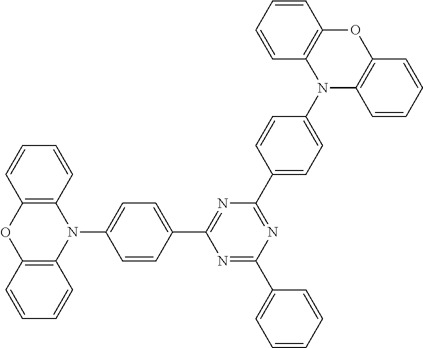 [SMILES: c1ccc(-c2nc(-c3ccc(N4c5ccccc5Oc5ccccc54)cc3)nc(-c3ccc(N4c5ccccc5Oc5ccccc54)cc3)n2)cc1]

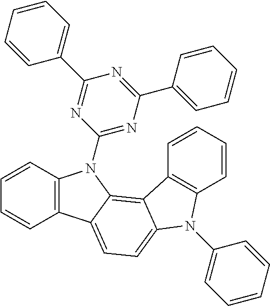 [SMILES: c1ccc(-c2nc(-c3ccccc3)nc(-n3c4ccccc4c4ccc5c(c6ccccc6n5-c5ccccc5)c43)n2)cc1]

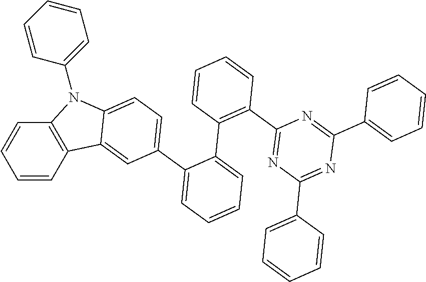 [SMILES: c1ccc(-c2nc(-c3ccccc3)nc(-c3ccccc3-c3ccccc3-c3ccc4c(c3)c3ccccc3n4-c3ccccc3)n2)cc1]

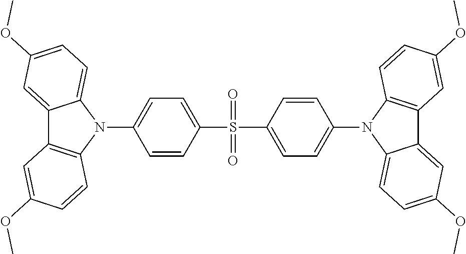 [SMILES: COc1ccc2c(c1)c1cc(OC)ccc1n2-c1ccc(S(=O)(=O)c2ccc(-n3c4ccc(OC)cc4c4cc(OC)ccc43)cc2)cc1]

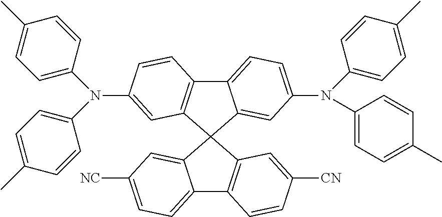

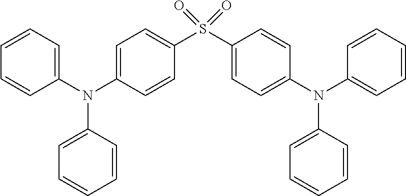 [SMILES: O=S(=O)(c1ccc(N(c2ccccc2)c2ccccc2)cc1)c1ccc(N(c2ccccc2)c2ccccc2)cc1]

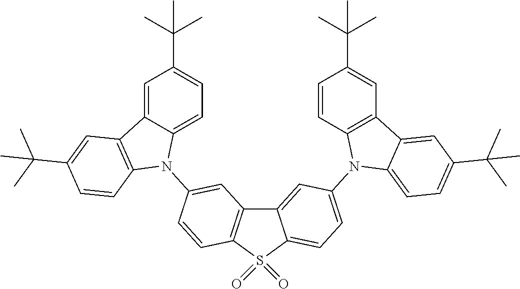 [SMILES: CC(C)(C)c1ccc2c(c1)c1cc(C(C)(C)C)ccc1n2-c1ccc2c(c1)-c1cc(-n3c4ccc(C(C)(C)C)cc4c4cc(C(C)(C)C)ccc43)ccc1S2(=O)=O]

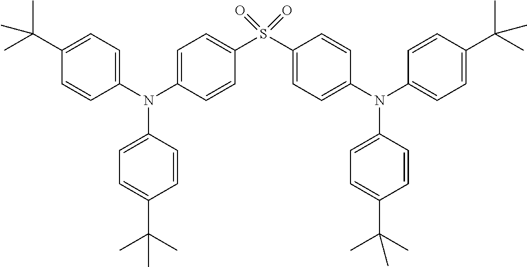 [SMILES: CC(C)(C)c1ccc(N(c2ccc(C(C)(C)C)cc2)c2ccc(S(=O)(=O)c3ccc(N(c4ccc(C(C)(C)C)cc4)c4ccc(C(C)(C)C)cc4)cc3)cc2)cc1]

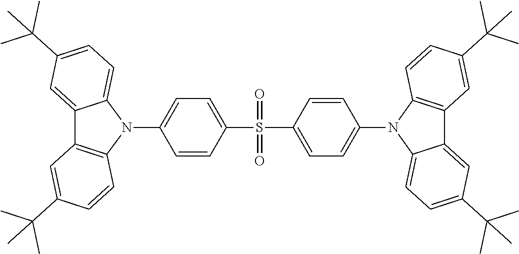 [SMILES: CC(C)(C)c1ccc2c(c1)c1cc(C(C)(C)C)ccc1n2-c1ccc(S(=O)(=O)c2ccc(-n3c4ccc(C(C)(C)C)cc4c4cc(C(C)(C)C)ccc43)cc2)cc1]

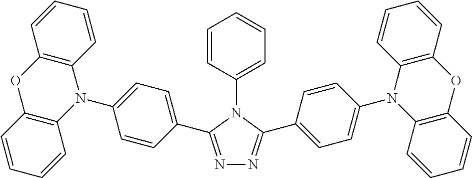 [SMILES: c1ccc(-n2c(-c3ccc(N4c5ccccc5Oc5ccccc54)cc3)nnc2-c2ccc(N3c4ccccc4Oc4ccccc43)cc2)cc1]

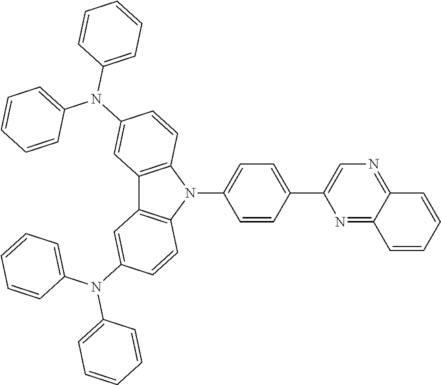 [SMILES: c1ccc(N(c2ccccc2)c2ccc3c(c2)c2cc(N(c4ccccc4)c4ccccc4)ccc2n3-c2ccc(-c3cnc4ccccc4n3)cc2)cc1]

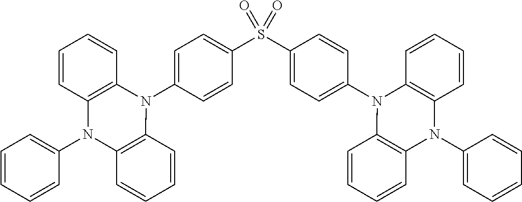 [SMILES: O=S(=O)(c1ccc(N2c3ccccc3N(c3ccccc3)c3ccccc32)cc1)c1ccc(N2c3ccccc3N(c3ccccc3)c3ccccc32)cc1]

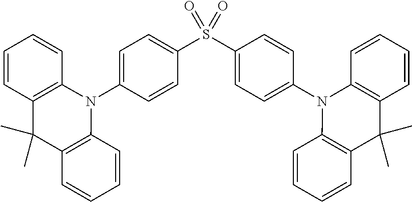

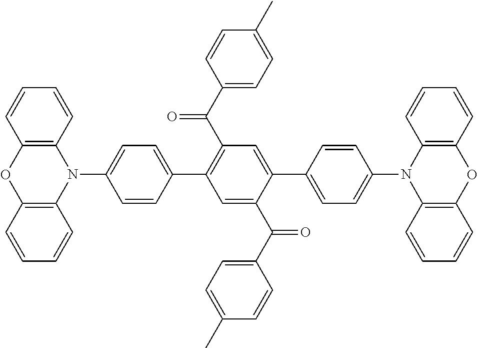 [SMILES: Cc1ccc(C(=O)c2cc(-c3ccc(N4c5ccccc5Oc5ccccc54)cc3)c(C(=O)c3ccc(C)cc3)cc2-c2ccc(N3c4ccccc4Oc4ccccc43)cc2)cc1]

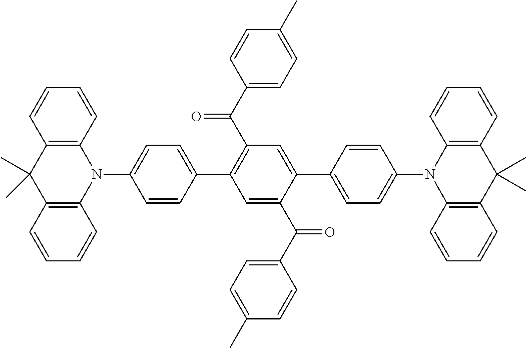

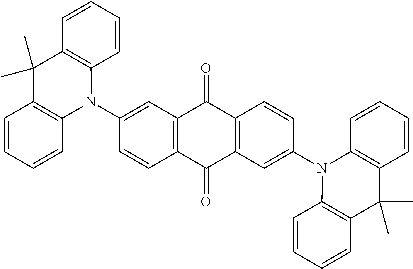

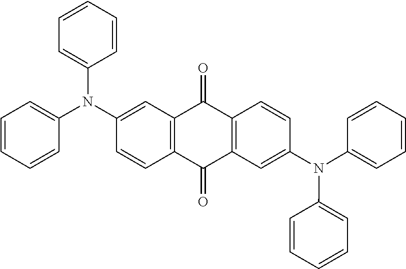 [SMILES: O=C1c2ccc(N(c3ccccc3)c3ccccc3)cc2C(=O)c2ccc(N(c3ccccc3)c3ccccc3)cc21]

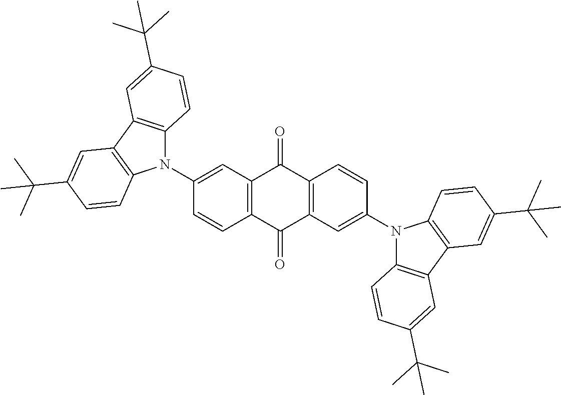 [SMILES: CC(C)(C)c1ccc2c(c1)c1cc(C(C)(C)C)ccc1n2-c1ccc2c(c1)C(=O)c1ccc(-n3c4ccc(C(C)(C)C)cc4c4cc(C(C)(C)C)ccc43)cc1C2=O]

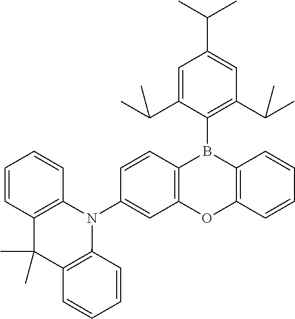 [SMILES: CC(C)c1cc(C(C)C)c(B2c3ccccc3Oc3cc(N4c5ccccc5C(C)(C)c5ccccc54)ccc32)c(C(C)C)c1]

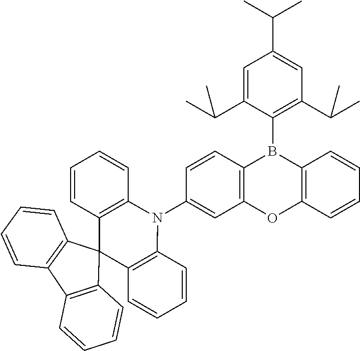 [SMILES: CC(C)c1cc(C(C)C)c(B2c3ccccc3Oc3cc(N4c5ccccc5C5(c6ccccc6-c6ccccc65)c5ccccc54)ccc32)c(C(C)C)c1]

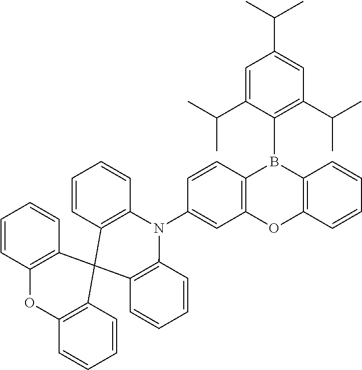 [SMILES: CC(C)c1cc(C(C)C)c(B2c3ccccc3Oc3cc(N4c5ccccc5C5(c6ccccc6Oc6ccccc65)c5ccccc54)ccc32)c(C(C)C)c1]

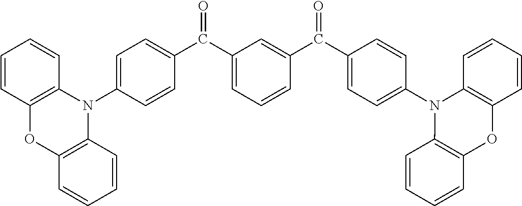 [SMILES: O=C(c1ccc(N2c3ccccc3Oc3ccccc32)cc1)c1cccc(C(=O)c2ccc(N3c4ccccc4Oc4ccccc43)cc2)c1]

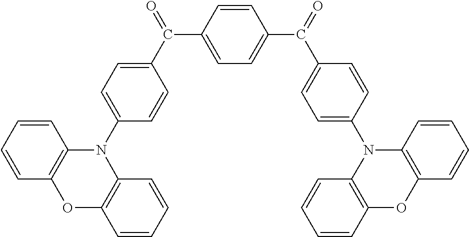 [SMILES: O=C(c1ccc(C(=O)c2ccc(N3c4ccccc4Oc4ccccc43)cc2)cc1)c1ccc(N2c3ccccc3Oc3ccccc32)cc1]

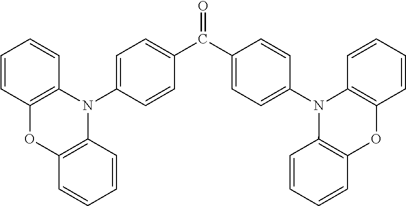 [SMILES: O=C(c1ccc(N2c3ccccc3Oc3ccccc32)cc1)c1ccc(N2c3ccccc3Oc3ccccc32)cc1]

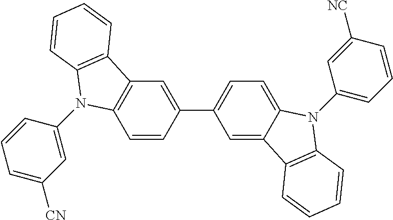 [SMILES: N#Cc1cccc(-n2c3ccccc3c3cc(-c4ccc5c(c4)c4ccccc4n5-c4cccc(C#N)c4)ccc32)c1]

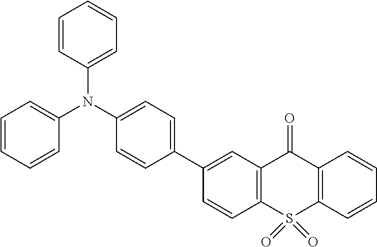 [SMILES: O=C1c2ccccc2S(=O)(=O)c2ccc(-c3ccc(N(c4ccccc4)c4ccccc4)cc3)cc21]

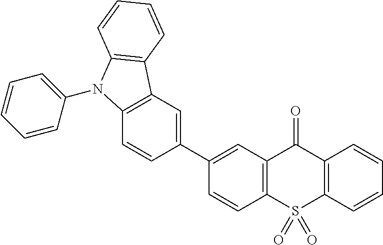 [SMILES: O=C1c2ccccc2S(=O)(=O)c2ccc(-c3ccc4c(c3)c3ccccc3n4-c3ccccc3)cc21]

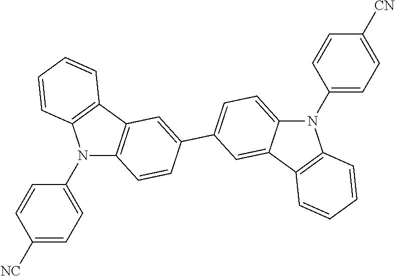 [SMILES: N#Cc1ccc(-n2c3ccccc3c3cc(-c4ccc5c(c4)c4ccccc4n5-c4ccc(C#N)cc4)ccc32)cc1]

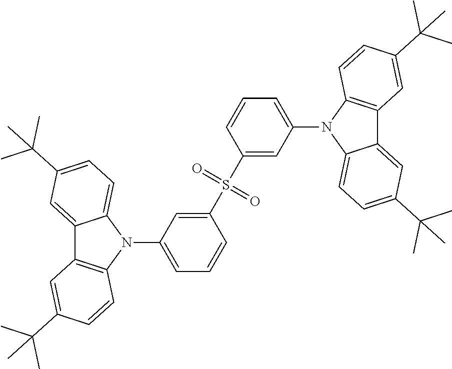 [SMILES: CC(C)(C)c1ccc2c(c1)c1cc(C(C)(C)C)ccc1n2-c1cccc(S(=O)(=O)c2cccc(-n3c4ccc(C(C)(C)C)cc4c4cc(C(C)(C)C)ccc43)c2)c1]

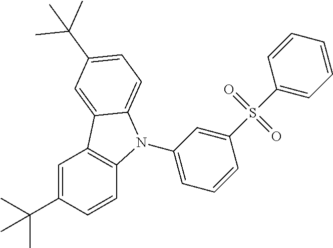 [SMILES: CC(C)(C)c1ccc2c(c1)c1cc(C(C)(C)C)ccc1n2-c1cccc(S(=O)(=O)c2ccccc2)c1]

3. Triplet Emitter

The triplet emitter is also called a phosphorescent emitter.

Preferably, the triplet emitter is a metal complex of the general formula M (L) n, wherein M is a metal atom; L is organic ligand, and is bonded or coordinated to M at one or more positions; n is a positive integer, preferably 1, 2, 3, 4, 5 or 6.

Preferably, these metal complexes is attached to a polymer by one or more positions, most preferably through an organic ligand.

Preferably, M is selected from transition metal elements or lanthanides or actinides.

More preferably, M is selected from Ir. Pt, Pd, Au, Rh, Ru, Os, Sm, Eu, Gd, Tb, Dy, Re, Cu or Ag.

Particularly preferably, M is selected from Os, Ir, Ru, Rh, Re, Pd, or Pt.

Preferably, the triplet emitter comprises a chelating ligand (i.e., a ligand), coordinated to the metal by at least two bonding sites.

More preferably, the triplet emitter comprises two or three identical or different bidentate or multidentate ligand. Chelating ligands help to improve stability of metal complexes.

The organic ligands may be selected from the group consisting of phenylpyridine derivatives, 7,8-benzoquinoline derivatives, 2 (2-thienyl) pyridine derivatives, 2 (1-naphthyl) pyridine derivatives, or 2 phenylquinoline derivatives. The organic ligands may be optionally substituted, for example, optionally substituted with fluoromethyl or trifluoromethyl.

The auxiliary ligand may be preferably selected from acetylacetonate or picric acid.

Preferably, the metal complex which may be used as the triplet emitter may have the following form:

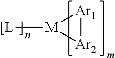

wherein M is a metal selected from transition metal elements, lanthanides or actinides;

Ar₁ may be the same or different cyclic group each time it is present, which comprises at least one donor atom, that is, an atom with a lone pair of electrons, such as nitrogen atom or phosphorus atom, which is coordinated to M through the donor atom;

Ar₂ may be the same or different cyclic group comprising at least one C atom and is coordinated to M through the C atom;

Ar₁ and Ar₂ are covalently bonded together, wherein each of them may carry one or more substituents which may also be joined together by substituents:

L may be the same or different at each occurrence and is an auxiliary ligand, preferably a bidentate chelating ligand, and most preferably a monoanionic bidentate chelating ligand;

m is 1, 2 or 3, preferably 2 or 3, and particularly preferably 3; and

n is 0, 1, or 2, preferably 0 or 1, particularly preferably 0.

Examples of triplet emitter materials and examples of applications thereof may be found in the following patent documents and references: WO 200070655, WO 200141512, WO 200202714, WO 200215645, EP 1191613, EP 1191612, EP 1191614, WO 2005033244, WO 2005019373, US 2005/0258742, WO 2009146770, WO 2010015307, WO 2010031485, WO 2010054731, WO 2010054728. WO 2010086089, WO 2010099852, WO 2010102709, US 20070087219 A1, US 20090061681 A1, US 20010053462 A1, Baldo, Thompson et al. Nature 403, (2000), 750-753, US 20090061681 A1, US 20090061681 A1, Adachi et al. Appl. Phys. Lett. 78 (2001), 1622-1624. J. Kido et al. Appl. Phys. Lett. 65 (1994), 2124, Kido et al. Chem. Lett. 657, 1990, US 2007/0252517 A1, Johnson et al., JACS 105, 1983, 1795, Wrighton, JACS 96, 1974, 998, Ma et al., Synth. Metals 94, 1998, 245, U.S. Pat. No. 6,824,895, U.S. Pat. No. 7,029,766, U.S. Pat. No. 6,835,469, U.S. Pat. No. 6,830,828, US 20010053462 A1, WO 2007095118 A1, US 2012004407A1, WO 2012007088A1, WO2012007087A1. WO 2012007086A1, US 2008027220A1, WO 2011157339A1, CN 102282150A and WO 2009118087A1. The entire contents of the above listed patent or literature documents are hereby incorporated by reference.

Examples of suitable triplet emitter are given in the following table:

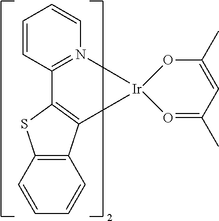

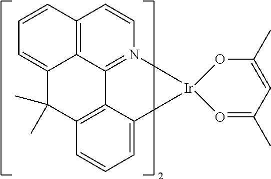

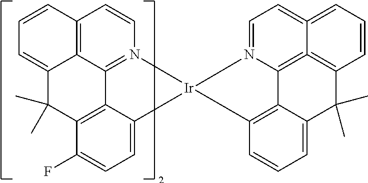

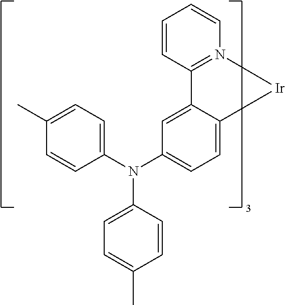

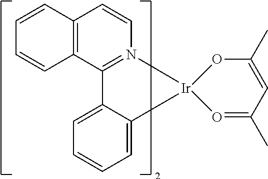

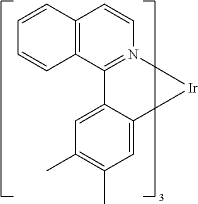

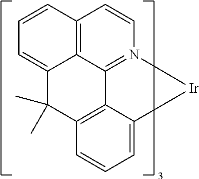

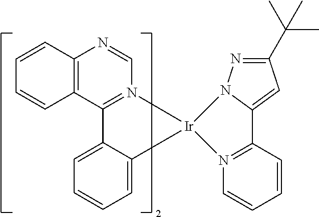

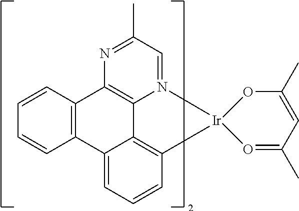

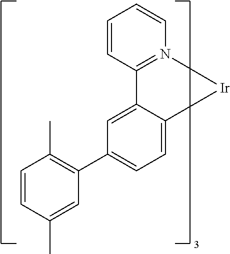

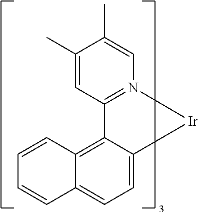

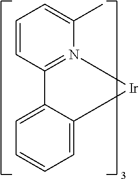

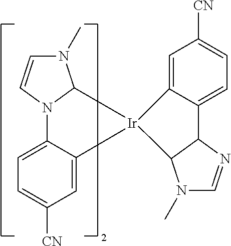

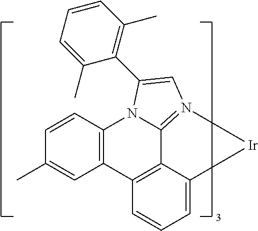

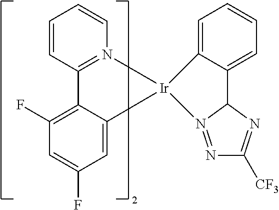

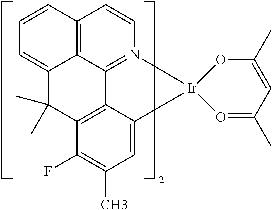

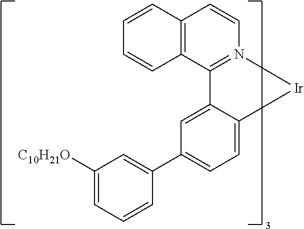

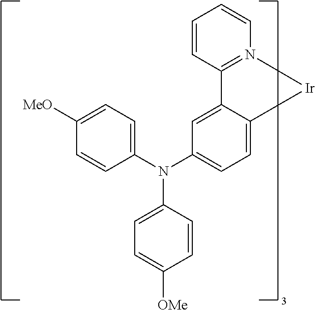

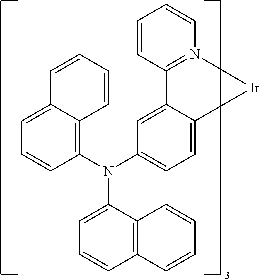

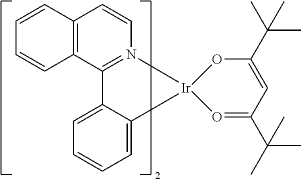

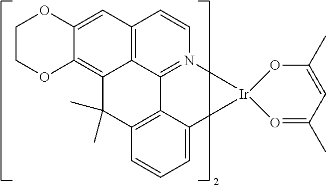

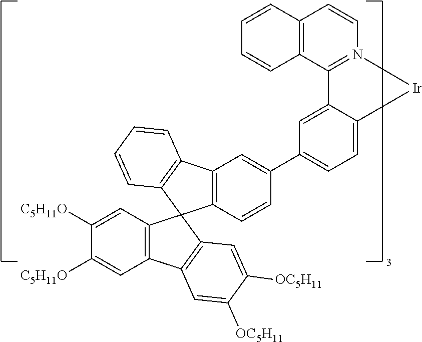

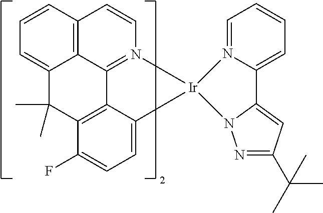

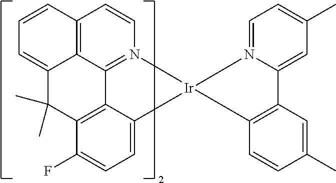

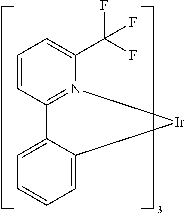

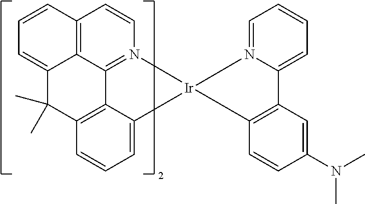

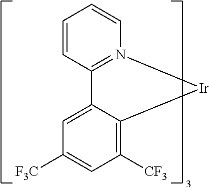

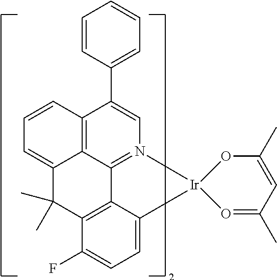

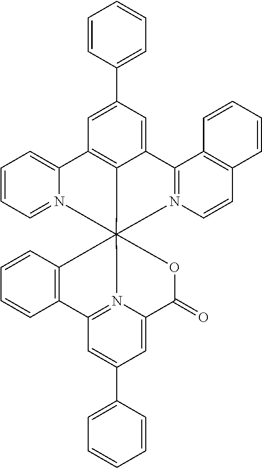

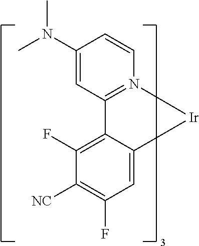

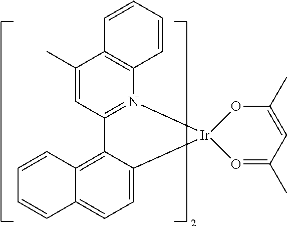

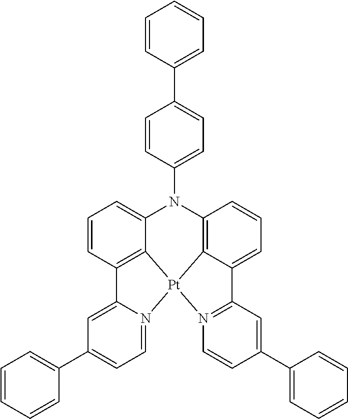

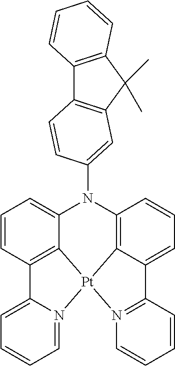

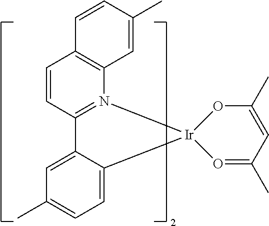

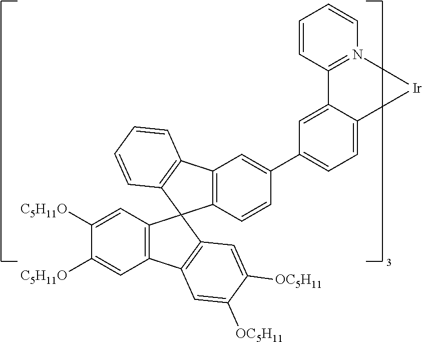

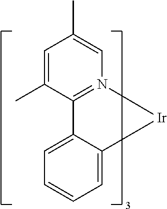

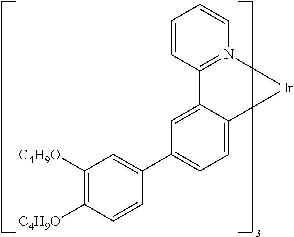

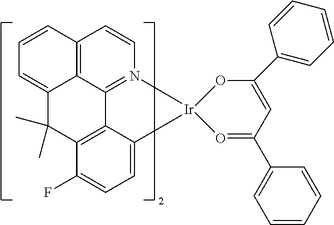

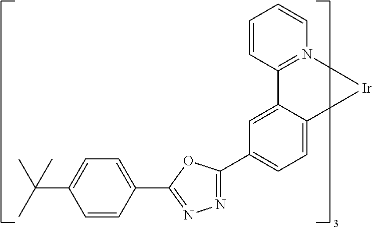

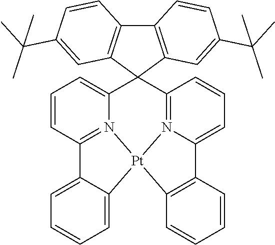

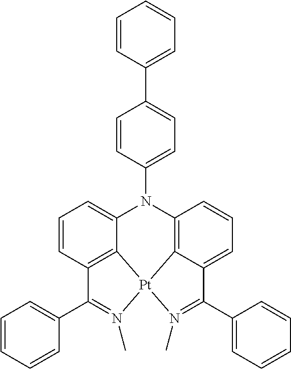

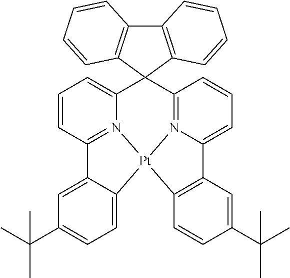

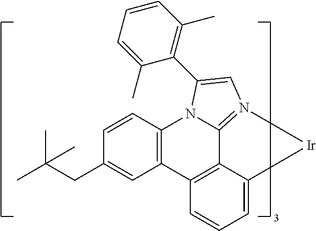

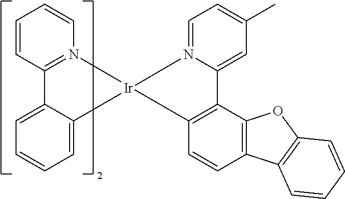

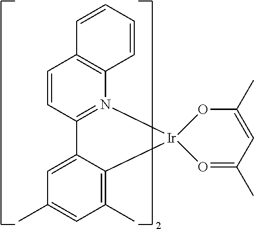

4. Light-Emitting Quantum Dot

In general, light-emitting quantum dots can emit light at a wavelength of 380 nanometers to 2500 nanometers. For example, it has been found that the quantum dots with a CdS core have an emission wavelength in the range of about 400 nm to 560 nm; the quantum dots with a CdSe core have an emission wavelength in the range of about 490 nm to 620 nm; the quantum dots with CdTe cores have an emission wavelength in the range of about 620 nanometers to 680 nanometers; the quantum dots with a InGaP core have an emission wavelength in the range of about 600 nanometers to 700 nanometers; the quantum dots with a PbS core have an emission wavelength in the range of about 800 nanometers to 2500 nanometers; the quantum dots with a PbSe core have an emission wavelength in the range of about 1200 nm to 2500 nm; the quantum dots with a CuInGaS core have an emission wavelength in the range of about 600 nm to 680 nm; the quantum dots with a ZnCuInGaS core have an emission wavelength in the range of about 500 nm to 620 nm; and the quantum dot with a CuInGaSe core have an emission wavelength in the range of about 700 nm to 1000 nm.

In a preferred embodiment, the quantum dot material includes one or more of an blue light quantum dot with a peak luminous wavelength of 450 nm to 460 nm, or green light quantum dot with a peak luminous wavelength of 520 nm to 540 nm, or red light quantum dot with a peak luminous wavelength of 615 nm to 630 nm, or their mixture.

Quantum dots may be selected for particular chemical compositions, topographical structures, and/or size dimensions to obtain light that emits a desired wavelength under electrical stimulation. The relationship between the luminescent properties of quantum dots and their chemical composition, morphology structure and/or size can be found in Annual Review of Material Sci., 2000, 30, 545-610; Optical Materials Express., 2012, 2, 594-628; and Nano Res, 2009, 2, 425-447. The entire contents of the above listed patent documents are hereby incorporated by reference.

The narrow particle size distribution of quantum dots enables them to have a narrower luminescence spectrum (J. Am. Chem. Soc., 1993, 115, 8706; and US 20150108405). In addition, depending on the various chemical composition and structure used, the size of the quantum dots needs to be adjusted within the above-mentioned size range to obtain the luminescent properties of the desired wavelength.

Preferably, the light-emitting quantum dots are semiconductor nanocrystals. In an embodiment, the size of the semiconductor nanocrystals is in the range of about 5 nanometers to about 15 nanometers. In addition, depending on the various chemical composition and structure used, the size of the quantum dots needs to be adjusted within the above-mentioned size range to obtain the luminescent properties of the desired wavelength.

The semiconductor nanocrystal includes at least one semiconductor material, wherein the semiconductor material may be selected from binary or polybasic semiconductor compounds of Group IV, II-VI, II-V, III-V, III-VI, IV-VI, I-III-VI, II-IV-VI, and II-IV-V of the periodic table, or their mixtures.

Examples of specific semiconductor materials include, but are not limited to: Group IV semiconductor compounds, including elemental Si, Ge and binary compounds SiC, SiGe; Group II-VI semiconductor compounds, including: binary compounds including CdSe, CdTe, CdO, CdS, CdSe, ZnS, ZnSe, ZnTe, ZnO, HgO, HgS, HgSe, and HgTe, temary compounds including CdSeS, CdSeTe, CdSTe, CdZnS, CdZnSe, CdZnTe, CgHgS, CdHgSe, ZnSeS, ZnSeTe, ZnSTe, HgSeS, HgSeTe, HgSTe, HgZnS, and HgSeSe, and quatemary compounds including CgHgSeS, CdHgSeTe, CgHgSTe, CdZnSeS, CdZnSeTe, HgZnSeTe, HgZnSTe, CdZnSTe, and HgZnSeS; Group III-V semiconductor compounds, including: binary compounds including ALN, AlP, AlAs, AlSb, GaN, GaP, GaAs, GaSb, InN, InP, InAs, and InSb, temary compounds including AlNP, AlNAs, AlNSb, AlPAs, AlPSb, GaNP, GaNAs, GaNSb, GaPAs, GaPSb, InNP, InNAs, InNSb, InPAs, and InPSb, and quaternary compounds include GaAlNAs, GaAlNSb, GaAlPAs, GaInNP, GaInNAs, GaInNSb, GaInPAs, GaInPSb, InAlNP, InAlNAs, InAlNSb, InAlPAs, and InAlPSb; Group IV-VI f semiconductor compounds, including: binary compounds including SnS, SnSe, SnTe, PbSe, PbS, and PbTe, temary compounds including SnSeS, SnScTe, SnSTe, SnPbS, SnPbSc, SnPbTe, PbSTe, PbSeS, and PbSeTe, and quaternary compounds including SnPbSSe, SnPbScTe, and SnPbSTe.

Preferably, the light-emitting quantum dot comprises a Group II-VI semiconductor compound, more preferably comprises CdSe. CdS, CdTe, ZnO, ZnSe, ZnS, ZnTe, HgS, HgSe, HgTe, CdZnSe and any combination thereof. In a suitable embodiment, this material is used as light-emitting quantum dots for visible light due to the relatively well-established synthesis scheme of CdSe and CdS.

Preferably, the light-emitting quantum dots comprise a Group III-V semiconductor compound, more preferably selected from InAs, InP, InN, GaN, InSb, InAsP, InGaAs, GaAs, GaP. GaSb, AlP, AlN, AlAs, AlSb, CdSeTe, ZnCdSe, and any combination thereof.

Preferably, the light-emitting quantum dots comprise Group IV-VI semiconductor compound, more preferably selected from the group consisting of PbSe, PbTe, PbS, PbSnTe, Tl₂SnTe₅, and any combination thereof.

Preferably, the quantum dots is in a core-shell structure. The core and the shell respectively include one or more identical or different semiconductor materials.

More preferably, In the quantum dots having a core-shell structure, the shell may comprise a monolayer or multilayer structure. The shell comprises one or more semiconductor materials that are the same as or different from the core. More preferably, the shell has a thickness of about 1 to 20 layers. Still more preferably, the shell has a thickness of about 5 to 10 layers. In certain embodiments, two or more shells grows on the surface of the quantum dot core.

Preferably, the semiconductor material used for the shell has a larger bandgap than the core. Particularly preferably, the core has a type I semiconductor heterojunction structure.

Preferably, the semiconductor material used for the shell has a smaller bandgap than the core.

Preferably, the semiconductor material used for the shell has the same or similar atomic crystal structure as the core. This choice is conducive to reducing the stress between the core and shell, making the quantum dots more stable.

Examples of suitable light-emitting quantum dots employing core-shell structures are (but not limited to):

Red light: CdSe/CdS. CdSe/CdS/ZnS, CdSe/CdZnS, and the like;

Green light: CdZnSe/CdZnS. CdSe/ZnS, and the like;

Blue light: CdS/CdZnS, CdZnS/ZnS, and the like.

Another object of the present disclosure is to provide material solutions for printed OLEDs.

Preferably, the teribenzocyclopentadiene compound disclosed herein has a molecular weight greater than or equal to 700 mol/kg, preferably greater than or equal to 900 mol/kg, more preferably greater than or equal to 900 mol/kg, still more preferably greater than or equal to 1000 mol/kg, and most preferably greater than or equal to 1100 mol/kg.

Preferably, the terbenzocyclopentadiene compound disclosed herein has a solubility at 25° C. in toluene greater than or equal to 10 mg/mL, preferably greater than or equal to 15 mg/mL, and most preferably greater than or equal to 20 mg/mL.

The present disclosure further relates to a formulation or ink comprising the above terbenzocyclopentadiene compound, the above polymer or the above mixture, and an organic solvent.

The present disclosure further provides a film comprising the compound or the polymer according to the present disclosure and prepared by a solution.

The viscosity and surface tension of ink are important parameters when the ink is used in the printing process. Appropriate surface tension parameter of the ink is suitable to the specific substrate and the specific printing method.

Preferably, the ink has a surface tension at working temperature or at 25° C. in the range of about 19 dyne/cm to about 50 dyne/cm, more preferably in the range of 22 dyne/cm to 35 dyne/cm, and most preferably in the range of 25 dyne/cm to 33 dyne/cm.

Preferably, the ink has a surface tension at the working temperature or at 25° C. in the range of about 1 cps to 100 cps, more preferably in the range of 1 cps to 50 cps, still more preferably in the range of 1.5 cps to 20 cps, and most preferably in the range of 4.0 cps to 20 cps. The formulation thus formulated will be suitable for inkjet printing.

The viscosity can be adjusted by various methods, such as by selecting the appropriate solvent and the concentration of the function material in the ink. The ink according to the present disclosure comprising the compound or polymer can facilitate the adjustment of the printing ink in an appropriate range according to the printing method used.

In general, the terbenzocyclopentadiene compound or the polymer in the formulation has a weight ratio in the range of 0.3 wt % to 30 wt %, preferably in the range of 0.5 wt % to 20 wt %, more preferably 0.5 wt/o to 15 wt %, still more preferably in the range of 0.5 wt % to 10 wt %, and most preferably in the range of 1 wt % to 5 wt %.

Preferably, the organic solvent is selected from solvents based on aromatics or heteroaromatics, especially aliphatic chain/ring substituted aromatic solvents, or aromatic ketone solvents, or aromatic ether solvents.

More preferably, the organic solvent is selected from the solvents based on aromatics or heteroaromatics, especially including p-diisopropylbenzene, pentylbenzene, tetrahydronaphthalene, cyclohexyl benzene, chloronaphthalene, 1,4-dimethylnaphthalene, 3-isopropylbiphenyl, p-cymene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, 1-methoxynaphthalene, cyclohexylbenzene, dimethylnaphthalene, 3-isopropylbiphenyl, p-cymene, 1-methylnaphthalene, 1,2,4-trichlorobenzene, 1,3-dipropoxybenzene, 4,4-difluorodiphenylmethane, 1,2-dimethoxy-4-(1-propenyl)benzene, diphenylmethane, 2-phenylpyridine, 3-phenylpyridine, N-methyldiphenylamine 4-isopropylbiphenyl, α,α-dichlorodiphenylmethane, 4-(3-phenylpropyl)pyridine, benzylbenzoate, 1,1-di(3,4-dimethylphenyl)ethane, 2-isopropylnaphthalene, dibenzylether, and the like; solvents based on ketones: 1-tetralone, 2-tetralone, 2-(phenylepoxy)tetralone, 6-(methoxyl)tetralone, acetophenone, phenylacetone, benzophenone, and derivatives thereof, such as 4-methylacetophenone, 3-methylacetophenone, 2-methylacetophenone, 4-methylphenylacetone, 3-methylphenylacetone, 2-methylphenylacetone, isophorone, 2,6,8-trimethyl-4-nonanone, fenchone, 2-nonanone, 3-nonanone, 5-nonanone, 2-demayone, 2,5-hexanedione, phorone, di-n-amyl ketone; aromatic ether solvents: 3-phenoxytoluene, butoxybenzene, benzylbutylbenzene, p-anisaldehyde dimethyl acetal, tetrahydro-2-phenoxy-2H-pyran, 1,2-dimethoxy 4-(1-propenyl)benzene, 1,4-benzodioxane, 1,3-dipropylbenzene, 2,5-dimethoxytoluene, 4-ethylphenetole, 1,2,4-trimethoxybenzene, 4-(1-propenyl)-1,2-dimethoxybenzene, 1,3-dimethoxybenzene, glycidyl phenyl ether, dibenzyl ether, 4-tert-butylanisole, trans-p-propenylanisole, 1,2-dimethoxybenzene 1-methoxynaphthalene, diphenyl ether, 2-phenoxymethyl ether, 2-phenoxytetrahydrofuran, ethyl-2-naphthyl ether, pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether; and ester solvents: alkyl octoate, alkyl sebacate, alkyl stearate, alkyl benzoate, alkyl phenylacetate, alkyl cinnamate, alkyl oxalate, alkyl maleate, alkyl lactone, alkyl oleate, and the like.

More preferably, the organic solvent is selected from aliphatic ketones, especially including 2-nonanone, 3-nonanone, 5-nonanone, 2-demayone, 2,5-hexanedione, 2,6,8-trimethyl-4-demayone, phorone, di-n-pentyl ketone, and the like, or aliphatic ethers, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethyl ether alcohol ethyl methyl ether, triethylene glycol butyl methyl ether, tripropylene glycol dimethyl ether, tetraethylene glycol dimethyl ether, and the like.

Preferably, the ink further includes another organic solvent. Examples of the another organic solvent include, but are not limited to, methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, decalin, indene, and/or mixtures thereof.

Preferably, the formulation is a solution.

Preferably, the formulation is a suspension.

The present disclosure further relates to the application of the formulation as the printing ink to make an organic electron device, preferably by a printing method or a coating method.

The appropriate printing technology or coating technology includes, but is not limited to inkjet printing, nozzle printing, typography, screen printing, dip coating, spin coating, blade coating, roller printing, twist roller printing, lithography, flexography, rotary printing, spray coating, brush coating or transfer printing, nozzle printing, slot die coating, and the like. The first preference is inkjet printing, slot die coating, nozzle printing, and typography. The solution or the suspension liquid may further includes one or more components, such as a surfactant compound, a lubricant, a wetting agent, a dispersant, a hydrophobic agent, a binder, to adjust the viscosity and the film forming property and to improve the adhesion property. The detailed information relevant to the printing technology and requirements of the printing technology to the solution, such as solvent, concentration, and viscosity, may be referred to Handbook of Print Media: Technologies and Production Methods, Helmut Kipphan, ISBN 3-540-67326-1.

Based on the above compound, the present disclosure further provides use of the above terbenzocyclopentadiene compound or the above polymer in an organic electronic device.

The organic electronic device may be selected from, but not limited to, an organic light-emitting diode (OLED), an organic photovoltaic (OPV), an organic light emitting electrochemical cell (OLEEC), an organic field effect transistor (OFET), an organic light emitting field effector, an organic laser, an organic spin electron device, an organic sensor, and an organic plasmon emitting diode, especially an OLED.

Preferably, the above terbenzocyclopentadiene compound or the above polymer is used in a light-emitting layer of the OLED device

The present disclosure further relates to an organic electronic device comprising the above terbenzocyclopentadiene compound or the above polymer;

In general, the organic electronic device includes a cathode, an anode, and a functional layer between the cathode and the anode, wherein the functional layer comprises the above terbenzocyclopentadiene compound or the above polymer.

The organic electronic device may be selected from, but not limited to, an organic light-emitting diode (OLED), an organic photovoltaic (OPV), an organic light emitting electrochemical cell (OLEEC), an organic field effect transistor (OFET), an organic light emitting field effector, an organic laser, an organic spin electron device, an organic sensor, and an organic plasmon emitting diode.

More preferably, the organic electronic device is an electroluminescence device, especially an OLED. The organic electronic device includes a substrate, an anode, a light-emitting layer, and a cathode. The organic electronic device may optionally include a hole transport layer and/or an electron transport layer.

Preferably, the hole transport layer comprises the above terbenzocyclopentadiene compound or the above polymer.

Preferably, the electron transport layer comprises the above terbenzocyclopentadiene compound or the above polymer.

Preferably; the light-emitting layer comprises the above terbenzocyclopentadiene compound or the above polymer.

More preferably, the light-emitting layer comprises the above terbenzocyclopentadiene compound or the above polymer, and a light-emitting material. The light-emitting material may be selected from a fluorescent light emitter, a phosphorescent light emitter, a TADF material or a light-emitting quantum dot.

The structure of the electroluminescence device is briefly described below, but it is not limited thereto.

The substrate may be opaque or transparent. The transparent substrate may be used to make the transparent luminescent device, which may be referred to, for example, Bulovic et al., Nature, 1996, 380, page 29 and Gu et al., Appl. Phys. Lett., 1996, 68, page 2606. The substrate may be rigid or elastic. The substrate may be plastic, metal, a semiconductor wafer, or glass. Preferably, the substrate has a smooth surface. The substrate without any surface defects is the particular ideal selection. In one preferred embodiment, the substrate is flexible and may be selected from a polymer thin film or a plastic which have the glass transition temperature Tg larger than 150° C., preferably larger than 200° C., more preferably larger than 250° C., most preferably larger than 300° C. Suitable examples of the flexible substrate are polyethylene terephthalate (PET) and polyethylene 2,6-naphthalate (PEN).

The anode may include a conductive metal, metallic oxide, or a conductive polymer. The anode can inject holes easily into the hole injection layer (HIL), the hole transport layer (HTL), or the light-emitting layer. In one embodiment, the absolute value of the difference between the work function of the anode and the HOMO energy level or the valence band energy level of the emitter in the light-emitting layer or of the p-type semiconductor material of the HIL or HTL or the electron blocking layer (EBL) is smaller than 0.5 eV, preferably smaller than 0.3 cV, most preferably smaller than 0.2 eV. Examples of the anode material include, but are not limited to Al, Cu, Au, Ag, Mg, Fe, Co, Ni, Mn, Pd, Pt, ITO, aluminum-doped zinc oxide (AZO), and the like. Other suitable anode materials are known and may be easily selected by one of ordinary skilled in the art. The anode material may be deposited by any suitable technologies, such as the suitable physical vapor deposition method which includes a radio frequency magnetron sputtering, a vacuum thermal evaporation, an electron beam, and the like. In some embodiments, the anode is patterned and structured. A patterned ITO conductive substrate may be purchased from market to prepare the device according to the present disclosure.

The cathode may include a conductive metal or metal oxide. The cathode can inject electrons easily into the electron injection layer (EIL) or the electron transport layer (ETL), or directly injected into the light-emitting layer. In one embodiment, the absolute value of the difference between the work function of the cathode and the LUMO energy level or the valence band energy level of the emitter in the light-emitting layer or of the n type semiconductor material as the electron injection layer (EIL) or the electron transport layer (ETL) or the hole blocking layer (HBL) is smaller than 0.5 eV, preferably smaller than 0.3 eV, most preferably smaller than 0.2 eV. In principle, all materials capable of using as the cathode of the OLED may be used as the cathode material of the device of the present disclosure. Examples of the cathode material include, but are not limited to, Al, Au, Ag, Ca, Ba, Mg, LiF/Al, MgAg alloy, BaF₂/Al, Cu, Fe, Co, Ni, Mn, Pd, Pt, ITO, and the like. The cathode material may be deposited by any suitable technologies, such as the suitable physical vapor deposition method which includes a radio frequency magnetron sputtering, a vacuum thermal evaporation, an electron beam, and the like.

The OLED may further comprise other functional layers such as hole injection layer (HIL), hole transport layer (HTL), electron blocking layer (EBL), electron injection layer (EIL), electron transport layer (ETL), and hole blocking layer (HBL), or a combination thereof. Materials suitable for use in these functional layers are described in detail above.

In a preferred embodiment, the light-emitting layer of the electroluminescence device comprises the above terbenzocyclopentadiene compound or the above polymer, and is prepared by a method of solution processing.

The electroluminescence device has a light emission wavelength between 300 and 1000 nm, more preferably between 350 and 900 nm, and more preferably between 400 and 800 nm.

The present disclosure further relates to the use of the above organic electronic device in various electronic devices, including, but not limited to display devices, lighting devices, light sources, sensors, and the like.

The present invention will be described below with reference to following preferred embodiments, but is not limited thereto. It should be understood that the scope of the present invention is defined by the appended claims. Those skilled in the art will appreciate that, guided by the concept of the present invention, various modifications can be made to the embodiments of the invention, without departing from the spirit and scope of the invention as claimed. The method for synthesizing the compound of the present disclosure is exemplified below, but the present disclosure is not limited to the following examples.

### Example 1. Synthesis of Compound (2-2)

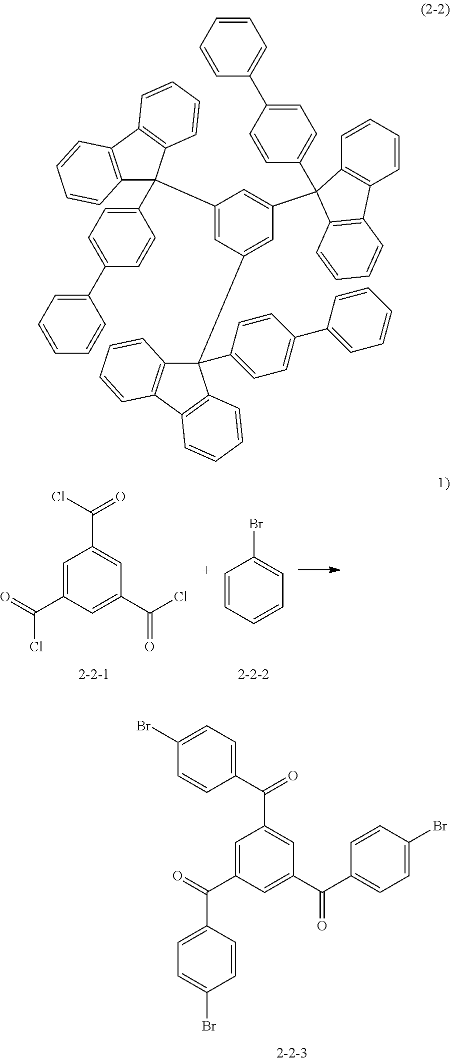

To a 500 mL two-necked flask 1,3,5-benzenetricarbonyl trichloride (26.5 g, 100 mmol) and bromobenzene (62.8 g, 400 mmol) were added and anhydrous aluminium chloride (66.5 g, 500 mmol) was added in batches t under stirring. After addition, the reaction solution was stirred and reacted at room temperature for 0.5 hours, heated at 90° C. for two hours before the end of reaction. The reaction product was slowly added to a hydrochloride aqueous solution, and suction-filtered, and the residue was recrystallized from a mixed solution of dichloromethane/ethanol, with a yield rate of 90%.

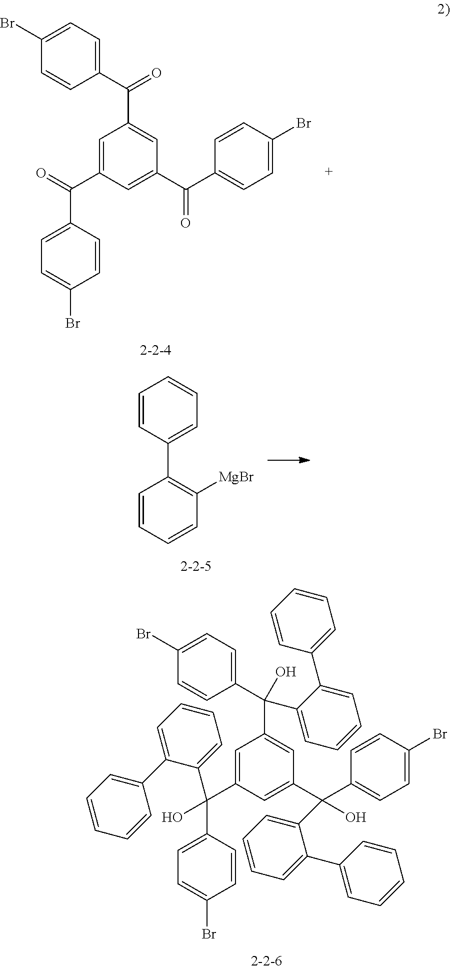

To a THF solution of compound 2-2-4 (18.8 g, 20 mmol) the pre-prepared solution (45 mmol) of 2-biphenyl magnesium bromide in tetrahydrofuran was added. The reaction solution was heated to 60° C. and reacted for 12 hours. Then 60 mL of deionized water was added slowly and reacted for 0.5 hours while holding temperature before the end of reaction. Most THF in the reaction solution was removed by rotatory evaporation, and the reaction solution was extracted with dichloromethane, and washed once with hydrochloride aqueous solution and twice with water. The organic phase was dried over anhydrous magnesium sulfate, spin-dried, and used for the next reaction without further purification.

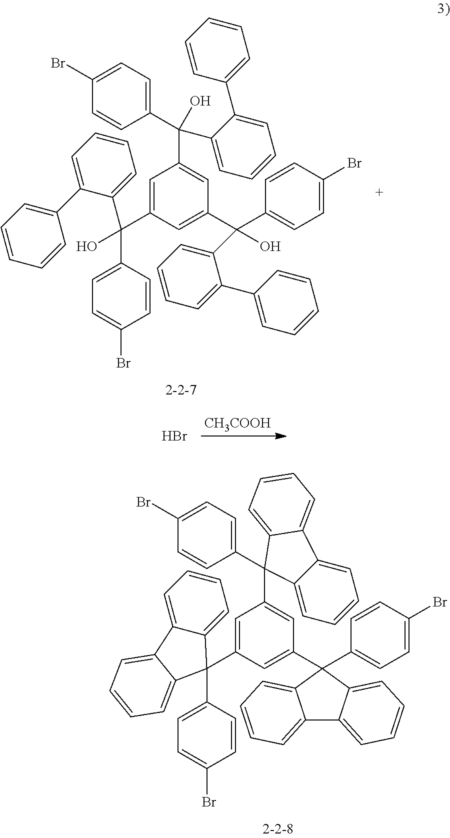

To a 150 mL one-necked flask compound 2-2-7 (27.2 g, 25 mmol) resulted from the previous step, 20 mL of hydrobromic acid and 40 mL of acetic acid were added. The reaction solution was stirred, heated and reacted at 120° C. for two hours before the end of reaction. A solid was precipitated and the supernatant in the reaction solution was discarded. To the solid methanol was added, and the reaction mixture was suction-filtered. The residue was recrystallized from a mixed solution of dichloromethane/ethanol, with a yield rate of 90%.

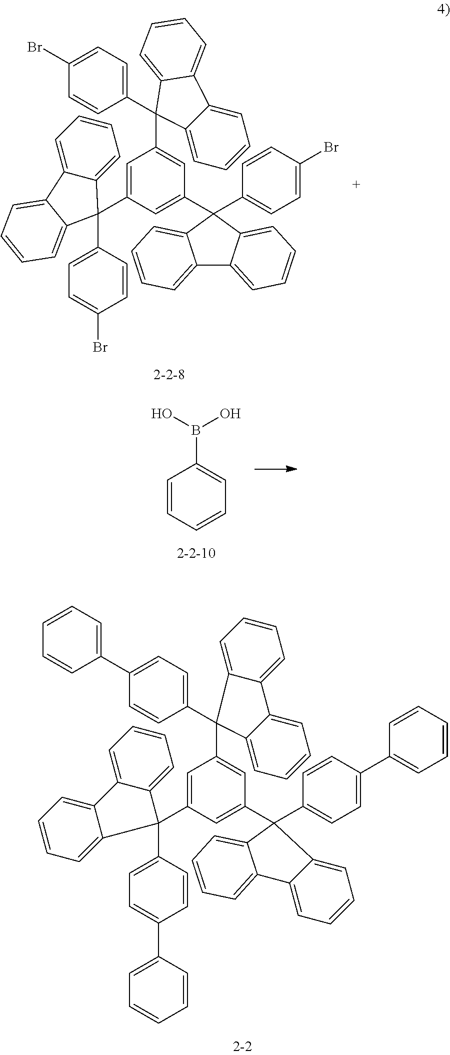

To a 150 mL two-necked flask compound 2-2-9 (15.5 g, 15 mmol), phenylboronic acid (7.3 g, 60 mmol), sodium carbonate (15.9 g, 150 mmol), tetrabutylammonium bromide (0.48 g, 1.5 mmol), tetrakis(triphenylphosphine)palladium (0.52 g, 0.45 mmol), 60 mL of 1,4-dioxane and 10 mL of water were added. The reaction solution was heated at 90° C., stirred and reacted for 12 hours before the end of the react. The reaction solution was added to 400 mL of water and suction-filtered. The residue was recrystallized from a mixed solution of dichloromethane/petroleum ether, with a yield rate of 90%.

### Example 2. Synthesis of Compound (2-6)

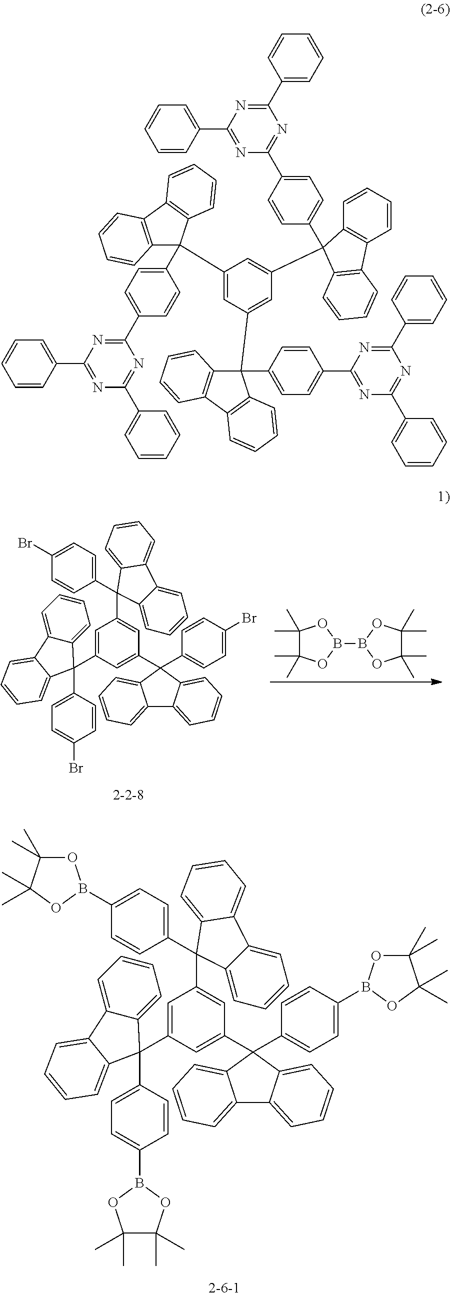

To a 300 mL two-necked flask compound (2-2-8) (12.78 g, 30 mmol), bis(pinacolato)diboron (25.4 g, 100 mmol), Pd(dppf)Cl, (1.5 mmol), potassium acetate (100 mmol) and 150 mL of 1,4-dioxane were added. The reaction solution was heated at 110° C., stirred and reacted for 12 hours before the end of reaction. The reaction solution was added to 500 mL of water and suction-filtered. The residue was collected and purified with silica gel, with a yield rate of 80%.

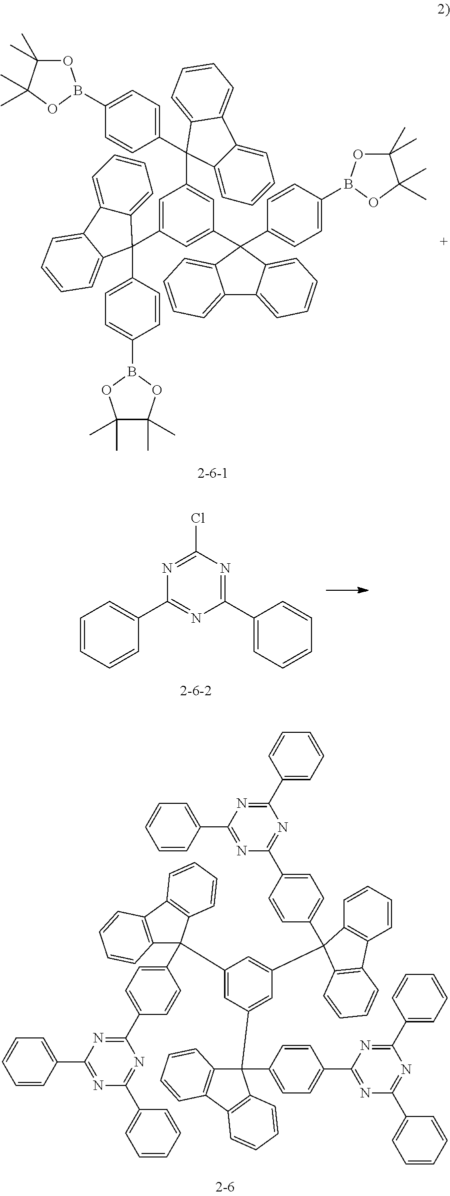

To a 250 mL two-necked flask compound (2-6-1) (11.77 g, 10 mmol), compound (2-6-2) (9.38 g, 35 mmol), sodium carbonate (4.24 g, 40 mmol), tetrabutylammonium bromide (1.6 g, 5 mmol), tetrakis(triphenylphosphine) palladium (0.52 g, 0.45 mmol), 100 mL 1,4-dioxane and 20 mL of water were added under the nitrogen atmosphere. The reaction solution was heated at 90° C., stirred and reacted for 12 hours before the end of reaction. The reaction solution was added to 400 mL of water, extracted with dichloromethane, and washed with water for three times. The organic solution was collected and purified with silica gel, with a yield rate of 85%.

### Example 3. Energy Structure of the Organic Compound

The energy level of the organic material can be calculated by quantum computation, for example, using TD-DFT (time-dependent density functional theory) by Gaussian03W (Gaussian Inc.), the specific simulation methods of which can be found in WO2011141110 Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-density functional theory) “TD-SCF/DFT/Default SpiniB3PW91” and the basis set “6-31G (d)” (Charge 0/Spin Singlet). The HOMO and LUMO levels are calculated using the following calibration formula, wherein S₁ and T₁ are used directly.

HOMO(eV)=((HOMO(G)×27.212)−0.9899)/1.1206

LUMO(eV)=((LUMO(G)×27.212)−2.0041)/1.385

wherein, HOMO(G) and LUMO(G) are the direct calculation results of Gaussian 03W, in units of Hartree. The results are shown in Table 1:

TABLE 1

| | HOMO | LUMO | T1 | S1 |
|---|---|---|---|---|
| Material | [eV] | [eV] | [eV] | [eV] |
| 2-2 | −6.03 | −2.18 | 2.95 | 3.18 |
| 2-6 | −6.19 | −2.84 | 3.02 | 3.07 |
| B3PYMPM | −5.33 | −2.20 | 2.72 | 3.28 |

### Example 4. Preparation and Characterization of a Solution-Processing OLED Device

The structure of a solution-processing OLED device is shown as follow:

ITO/PEDOT(80 nm)/TFB(20 nm)/Host material:
Emitter(15 wt %)(45 nm)/B3PYMPM(35)/LiF(1 nm)/Al(100 nm). Wherein the soluble Emitter is shown in the following formula,

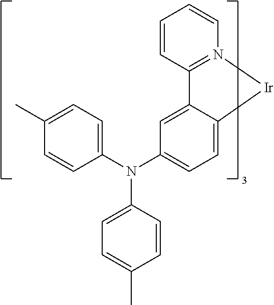

The hole transport material (i.e. TFB) (H.W. Sands Corp.) is shown as follow:

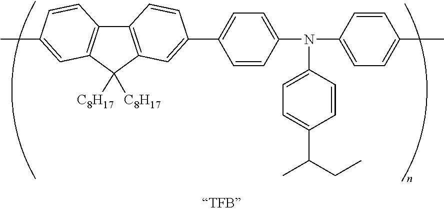

PEDOT, TFB and light-emitting layer were all formed by spin coating. In the hole transport layer TFB, a solution of TFB in toluene with a solubility of 6 mg/Ml was used. In the light-emitting layer, a mixture was used. The host material: Emitter (15 wt %) in toluene with a solubility of 20 mg/mL. B3PYMPM (40 nm), LiF (1 nm), and Al (100 nm) were subjected to a thermal evaporation deposition in high vacuum (1×10⁻⁶ mbar); finally the device was packaged by UV curing resin in the nitrogen glove box.

TABLE 2

| OLED Device | Host Material | Maximum External Quantum Efficiency % |
|---|---|---|
| OLED1 | (2-2) | 15.2% |
| OLED2 | (2-6) | 13.7% |
| OLED3 | Ref1 | 6% |

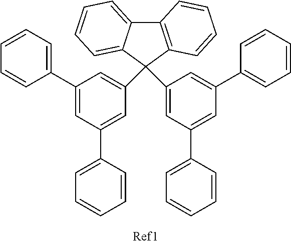 [SMILES: c1ccc(-c2cc(-c3ccccc3)cc(C3(c4cc(-c5ccccc5)cc(-c5ccccc5)c4)c4ccccc4-c4ccccc43)c2)cc1]

The commonly used evaporated host material CBP cannot be made into OLED devices due to its poor solubility in common solvents such as toluene. The host material Ref1 is dissolved in toluene, but may have poor film-forming property due to a very small molecular weight. However, the host materials (2-2) and (2-6) of the present disclosure have good solubility in toluene and very good film-forming property.

The current-voltage (J-V) characteristics of each OLED device are characterized by characterization equipment, while important parameters such as efficiency, lifetime and external quantum efficiency were recorded. As shown in Table 2, the luminous efficiency of OLED1 and OLED2 is much higher than that of OLED. Meanwhile, the lifetime of OLED1 and OLED2 is 30 times and 25 times or above of that of OELD3, respectively. It can be seen that the OLED devices prepared by using the organic compound of the present disclosure as a soluble host has greatly improved its efficiency and lifetime.

What described above are several embodiments of the present disclosure, and they are specific and in details, but not intended to limit the scope of the present disclosure. It will be understood by those skilled in the art that various modifications and improvements can be made without departing from the concept of the present disclosure, and all these modifications and improvements are within the scope of the present disclosure. The scope of the present disclosure shall be subject to the appended claims.

What is claimed is:

1. A terbenzocyclopentadiene compound, which has the following general formula (1): 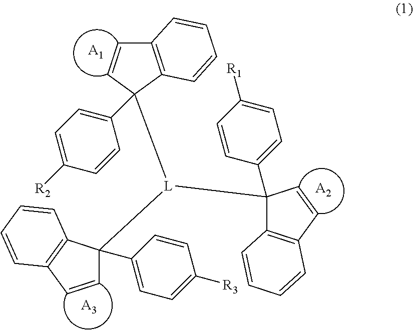
wherein L is a linking unit, and selected from an aryl group containing 6 to 40 carbon atoms or a heteroaryl group containing 3 to 40 carbon atoms;
A₁, A₂, or A₃ is selected from an aryl group containing 6 to 30 carbon atoms or a heteroaryl group containing 3 to 30 carbon atoms;
R₁, R₂, or R₃ is selected from H, D, F, CN, an alkyl group containing 1 to 30 carbon atoms, a cycloalkyl group containing 3 to 30 carbon atoms, an aromatic hydrocarbon group containing 6 to 60 carbon atoms, and an aromatic heterocyclic group containing 3 to 60 atoms, and one or more positions of R₁, R₂, or R₃ may be substituted by H, D, F, CN, alkyl, aralkyl, alkenyl, alkynyl, nitrile group, amino, nitro, acyl, alkoxy, carbonyl, sulfonyl group, cycloalkyl, or hydroxy.

2. The terbenzocyclopentadiene compound according to claim 1, wherein the terbenzocyclopentadiene compound has a glass transition temperature Tg greater than or equal to 100° C.

3. The terbenzocyclopentadiene compound according to claim 1, wherein L is selected from benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, or spirosilafluorene.

4. The terbenzocyclopentadiene compound according to claim 1, wherein L is any one selected from the following structural units or the units formed by substituting any one of the following structural units:

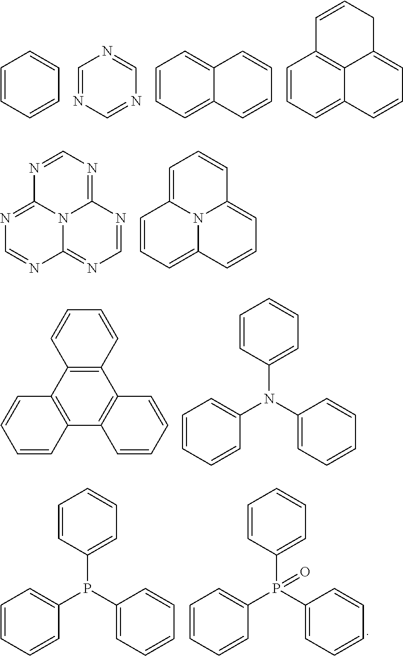

5. The terbenzocyclopentadiene compound according to claim 1, wherein A₁, A₂, or A₃ is one selected from the following structural groups: 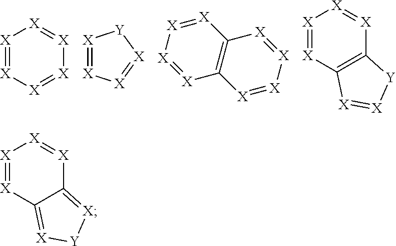
wherein X is selected from CR¹ or N;
Y is selected from CR²R³, SiR²R³, NR², C(═O), S or O;
R¹, R², or R³ is one or a combination of more than one selected from H, D, a linear alkyl group containing 1 to 20 C atoms, an alkoxy group containing 1 to 20 C atoms, a thioalkoxy group containing 1 to 20 C atoms, a branched alkyl group containing 3 to 20 C atoms, a cyclic alkyl group containing 3 to 20 C atoms, an alkoxy or thioalkoxy group containing 3 to 20 C atoms, a silyl group containing 3 to 20 C atoms, a substituted keto group containing 1 to 20 C atoms, a alkoxycarbonyl group containing 2 to 20 C atoms, an aryloxycarbonyl group containing 7 to 20 C atom, a cyano group (—CN), a carbamoyl group (—C(═O)NH₂), a haloformyl group, a formyl group (—C(═O)—H), an isocyano group, an isocyanate group, a thiocyanate group, an isothiocyanate group, a hydroxyl group, a nitryl group, a CF₃ group, Cl, Br, F, an crosslinkable group, an aromatic ring system containing 5 to 40 ring atoms, a heteroaromatic ring system containing 5 to 40 ring atoms, a substituted aromatic ring system containing 5 to 40 ring atoms, a substituted heteroaromatic ring system containing 5 to 40 ring atoms, an aryloxy group containing 5 to 40 ring atoms, and a heteroaryloxy group containing 5 to 40 ring atoms.

6. The terbenzocyclopentadiene compound according to claim 1, wherein A₁, A₂, or A₃ is one selected from the following structural groups or substituted groups formed by one of the following structural groups being further substituted: 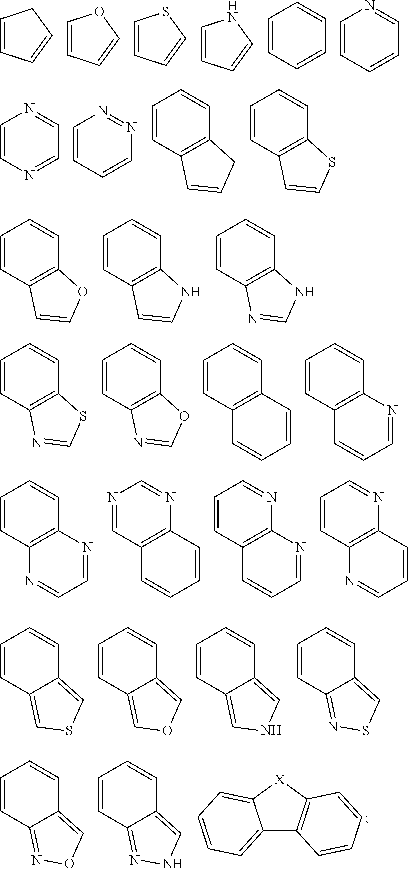
wherein X is selected from the group consisting of N(R), B(R), C(R)₂, Si(R)₂, O, S, C═N(R), C═C(R)₂, P(R), P(═O)R, S═O, and SO₂, or X is absent; X is preferably N(R), C(R)₂, O, or S;
R is selected from an alkyl group containing 1 to 30 carbon atoms, a cycloalkyl group containing 3 to 30 carbon atoms, an aromatic hydrocarbon group containing 6 to 60 carbon atoms, or an aromatic heterocyclic group containing 3 to 60 carbon atoms, and one or more positions of R may be substituted by H, D, F, CN, alkyl, aralkyl, alkenyl, alkynyl, nitrile group, amino, nitro, acyl, alkoxy, carbonyl, sulfonyl group, cycloalkyl, or hydroxy.

7. The terbenzocyclopentadiene compound according to claim 1, wherein A₁, A2, or A₃ is one selected from the following structural groups or substituted groups formed by one of the following structural groups being further substituted:

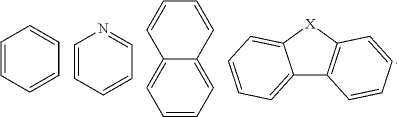

8. The terbenzocyclopentadiene compound according to claim 1, wherein R₁, R₂ or R₃ is selected from methyl, benzene, naphthalene, anthracene, phenanthrene, pyrene, pyridine, pyrimidine, triazine, fluorene, dibenzothiophene, silafluorene, carbazole, thiophene, furan, thiazole, triphenylamine, triphenylphosphanoxid, tetraphenylsilane, spirofluorene, or spirosilafluorene.

9. The terbenzocyclopentadiene compound according to claim 1, wherein the terbenzocyclopentadiene compound is one selected from compounds having the following structural formula: 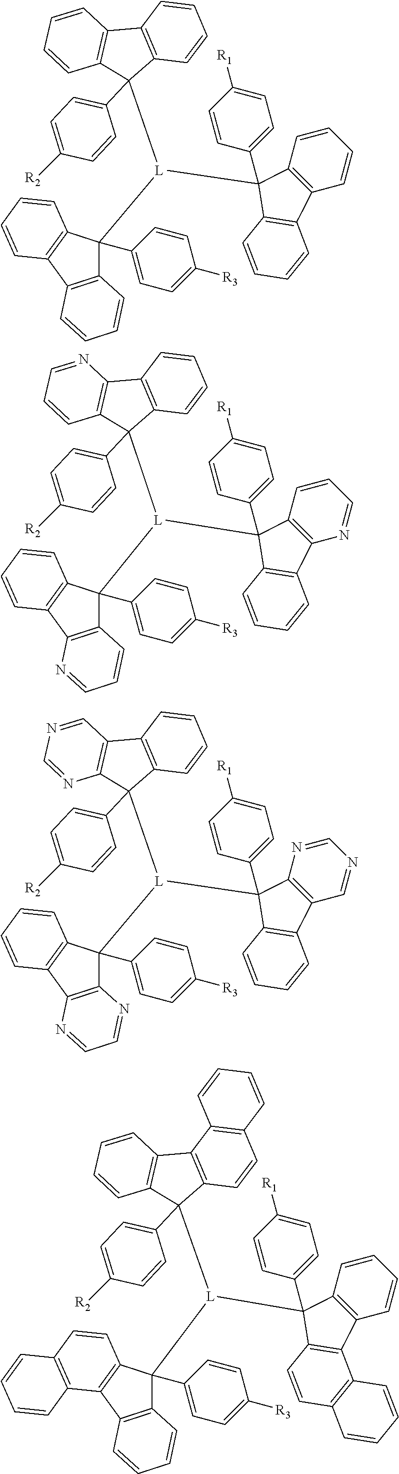 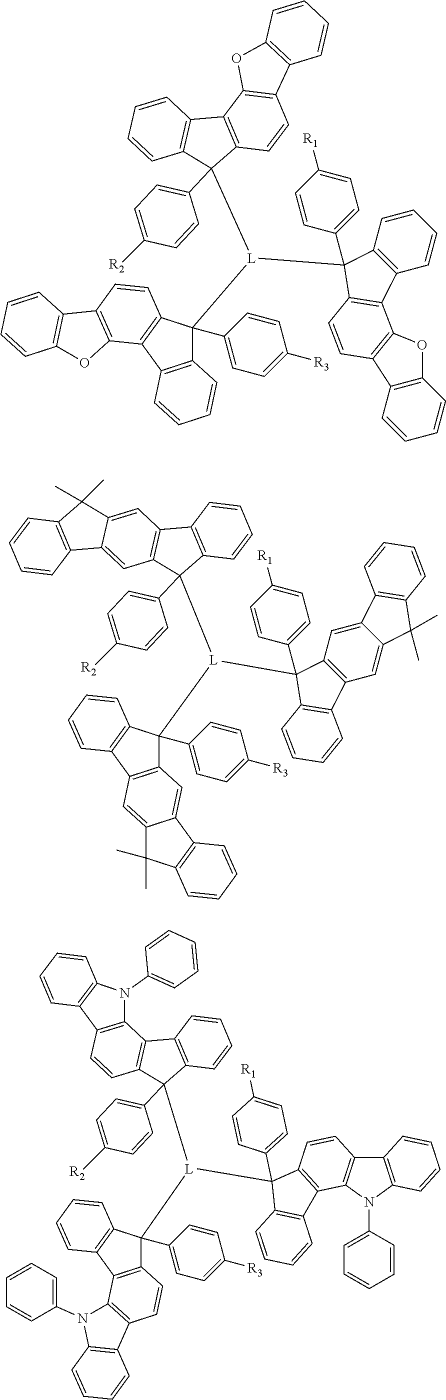 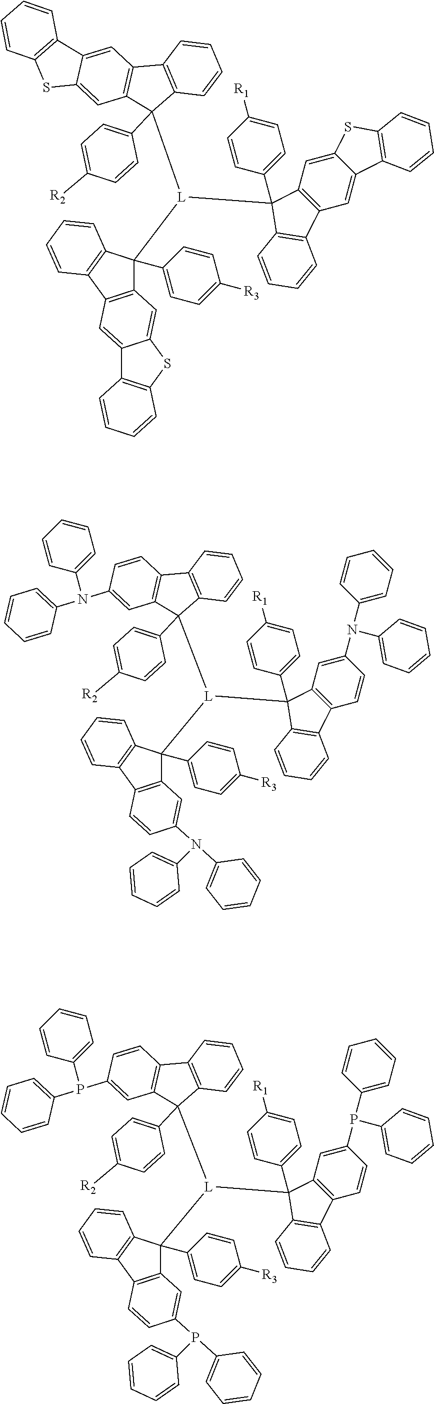 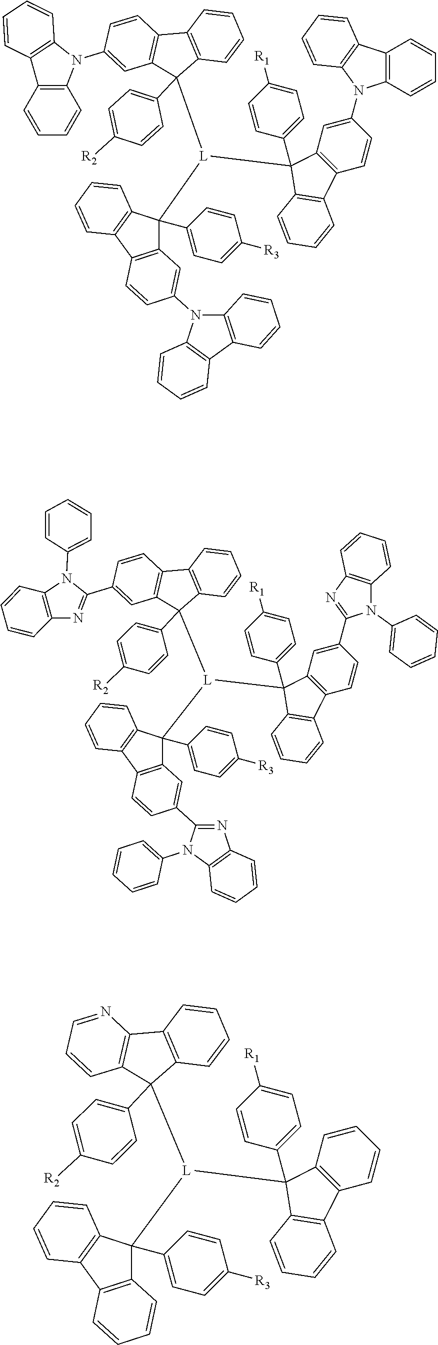 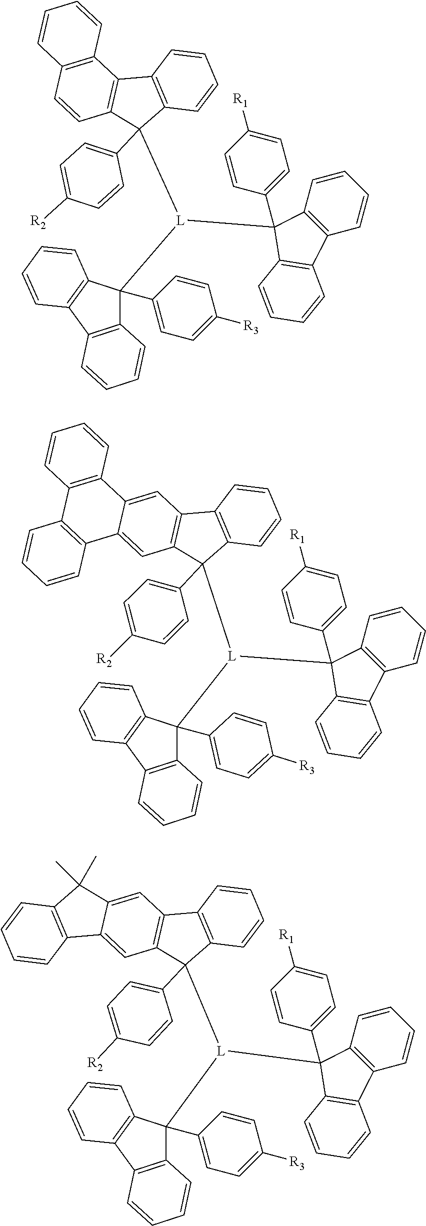 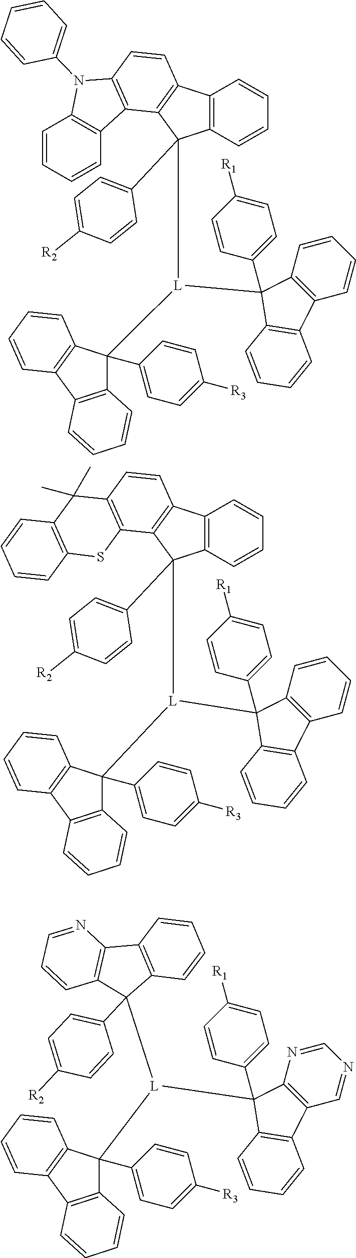 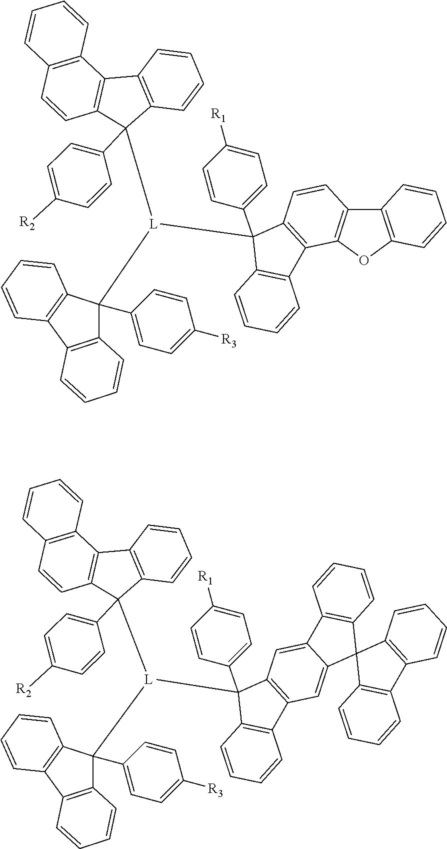
wherein L, R₁, R₂ and R₃ are defined as above

10. The terbenzocyclopentadiene compound according to claim 1, wherein the terbenzocyclopentadiene compound is one selected from compounds having the following structural formula: 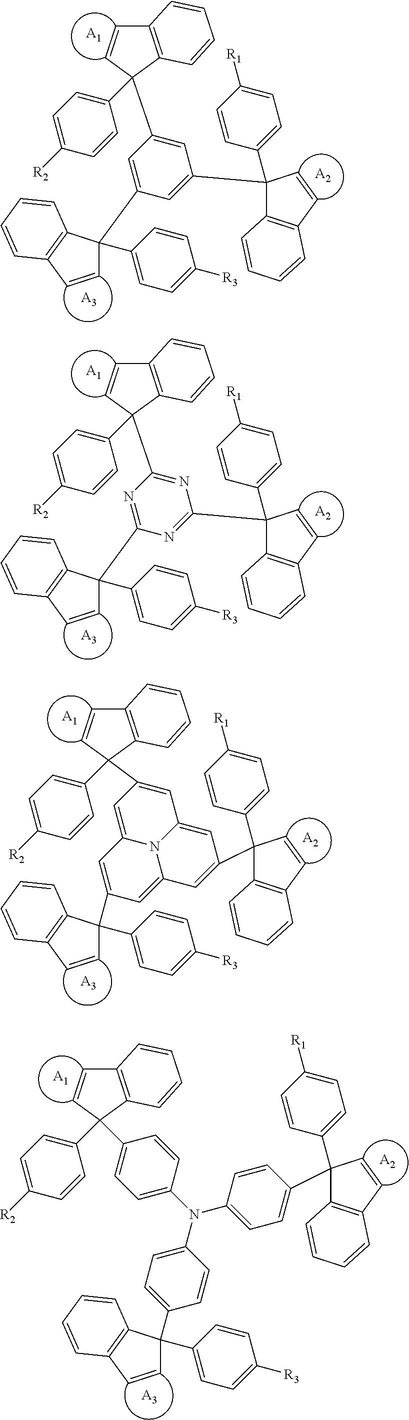 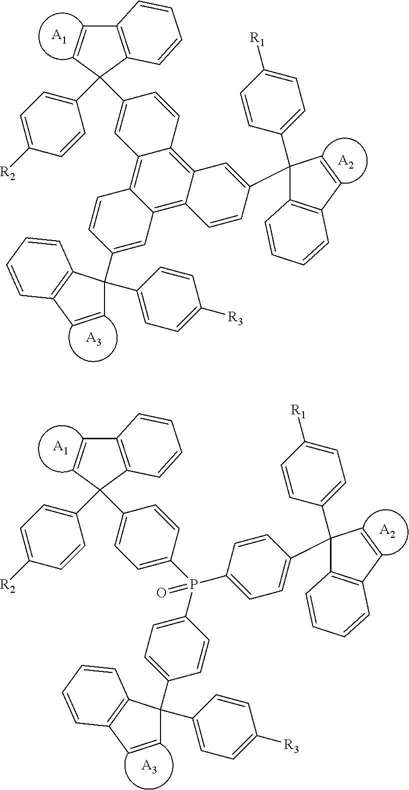
wherein A₁, A₂, A₃, R₁, R₂ and R₃ are defined as described above.

11. The terbenzocyclopentadiene compound according to claim 1, which is one selected from compounds having the following structural formula:

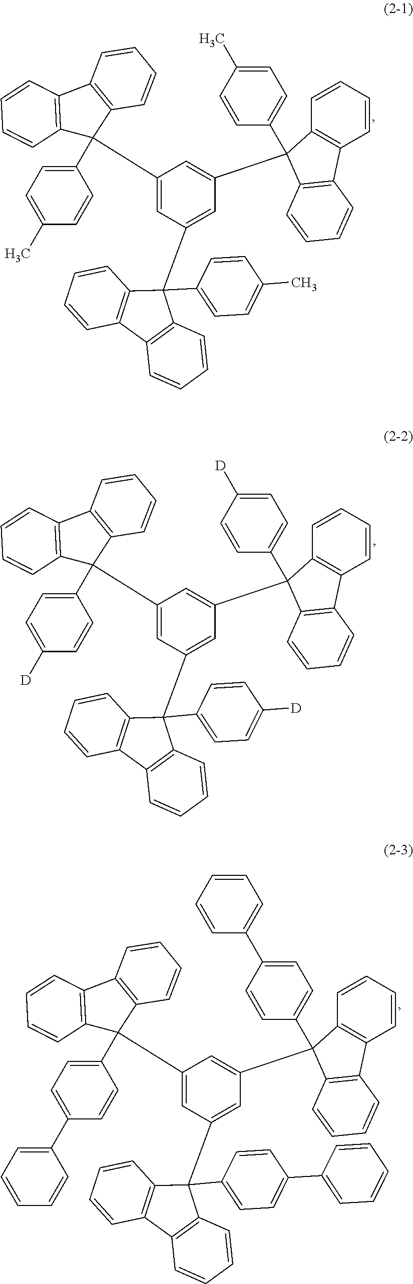

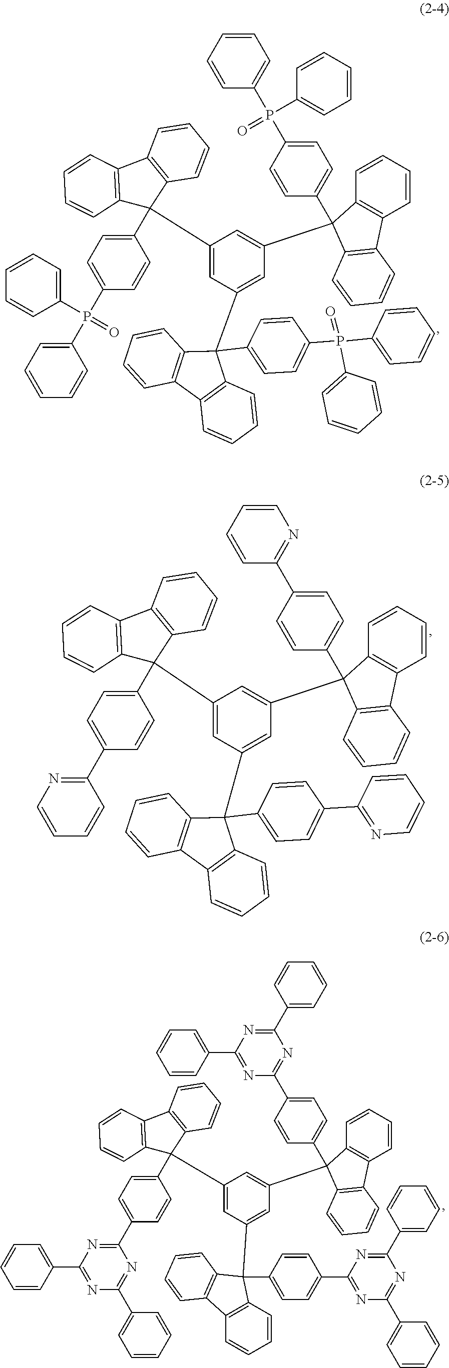

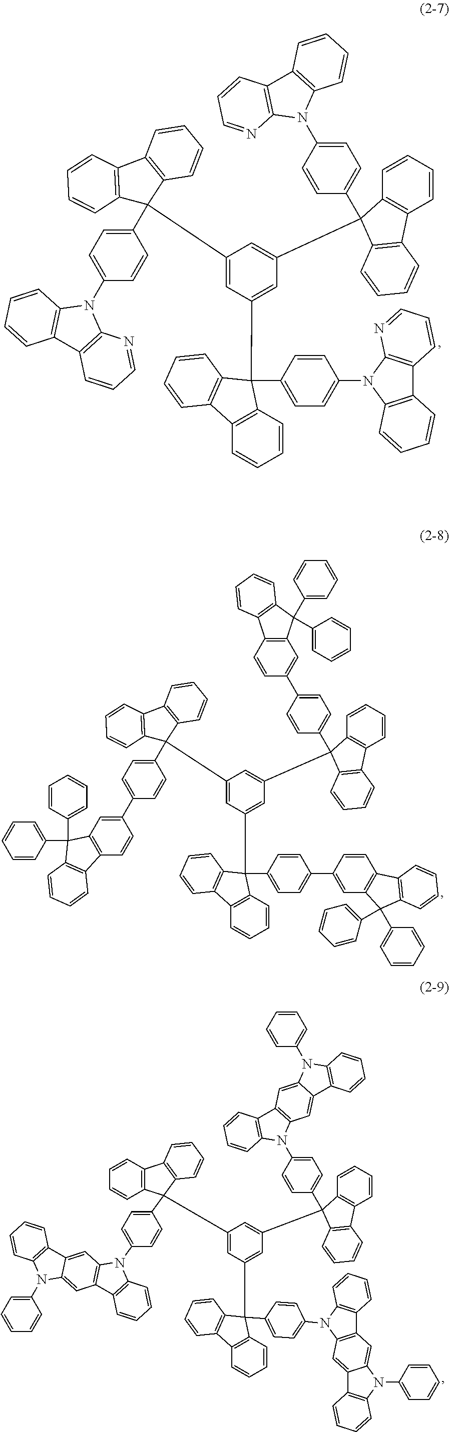

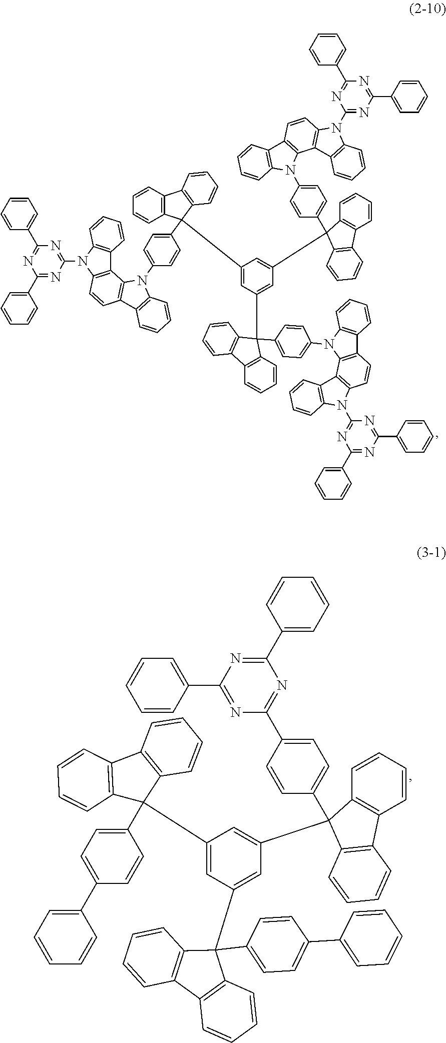

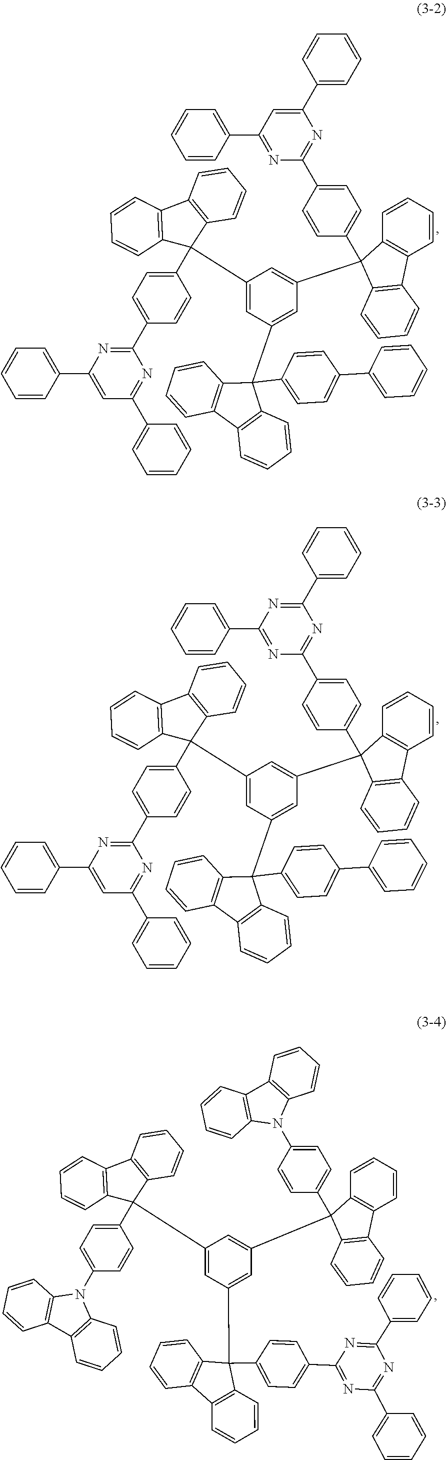

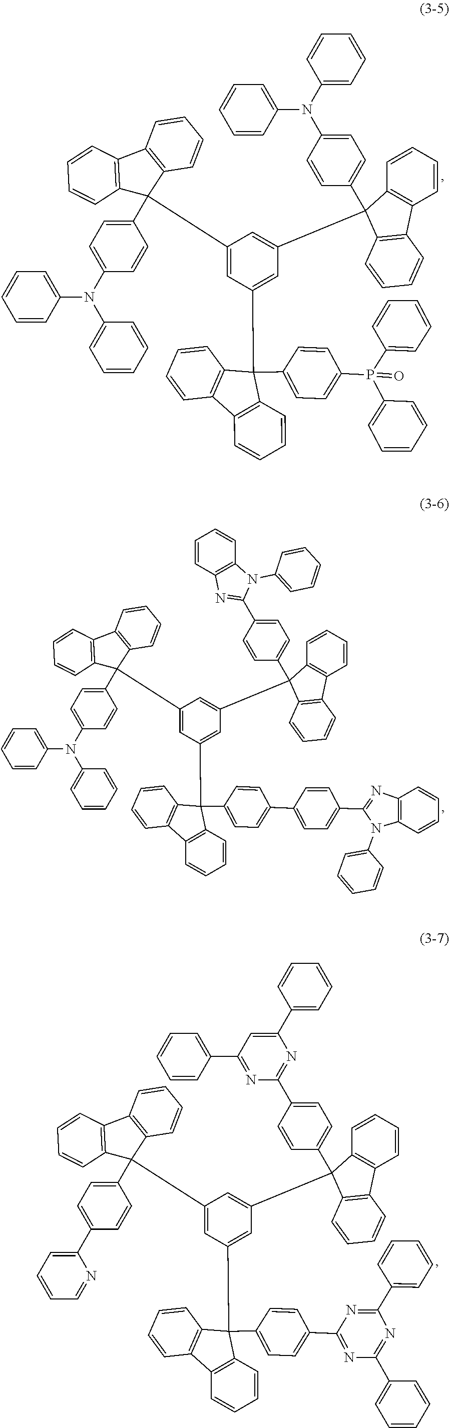

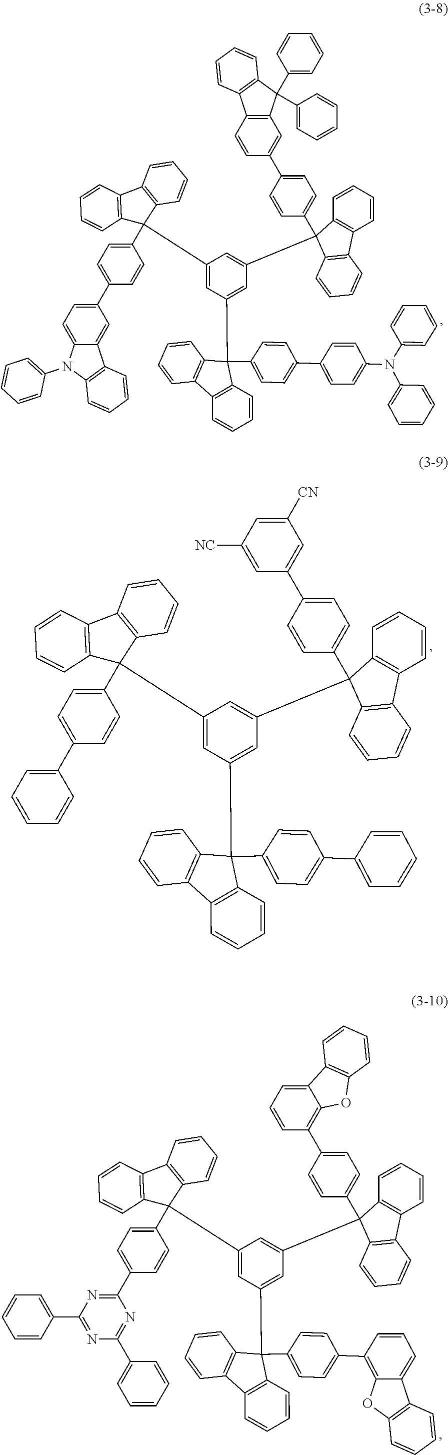

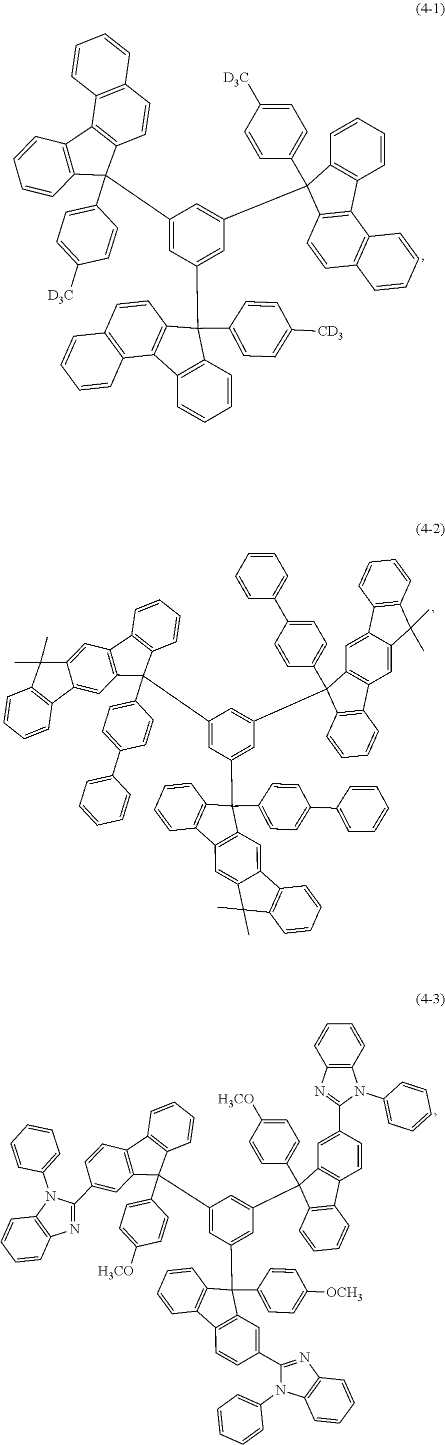

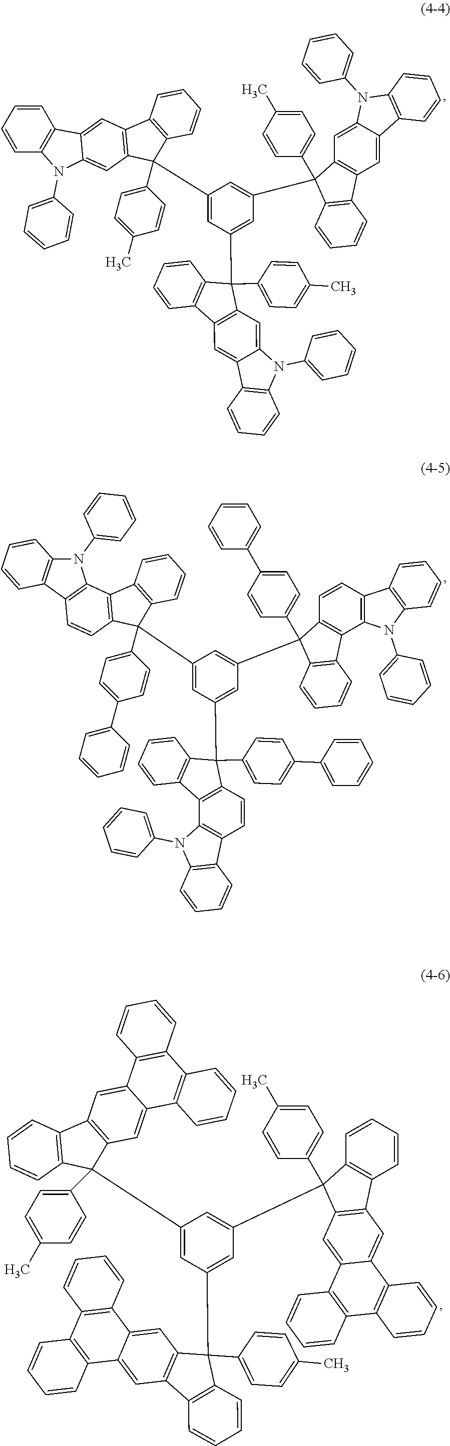

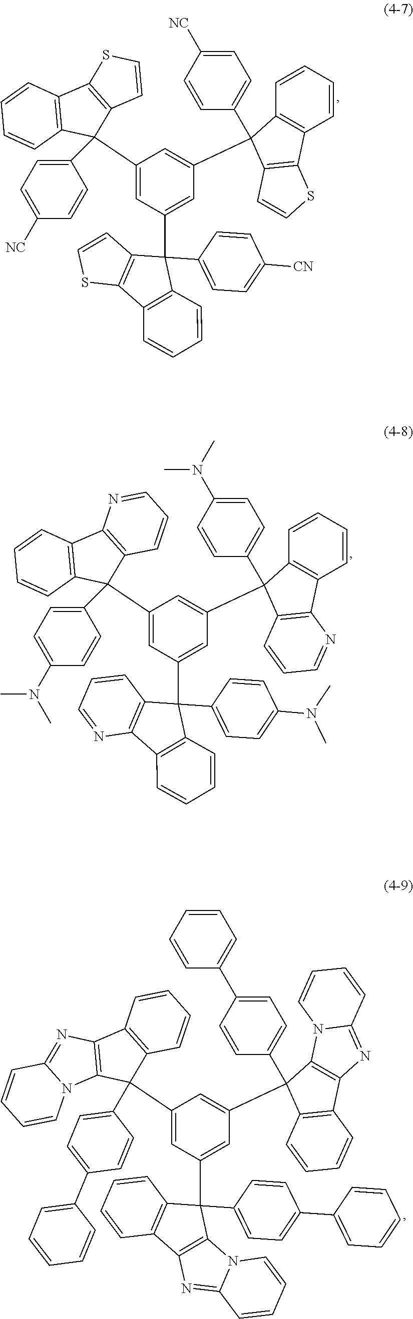 [SMILES: N#Cc1ccc(C2(c3cc(C4(c5ccc(C#N)cc5)c5ccccc5-c5sccc54)cc(C4(c5ccc(C#N)cc5)c5ccccc5-c5sccc54)c3)c3ccccc3-c3sccc32)cc1 ; CN(C)c1ccc(C2(c3cc(C4(c5ccc(N(C)C)cc5)c5ccccc5-c5ncccc54)cc(C4(c5ccc(N(C)C)cc5)c5ccccc5-c5ncccc54)c3)c3ccccc3-c3ncccc32)cc1 ; c1ccc(-c2ccc(C3(c4cc(C5(c6ccc(-c7ccccc7)cc6)c6ccccc6-c6nc7ccccn7c65)cc(C5(c6ccc(-c7ccccc7)cc6)c6ccccc6-c6nc7ccccn7c65)c4)c4ccccc4-c4nc5ccccn5c43)cc2)cc1]

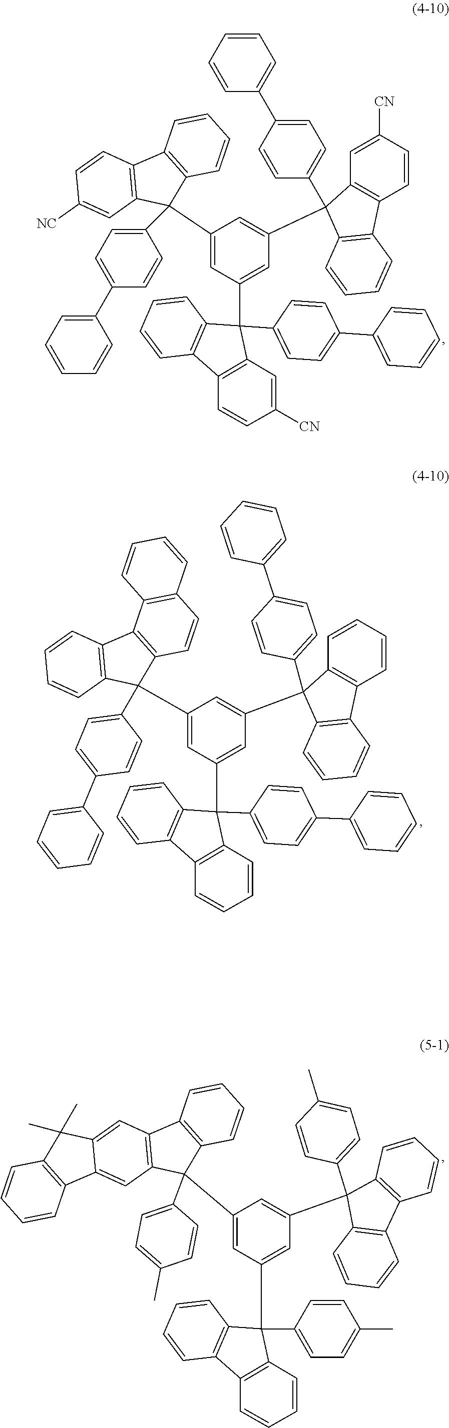

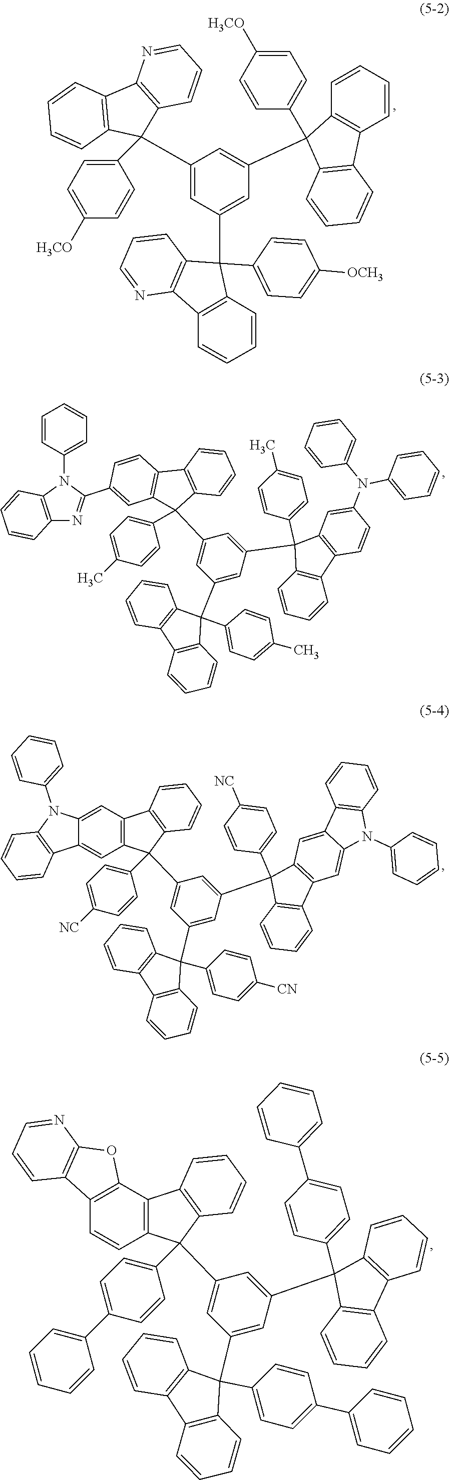

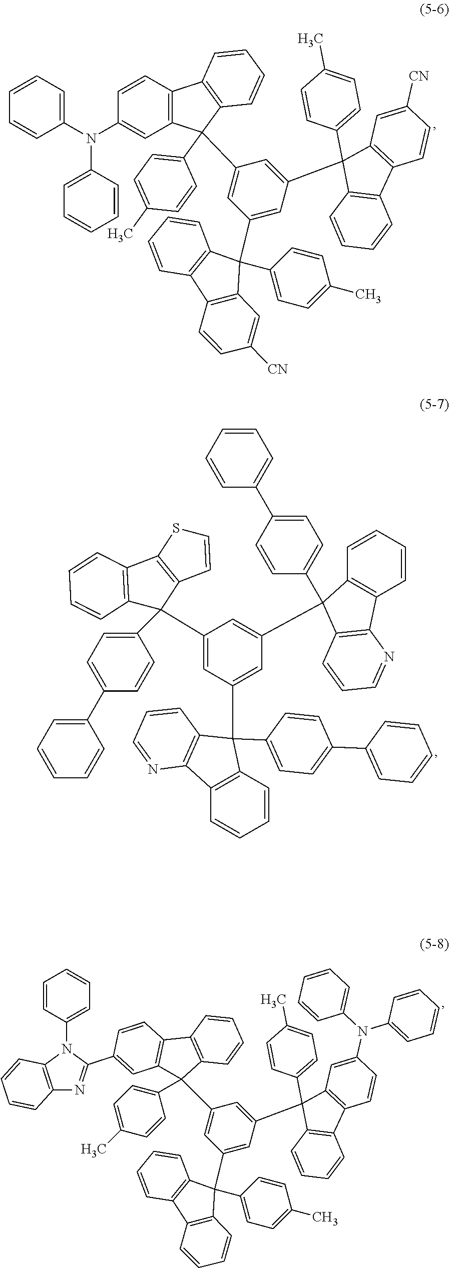 [SMILES: Cc1ccc(C2(c3cc(C4(c5ccc(C)cc5)c5ccccc5-c5ccc(C#N)cc54)cc(C4(c5ccc(C)cc5)c5ccccc5-c5ccc(N(c6ccccc6)c6ccccc6)cc54)c3)c3ccccc3-c3ccc(C#N)cc32)cc1 ; c1ccc(-c2ccc(C3(c4cc(C5(c6ccc(-c7ccccc7)cc6)c6ccccc6-c6ncccc65)cc(C5(c6ccc(-c7ccccc7)cc6)c6ccccc6-c6sccc65)c4)c4ccccc4-c4ncccc43)cc2)cc1 ; Cc1ccc(C2(c3cc(C4(c5ccc(C)cc5)c5ccccc5-c5ccc(-c6nc7ccccc7n6-c6ccccc6)cc54)cc(C4(c5ccc(C)cc5)c5ccccc5-c5ccc(N(c6ccccc6)c6ccccc6)cc54)c3)c3ccccc3-c3ccccc32)cc1]

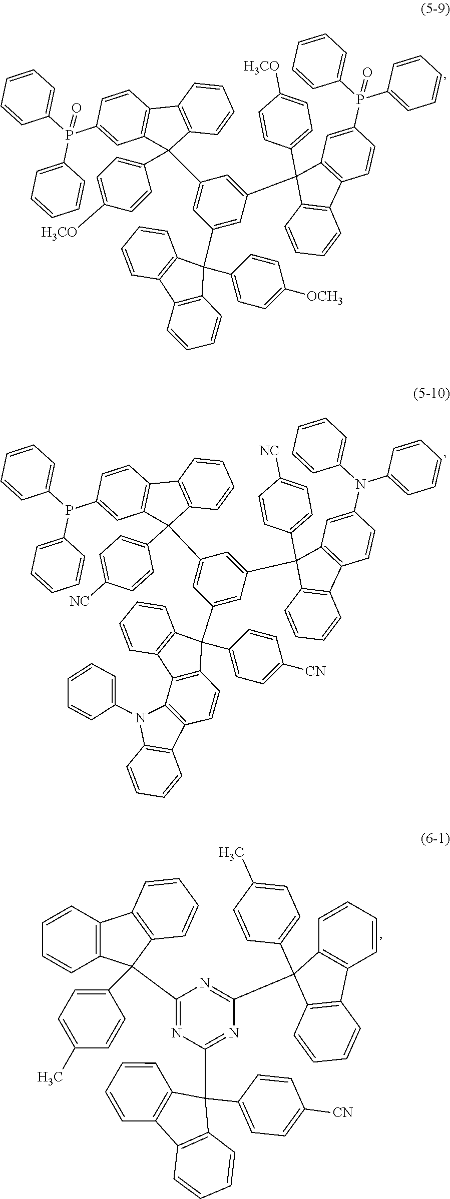 [SMILES: COc1ccc(C2(c3cc(C4(c5ccc(OC)cc5)c5ccccc5-c5ccc(P(=O)(c6ccccc6)c6ccccc6)cc54)cc(C4(c5ccc(OC)cc5)c5ccccc5-c5ccc(P(=O)(c6ccccc6)c6ccccc6)cc54)c3)c3ccccc3-c3ccccc32)cc1 ; N#Cc1ccc(C2(c3cc(C4(c5ccc(C#N)cc5)c5ccccc5-c5ccc(P(c6ccccc6)c6ccccc6)cc54)cc(C4(c5ccc(C#N)cc5)c5ccccc5-c5c4ccc4c6ccccc6n(-c6ccccc6)c54)c3)c3ccccc3-c3ccc(N(c4ccccc4)c4ccccc4)cc32)cc1 ; Cc1ccc(C2(c3nc(C4(c5ccc(C)cc5)c5ccccc5-c5ccccc54)nc(C4(c5ccc(C#N)cc5)c5ccccc5-c5ccccc54)n3)c3ccccc3-c3ccccc32)cc1]

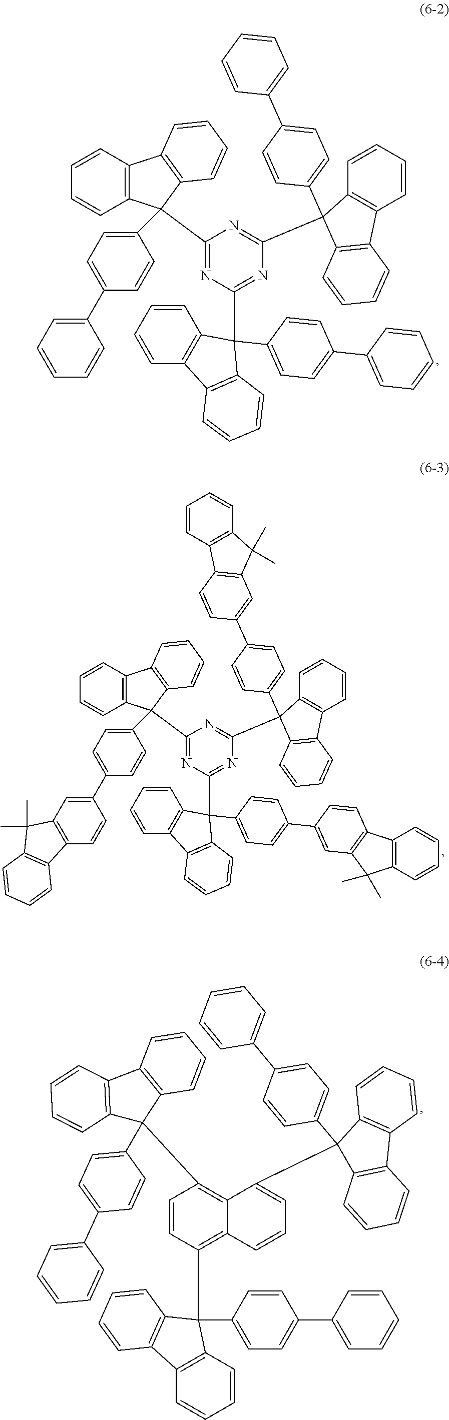

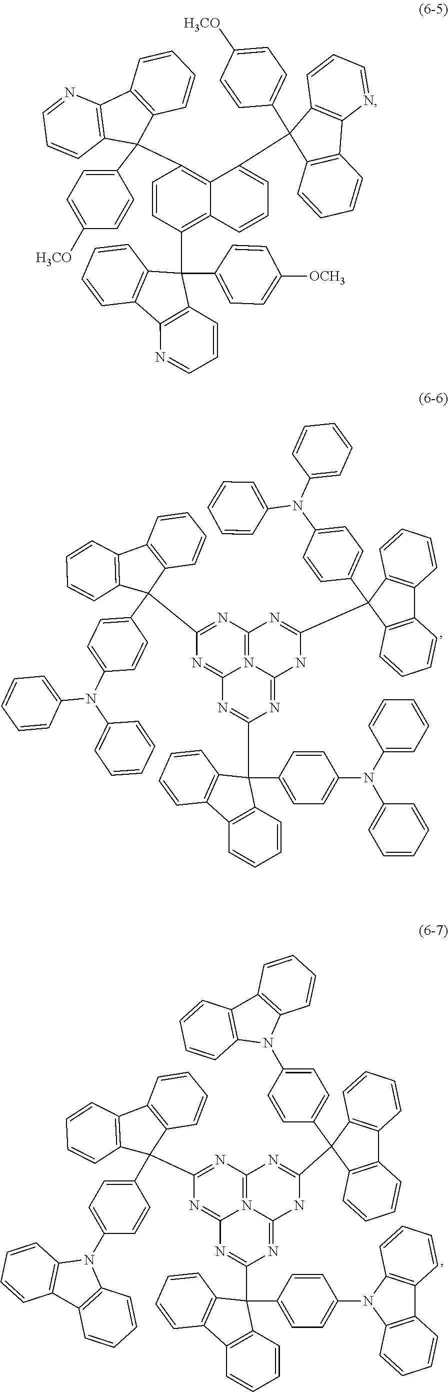

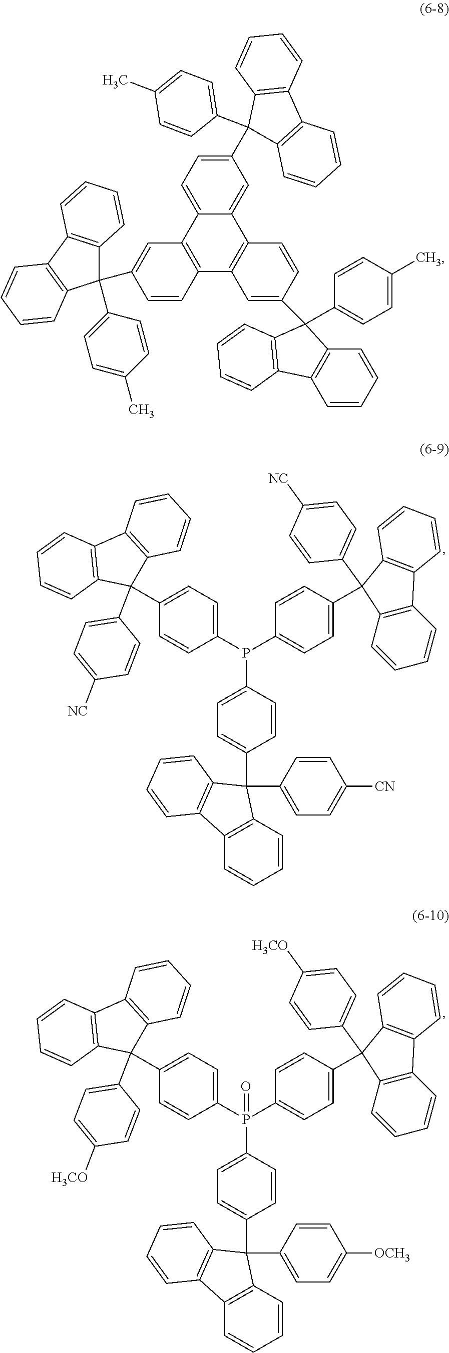

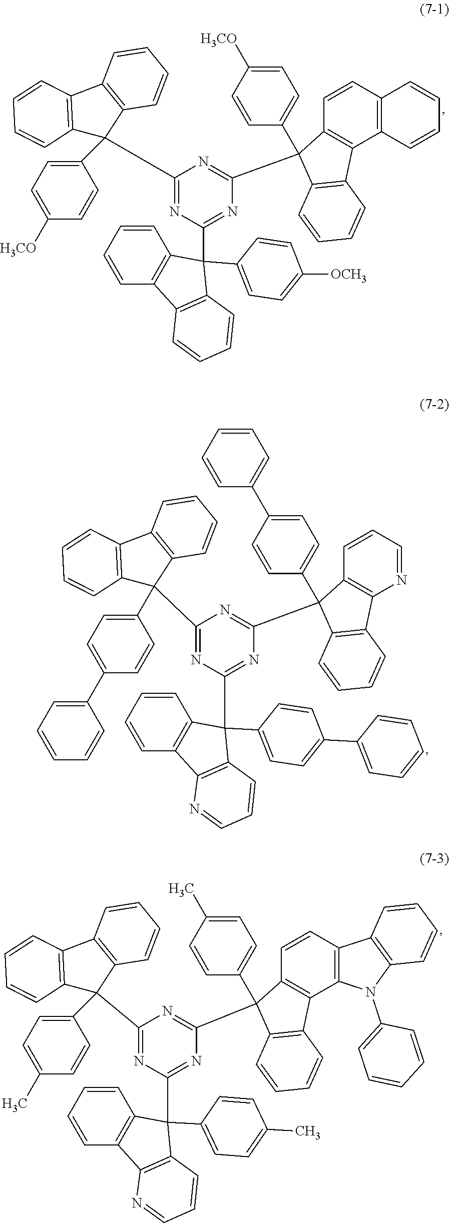 [SMILES: COc1ccc(C2(c3nc(C4(c5ccc(OC)cc5)c5ccccc5-c5ccccc54)nc(C4(c5ccc(OC)cc5)c5ccccc5-c5c4ccc4ccccc54)n3)c3ccccc3-c3ccccc32)cc1 ; c1ccc(-c2ccc(C3(c4nc(C5(c6ccc(-c7ccccc7)cc6)c6ccccc6-c6ncccc65)nc(C5(c6ccc(-c7ccccc7)cc6)c6ccccc6-c6ncccc65)n4)c4ccccc4-c4ccccc43)cc2)cc1 ; Cc1ccc(C2(c3nc(C4(c5ccc(C)cc5)c5ccccc5-c5ncccc54)nc(C4(c5ccc(C)cc5)c5ccccc5-c5c4ccc4c6ccccc6n(-c6ccccc6)c54)n3)c3ccccc3-c3ccccc32)cc1]

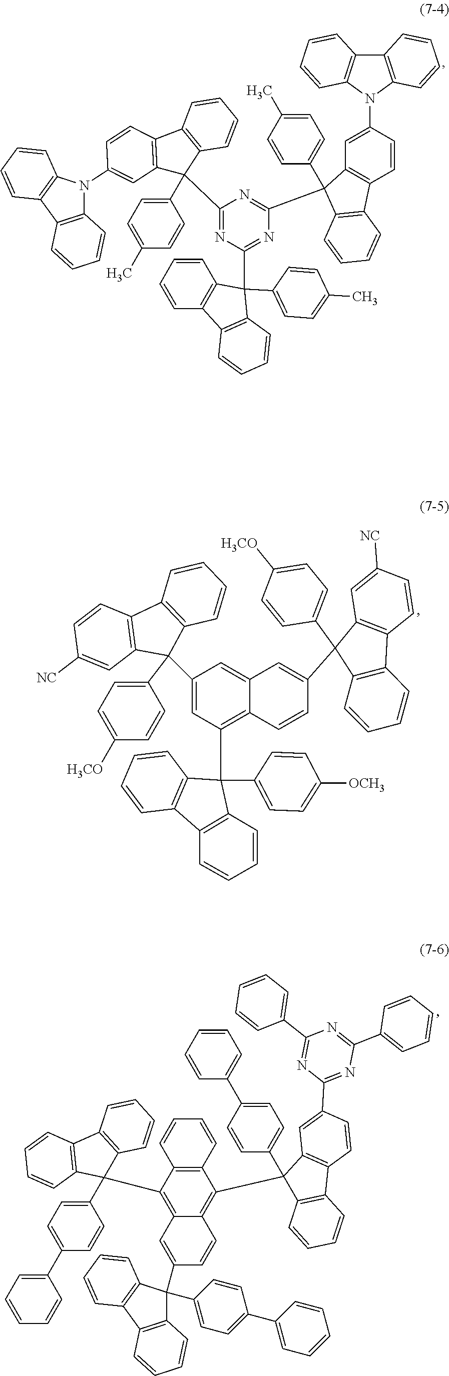 [SMILES: Cc1ccc(C2(c3nc(C4(c5ccc(C)cc5)c5ccccc5-c5ccc(-n6c7ccccc7c7ccccc76)cc54)nc(C4(c5ccc(C)cc5)c5ccccc5-c5ccc(-n6c7ccccc7c7ccccc76)cc54)n3)c3ccccc3-c3ccccc32)cc1 ; COc1ccc(C2(c3ccc4c(C5(c6ccc(OC)cc6)c6ccccc6-c6ccccc65)cc(C5(c6ccc(OC)cc6)c6ccccc6-c6ccc(C#N)cc65)cc4c3)c3ccccc3-c3ccc(C#N)cc32)cc1 ; c1ccc(-c2ccc(C3(c4ccc5c(C6(c7ccc(-c8ccccc8)cc7)c7ccccc7-c7ccc(-c8nc(-c9ccccc9)nc(-c9ccccc9)n8)cc76)c6ccccc6c(C6(c7ccc(-c8ccccc8)cc7)c7ccccc7-c7ccccc76)c5c4)c4ccccc4-c4ccccc43)cc2)cc1]

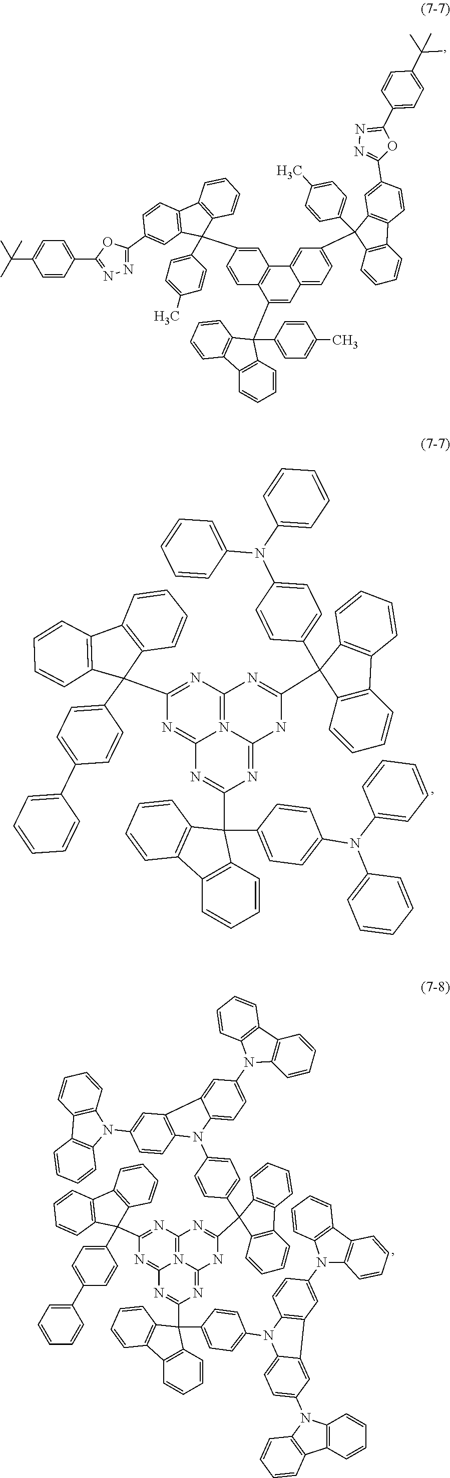

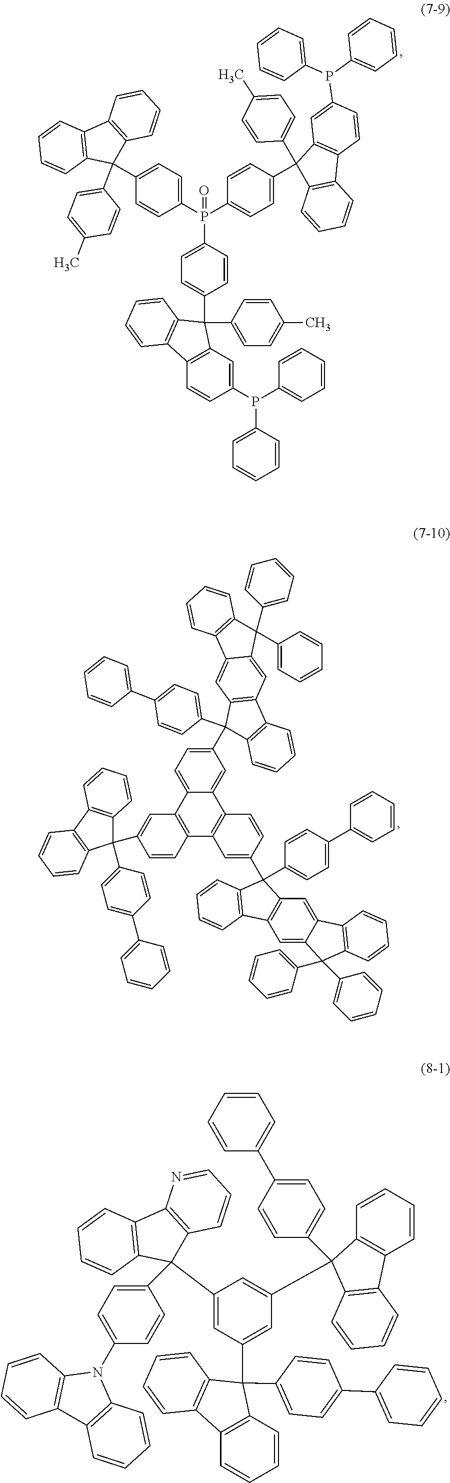

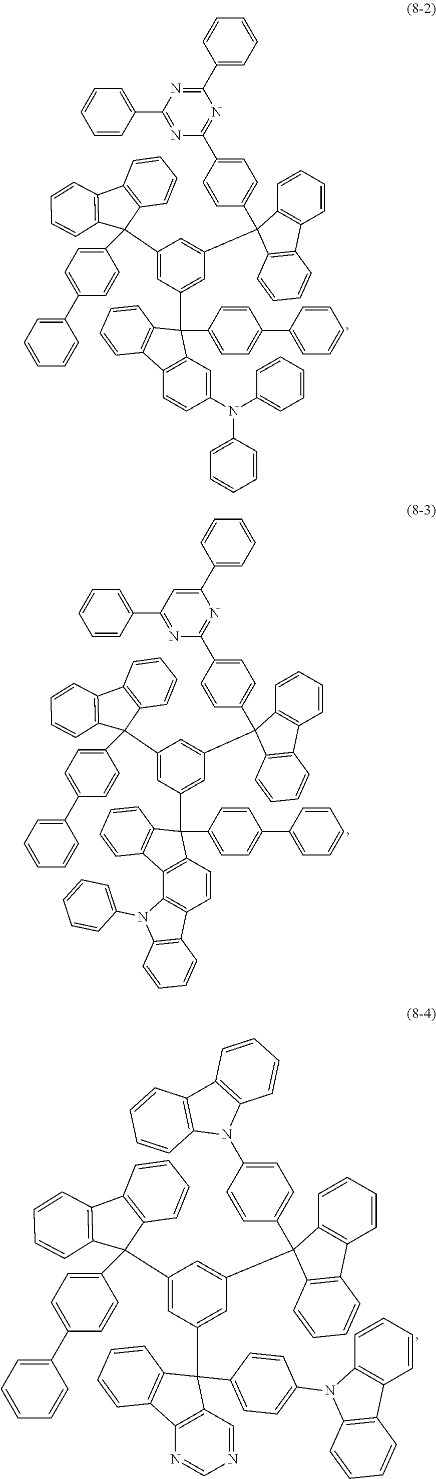

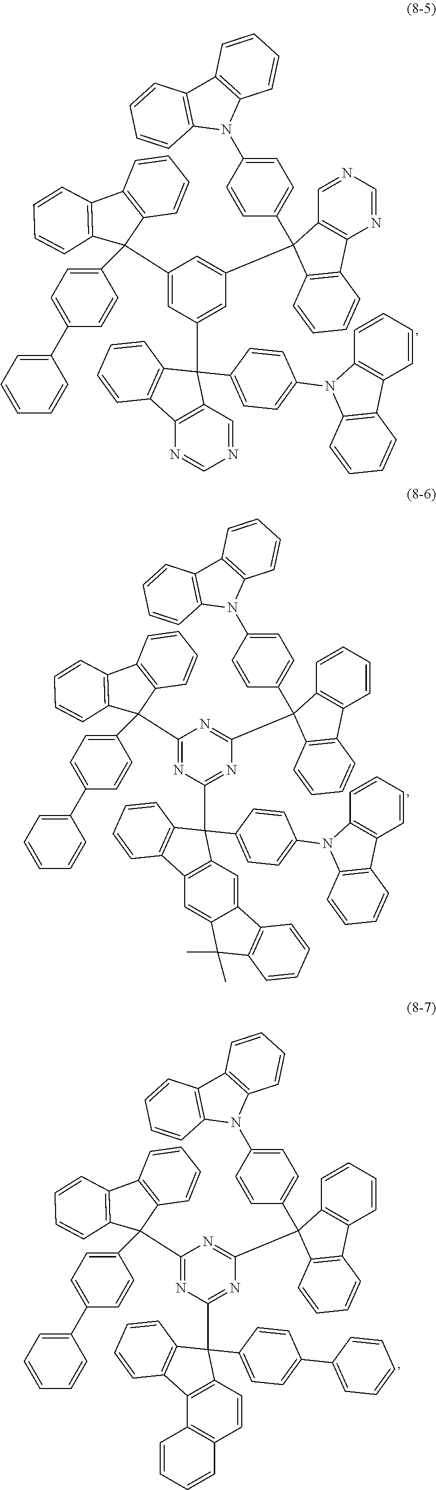 [SMILES: c1ccc(-c2ccc(C3(c4cc(C5(c6ccc(-n7c8ccccc8c8ccccc87)cc6)c6ccccc6-c6ncncc65)cc(C5(c6ccc(-n7c8ccccc8c8ccccc87)cc6)c6ccccc6-c6ncncc65)c4)c4ccccc4-c4ccccc43)cc2)cc1 ; CC1(C)c2ccccc2-c2cc3c(cc21)-c1ccccc1C3(c1ccc(-n2c3ccccc3c3ccccc32)cc1)c1nc(C2(c3ccc(-c4ccccc4)cc3)c3ccccc3-c3ccccc32)nc(C2(c3ccc(-n4c5ccccc5c5ccccc54)cc3)c3ccccc3-c3ccccc32)n1 ; c1ccc(-c2ccc(C3(c4nc(C5(c6ccc(-n7c8ccccc8c8ccccc87)cc6)c6ccccc6-c6ccccc65)nc(C5(c6ccc(-c7ccccc7)cc6)c6ccccc6-c6c5ccc5ccccc65)n4)c4ccccc4-c4ccccc43)cc2)cc1]

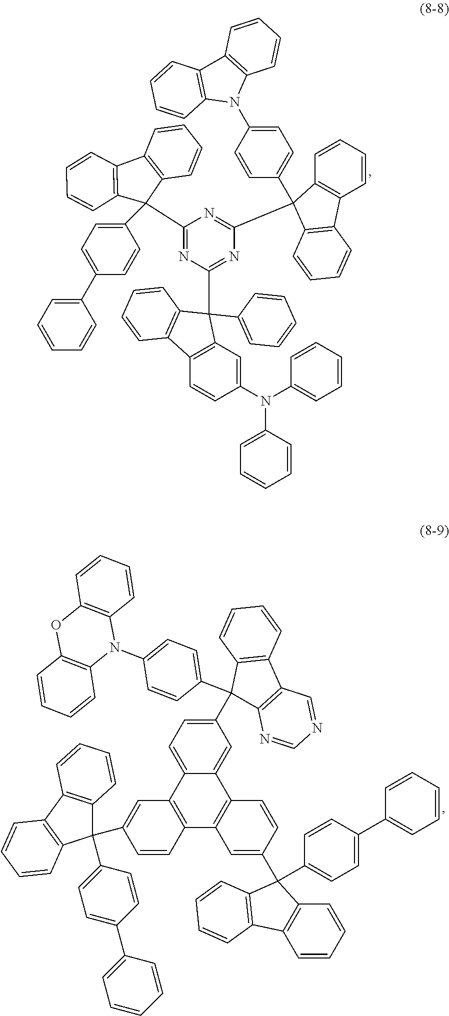 [SMILES: c1ccc(-c2ccc(C3(c4nc(C5(c6ccc(-n7c8ccccc8c8ccccc87)cc6)c6ccccc6-c6ccccc65)nc(C5(c6ccccc6)c6ccccc6-c6ccc(N(c7ccccc7)c7ccccc7)cc65)n4)c4ccccc4-c4ccccc43)cc2)cc1 ; c1ccc(-c2ccc(C3(c4ccc5c(c4)c4ccc(C6(c7ccc(-c8ccccc8)cc7)c7ccccc7-c7ccccc76)cc4c4ccc(C6(c7ccc(N8c9ccccc9Oc9ccccc98)cc7)c7ccccc7-c7cncnc76)cc54)c4ccccc4-c4ccccc43)cc2)cc1]

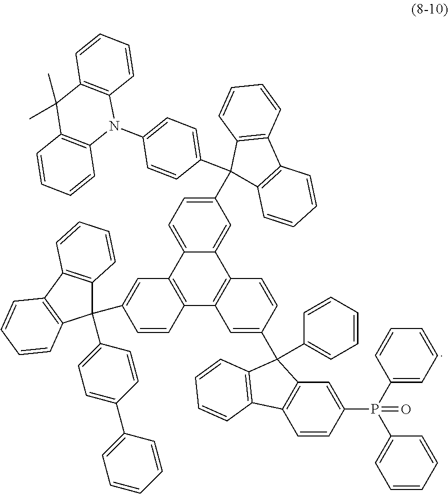 [SMILES: CC1(C)c2ccccc2N(c2ccc(C3(c4ccc5c(c4)c4ccc(C6(c7ccccc7)c7ccccc7-c7ccc(P(=O)(c8ccccc8)c8ccccc8)cc76)cc4c4ccc(C6(c7ccc(-c8ccccc8)cc7)c7ccccc7-c7ccccc76)cc54)c4ccccc4-c4ccccc43)cc2)c2ccccc21]

12. A polymer, wherein the polymer comprises at least one repeating unit represented by the general formula (1) comprised in the terbenzocyclopentadiene compound according to any one of claims 1 to 10.

13. A mixture, wherein the mixture comprises the terbenzocyclopentadiene compound according to any one of claims 1 to 11 or the polymer according to claim 12, and another organic functional material.

14. The mixture according to claim 13, wherein the another organic functional material is at least one selected from a hole injection material, a hole transport material, an electron injection material, an electron transport material, a hole blocking material, an electron blocking material, an organic host material, a singlet emitter, a multiplet emitter, a light-emitting organic metal complex, and an organic dye.

15. A formulation, wherein the formulation comprises the terbenzocyclopentadiene compound according to any one of claims 1 to 11 or the polymer according to claim 12, and an organic solvent.

16. An organic electronic device, wherein the organic electronic device comprises a terbenzocyclopentadiene compound according to any one of claims 1 to 11 or the polymer according to claim 12.

17. The organic electronic device according to claim 16, wherein the organic electronic device is one selected from an organic light-emitting diode, an organic photovoltaic, an organic light emitting cell, an organic field effect transistor, an organic light emitting field effector, an organic sensor, and an organic plasmon emitting diode.

18. The organic electronic device according to claim 16, wherein the organic electronic device is an electroluminescence device comprising a light-emitting layer; the light-emitting layer comprises the terbenzocyclopentadiene compound according to any one of claims 1 to 11 or the polymer according to claim 12.

19. The organic electronic device according to claim 16, wherein the organic electronic device is an electroluminescence device comprising a hole transport layer or an electron transport layer, or both;
wherein the hole transport layer comprising the terbenzocyclopentadiene compound according to any one of claims 1 to 11 or the polymer according to claim 12;
the electron transport layer comprising the terbenzocyclopentadiene compound according to any one of claims 1 to 11 or the polymer according to claim 12.
